(19) United States
(12) Patent Application Publication
(10) Pub. No.: US 2026/0306585 A1
(43) Pub. Date: Oct. 8, 2026

(54) METHODS OF DECREASING PATHOLOGICAL NEUROINFLAMMATION

(71) Applicant: Washington University, St. Louis, MO (US)

(72) Inventors: David Holtzman; Xiaoying Chen, St. Louis, MO (US); Jason Ulrich, St. Louis, MO (US)

(21) Appl. No.: 19/146,817

(22) PCT Filed: Jan. 9, 2024
(86) PCT No.: PCT/US2024/010921

§ 371 Date: Jul. 9, 2025

Related U.S. Application Data
(60) Provisional application No. 63/438,128, filed on Jan. 10, 2023.
(60) Provisional application No. 63/443,316, filed on Feb. 3, 2023.

(51) Int. Cl. A61K 38/17; A61K 31/137; A61K 31/444; A61K 31/445; A61K 39/00; A61P 25/28; C07K 16/28

(52) U.S. Cl. CPC A61K 38/1774; A61K 31/137; A61K 31/444; A61K 31/445; A61P 25/28; C07K 16/28; C07K 16/2818; C07K 16/2833; C07K 16/2842; C07K 16/2845; A61K 2039/505; C07K 2317/76

(57) ABSTRACT

The present disclosure relates to decreasing pathological neuroinflammation, particularly neuroinflammation associated with tauopathy and neurodegeneration. These pathologies can include Alzheimer's disease, certain types of dementia, Pick's disease, and ganglioglioma. Certain effects of decreasing pathological neuroinflammation may include a reduction in brain atrophy, a reduction neurodegeneration, a reduction in the interaction microglia and the brain parenchyma.

### FIELD

The present disclosure relates to decreasing pathological neuroinflammation, particularly neuroinflammation associated with tau-mediated neurodegeneration.

### BACKGROUND

Extracellular amyloid-β (AB) deposition as neuritic plaques and intracellular accumulation of hyperphosphorylated, aggregated tau as neurofibrillary tangles (NFT) are two of the characteristic hallmarks in Alzheimer's disease (AD). The regional progression of brain atrophy in AD highly correlates with tau accumulation but not amyloid deposition and the mechanisms of tau-mediated neurodegeneration remain elusive. Innate immune responses represent a common pathway for the initiation and progression of some neurodegenerative diseases. To date, little is known about the extent or role of the adaptive immune response in the presence of Aβ or tau pathology.

### SUMMARY

In one aspect, provided herein is a method of decreasing tau-mediated neurodegeneration in a subject in need thereof, the method of which may comprise administering an agent to the subject that inhibits T-cell activation, decreases T-cell numbers, inhibits microglia activation, or decreases microglia number.

In another aspect, provided herein is a method of decreasing central nervous system (CNS) p-tau in a subject in need thereof, the method of which may comprise administering an agent to the subject that inhibits T-cell activation, decreases T-cell numbers, inhibits microglia activation, or decreases microglia number.

In another aspect, provided herein is a method of reducing brain atrophy in a tau positive subject, the method of which may comprise administering an agent to the subject that inhibits T-cell activation, decreases T-cell numbers, inhibits microglia activation, or decreases microglia number.

In another aspect, provided herein is a method of reducing Nfl release from axons in the brain of a subject in need thereof, the method of which may comprise administering an agent to the subject that inhibits T-cell activation, decreases T-cell numbers, inhibits microglia activation, or decreases microglia number.

In another aspect, provided herein is a method of reducing reactive microglia in the brain parenchyma of a subject in need thereof, the method of which may comprise administering to the subject an agent that inhibits T-cell activation or decreases T-cell numbers in the brain parenchyma.

In another aspect, provided herein is a method of decreasing brain atrophy associated with tau pathology in a subject in need thereof, the method of which may comprise administering an agent to the subject that disrupts the interaction between activated microglia and parenchyma infiltrated T cells in the brain of the subject.

An agent as described herein and above may bind to CD80 or CD86. The agent may inhibit or decrease T-cell activation. The agent may be abatacept. The agent may be an anti-VLA-4 antibody or an antigen-binding fragment thereof, optionally selected from natalizumab. The agent may be a VLA-4 inhibitor, optionally selected from GW559090. The agent may inhibit the activity of, or decrease the amount of IFNgamma in the subject, optionally the agent may be an antibody or an antigen-binding fragment thereof. The agent may be a small molecule. The agent may be a CSF1R/c-kit/FLT3 inhibitor, optionally selected from PLX3397. The agent may reduce CD4+ cells, reduce CD8+ cells, or reduce both CD4+ and CD8+ cells. The agent may be an antibody that specifically binds P2ryl2, MHCII or CD11c, or an antigen-binding fragment thereof. The agent may be an immune checkpoint inhibitor, optionally selected from an anti-PD-1 (αPD-1) antibody or antigen-binding fragment thereof. The agent may be fingolimod. The agent may be a JAK inhibitor.

Microglia as of a method as described herein and above may be disease associated microglia, or interferon-activated microglia.

A subject of a method as described herein and above may have or is suspected of having Alzheimer's disease and/or a tau pathology, or a tau pathology but no clinical signs of Alzheimer's disease.

### BRIEF DESCRIPTION OF THE FIGURES

The patent or patent application file contains at least one drawing executed in color. Copies of this patent or patent application publication with color drawing(s) will be provided by the Office upon request and payment of the necessary fee.

Those of skill in the art will understand that the figures, described below, are for illustrative purposes only. The figures are not intended to limit the scope of the present teachings in any way.

FIG. 1A displays representative images of 6-month E4 and TE4; 9.5-month E4, TE4, A/PE4 and 5×E4 mouse brain sections stained with Sudan black. Scale bar=1 mm. FIG. 1B-1D depict volumes of hippocampus, entorhinal/piriform cortex and posterior lateral ventricle in 6-month E4 and TE4; 9.5-month E4, TE4, A/PE4, 5×E4 and WT mice. (E4-6m: n=3, TE4-6m: n=7, E4-9.5m: n=15, TE4-9.5m n=13, A/PE4-9.5m: n=7, 5×E4-9.5m: n=6 and WT-9.5m: n=6). ***p<0.0001 for 9.5m, TE4 vs. A/PE4; TE4 vs. 5×E4; TE4 vs. E4 and TE4 vs. WT. Ent: Entorhinal. Piri: Piriform. One-way ANOVA with Tukey's post hoc test. FIG. 1E illustrates FACS sorting of CD45^{Total} and/or CD45^{high} cells from brain parenchyma and meninges from E4, A/PE4 and TE4 mice for single cell immune RNA-seq. (FIG. 1F) CD45^{Total} immune cells from brain parenchyma assigned into 12 cell types as visualized by UMAP plot. FIG. 1G depicts bar plot showing the proportions of the 12 cell types of immune cells in the brain parenchyma. FIG. 1H illustrates CD45^{Total} immune cells from meninges assigned into 12 cell types as visualized by UMAP plot. FIG. 1I depicts Bar plot showing the proportions of the 12 cell types of immune cells in the meninges.

FIG. 2A shows representative image of 9.5-month A/PE4 mouse brain sections stained with an anti-AB antibody and X-34. Scale bar=500 μm. FIG. 2B displays representative images of 6 and 9.5-month TE4 mouse brain sections stained with AT8 antibody. Scale bar=500 μm. FIG. 2C depicts quantification of brain regional volumes of 9.5-month mice normalized to 6-month in b. TE4-6 months: n=7, TE4-9.5 months: n=13. ***p<0.0001 for hippocampus (Hip) vs. cortex dorsal to the hippocampus (Ctx); amygdala (Amyg) vs. Ctx and entorhinal/piriform cortex (Ent) vs. Ctx. One-way ANOVA with Tukey's post hoc test. FIG. 2D illustrates quantification of the area covered by AT8 of 9.5-month TE4 mouse brain sections in FIG. 2B. TE4-6 months: n=7, TE4-9.5 months: n=13. ***p<0.0001 for Hip vs. Ctx; Amyg vs. Ctx and Ent vs. Ctx. One-way ANOVA with Tukey's post hoc test. FIG. 2E displays representative images of 6-month TE4, 9.5-month E4, and 9.5-month TE4 mouse brain sections stained with NeuN. Scale bar=50 μm. FIG. 2F illustrates thickness of granule cell layer of the dentate gyrus in 9.5-month E3, TE3, E4, TE4 mice. (E3: n=5, TE3: n=6, E4: n=15 and TE4: n=13). *p=0.0130 for TE3 vs. TE4. Two-way ANOVA with Tukey's post hoc test. FIG. 2G displays correlation between DG neuronal layer thickness and hippocampal volume. n=39 biologically independent animals from FIG. 2F. Person correlation analysis. p<0.0001, R2=0.8335. FIGS. 2H-21 display representative images of 9.5-month E4 and TE4 mouse brain sections stained with MBP. Scale bar=500 μm in h. Scale bar=100 μm in i. FIGS. 2J-2L show volumes of hippocampus, entorhinal/piriform cortex and posterior lateral ventricle in 9.5-month E3, TE3, E4, TE4 mice. (E3: n=5, TE3: n=6, E4: n=15 and TE4=13). p=0.0505 for TE3 vs. TE4 in comparing the volume of the hippocampus, *p=0.0207 for TE3 vs. TE4 in comparing the volume of the posterior lateral ventricle. Two-way ANOVA with Tukey's post hoc test. FIGS. 2M-20 show volumes of hippocampus, entorhinal/piriform cortex and posterior lateral ventricle in 9.5-month TE3, TE4, A/PE4, 5×E4 male and female mice. (TE3-M: n=6, TE3-F: n=11, TE4-M: n=13, TE4-F: n=17, A/PE4-M: n=7, A/PE4-F: n=6, 5×E4-M: n=6, 5×E4-F: n=7). **p=0.0087 for TE4 male vs. female entorhinal/piriform cortex volume. Two-way ANOVA with Tukey's post hoc test.

FIG. 3A illustrates FACS sorting of CD45Total and/or CD45high cells from brain parenchyma and meninges from A/PE4 mice for single cell immune RNA-seq. FIG. 3B displays representative cell type specific makers in brain parenchyma (CD45Total) clusters. FIG. 3C depicts CD45high immune cells from parenchyma assigned into 12 cell types as visualized by UMAP plot. FIG. 3D shows the analysis of the CD4 and CD8 positive T cells present in the brain of E4 and TE4 mice by flow cytometry. ***p<0.0001, Unpaired two-tailed Student's t test. FIG. 3E depicts representative cell type specific makers in meninges (CD45Total).

FIG. 4A shows representative flow cytometry gating plot of splenic lymphocytes. FIG. 4B displays quantification of the proportion of indicated lymphocytes and their subsets among 9.5-month E4, A/PE4, and TE4 mice. FIG. 4C illustrates representative flow cytometry gating plot of splenic antigen presenting cells (APCs). FIG. 4D displays quantification of the proportion of indicated APCs among 9.5-month E4, A/PE4, and TE4 mice. FIG. 4E shows representative flow cytometry gating plot of splenic myeloid cells and minor lymphocytes including NK cells and NKT cells. FIG. 4F depicts quantification of the proportion of indicated cells among 9.5-month E4, A/PE4, and TE4 mice.

FIG. 5A displays Iba1 and CD3 staining in 6-month E4 and TE4, 9.5-month E4, TE4, A/PE4 and 5×E4 mice in dentate gyrus (DG). Scale bar=20 μm. FIG. 5B illustrates quantification of numbers of CD3+ T cells in DG per 0.3 mm². (E4-6m: n=3, TE4-6m: n=7, E4-9.5m: n=15, TE4-9.5m: n=13, A/PE4-9.5m: n=7 and 5×E4-9.5m: n=6). ***p<0.0001 for 9.5m, TE4 vs. A/PE4; TE4 vs. 5×E4 and TE4 vs. E4. One-way ANOVA with Tukey's post hoc test. FIG. 5C displays vessel and CD3 staining in 9.5-month TE4, 19-month A/PE4 and 5×E4 mice, Scale bar=20 μm. FIG. 5D illustrates quantification of the % area covered by Iba1 in DG. (E4-6m: n=3, TE4-6m: n=7, E4-9.5m: n=15, TE4-9.5m n=13, E3-9.5m: n=5 and TE3-9.5m: n=6). ***p<0.0001 for TE4-9.5m vs. E4-9.5m; and *p=0.0276 for TE3-9.5m vs. E3-9.5m. One-way ANOVA with Tukey's post hoc test. FIG. 5E depicts Correlation between the area covered by Iba1 and CD3+ T cells. n=49 biologically independent animals from d. Person correlation analysis. R2=0.902, p<0.0001. FIG. 5F depicts correlation between the number of CD3+ T cells with the granule cell layer thickness in DG. n=49 biologically independent animals from d. R2=0.7454. p<0.0001. FIG. 5G displays CD3, Aβ and AT8 staining in brain slices from AD patients (superior frontal gyrus) of low Braak stage I or II and high Braak stage VI. Scale bar=10 μm. FIG. 5H shows quantification of the area covered by AT8 in g. (Braak I or II: n=3; Braak VI: n=7). *p=0.0195. Unpaired two-tailed Student's t test. FIG. 5I illustrates quantification of numbers of CD3+ T cells per mm² in FIG. 5H. *p=0.0277. Unpaired two-tailed Student's t test. FIG. 5J shows quantification of the area covered by Aβ in h. (Braak I or II: n=3; Braak VI: n=7). p=0.1522. Unpaired two-tailed Student's t test.

FIG. 6A shows quantification of CD3 numbers per DG area with 0.3 mm² in 9.5 month E3, TE3, E4, TE4 mice. (E3: n=5, TE3: n=6, E4: n=15 and TE4: n=13). Two-way ANOVA with Tukey's post hoc test. p=0.1342 for TE3 vs. TE4. FIG. 6B displays representative images of 9.5-month old E4, TE4, A/PE4 and 5×E4 mouse brain sections stained with CD3, Tba1, Aβ and X-34. Scale bar=20 μm. FIG. 6C depicts TEM image demonstrating presence of a cell with T cell like features in brain parenchyma of 9.5 month of TE4 mouse. Scale bar=2 μm.

FIG. 7A shows Total T cells from brain parenchyma and meninges assigned into 15 categories as visualized by UMAP plot. FIG. 7B displays bar plot showing the percentages of T cells subgroups in E4, A/PE4 and TE4 mice. FIG. 7C depicts scatter plot illustrating differential TRAV and TRBV pairing expression in CD4⁺ T cells in TE4 vs. E4 (x axis) and A/PE4 vs. E4 (y axis) mice. FIG. 7D shows CD4⁺ T cells from brain parenchyma assigned into 6 cell types as visualized by UMAP plot. FIG. 7E displays representative TRAV-TRBV paring projection in CD4⁺ T cells in E4, A/PE4 and TE4 mice. FIG. 7F illustrates scatter plot illustrating differential TRAV and TRBV pairing expression in CD8⁺ T cells in TE4 vs. E4 (x axis) and A/PE4 vs. E4 (y axis) mice. FIG. 7G illustrates CD8⁺ T cells from brain parenchyma assigned into 10 cell types as visualized by UMAP plot. FIG. 7H depicts percentages of activated (cell types 3 and 6) and exhausted (cell type 1) CD8⁺ T cells in E4, A/PE4 and TE4 mice. FIG. 7I displays trajectory analysis showing naive CD8⁺ T cells demarcated into three paths, cell type 6, Itgax⁺Klre1⁺; cell type 3, ISg15⁺ and cell type 1, Tox⁺Pdcd1⁺. FIG. 7J illustrates representative TRAV-TRBV pairing projection in CD8⁺ T cells in E4, A/PE4 and TE4 mice.

FIG. 8A displays heatmap showing identified marker genes in each of the categorized cell types in Total T cells, CD4+ and CD8+ T cells. FIG. 8B shows total T cells from brain parenchyma and meninges with TCR assigned into 13 cell types as visualized by UMAP plot. FIG. 8C illustrates TRAV and TRBV enrichment in CD8+ and CD4+ T cells in TE4 and A/PE4 mice. FIG. 8D depicts representative TCR-TRBV projection in E4, A/PE4 and TE4 mice.

FIG. 9 shows quantification of cytokines, chemokines, growth factors and soluble receptors in brain lysates in 9.5 month old E4, TE4, and TEKO mice. (E4: n=10, TE4: n=10 and TEKO: n=10). One-way ANOVA with Tukey's post hoc test. With Q=0.1% identify outlier function, n=1 E4 and n=1 TEKO samples for IFN-g measurements were removed; n=1 E4 sample for IL-1ß measurements was removed.

FIG. 10A displays total microglia assigned into 3 subgroups, HOM microglia, DAM microglia and IFN activated microglia, as visualized by UMAP plot. FIG. 10B depicts heat map showing representative markers specifically expressed in 3 subgroups. FIG. 10C illustrates bar plot showing the percentages of the 3 subgroups of microglia in E4, A/PE4 and TE4 mice. FIG. 10D shows DEGs in TE4 vs. E4 and A/PE4 vs. E4 in microglia subgroups. FC, fold change. FIG. 10E shows Iba1 and CD3 staining in 9.5-month TE4-Ctrl and TE4-Plx mice. Scale bar=20 μm. FIG. 10F displays P2ryl2, MHCII and Iba1 staining in 9.5-month TE4-Ctrl and TE4-Plx mice. Scale bar=50 μm. FIG. 10G shows CD11c, CD8 and Iba1 staining in 9.5-month TE4-Ctrl and TE4-Plx mice. Scale bar=50 μm. FIGS. 10H-10K illustrate quantification of the covered areas of Iba1, P2ryl2, MHCII and CD11c in 9.5-month TE4-Ctrl and TE4-Plx mice. (TE4-Ctrl: n=5 and TE4-Plx: n=11). ***p<0.0001, ***p<0.0001, **p=0.0031, ***p<0.0001 for TE4-Plx vs. TE4-Ctrl in comparison of the areas covered by Iba1, P2ryl2, MHCII and CD11c, respectively. Unpaired two-tailed Student's t test. FIG. 10L shows representative images of 9.5-month E4-Plx, TE4-Ctrl and TE4-Plx mouse brain sections stained with Sudan black. Scale bar=1 mm. FIGS. 10M-10O display volumes of hippocampus, entorhinal/piriform cortex and posterior lateral ventricle in 9.5-month E4-Plx, TE4-Ctrl and TE4-Plx mice. (E4-Plx: n=12; TE4-Ctl: n=5 and TE4-Plx: n=11). *p=0.019 **p=0.009, for TE4-Ctrl vs. TE4-Plx in comparing of the volumes of hippocampus and posterior lateral ventricle, respectively. Unpaired two-tailed Student's t test. FIG. 10P shows quantification of the number of CD3+ T cells in DG per 0.3 mm² in 9.5-month E4-Plx, TE4-Ctrl and TE4-Plx mice. (E4-Plx: n=12; TE4-Ctl: n=5 and TE4-Plx: n=11). *p=0.0236 for TE4-Ctrl vs. TE4-Plx. Unpaired two-tailed Student's t test. FIG. 10Q illustrates quantification of the number of CD8+ T cells in DG per slice in 9.5-month TE4-Ctl and TE4-Plx mice. (TE4-Ctrl: n=5 and TE4-Plx: n=11). **p=0.002 for TE4-Ctrl vs. TE4-Plx. Unpaired two-tailed Student's t test. FIG. 10R depicts quantification of the area covered by AT8 in DG per slice in 9.5-month TE4-Ctrl and TE4-Plx mice. (TE4-Ctrl: n=5 and TE4-Plx: n=11). ***p=0.0008 for TE4-Ctrl vs. TE4-Plx. Unpaired two-tailed Student's t test.

FIG. 11A shows MHCII, CD206, Iba1 and GFAP staining in 9.5-month TE4 mice. Scale bar=100 μm. FIG. 11B displays Iba1, MHCII and Af3 staining in 9.5 month E4, A/PE4, TE4 and TEKO mice in Cortex dorsal to hippocampus, Hippocampus and Ent/Piri cortex. Scale bar=50 μm. FIG. 11C illustrates quantification of the area covered by MHCII in Ctx, Hip and Ent in 9.5-month TE4 and TEKO mice. (TE4: n=13 and TEKO: n=15). ***p<0.0001 for TE4-Hip vs. TEKO-Hip. One-way ANOVA with Tukey's post hoc test. FIG. 11D depicts Iba1, Cd11c and Af3 staining in 9.5-month E4, A/PE4, TE4 and TEKO mice in Prefrontal cortex, Hippocampus and Ent/Piri cortex. Scale bar=50 μm. FIG. 11E shows quantification of the area covered by CD11c in Ctx, Hip and Ent in 9.5-month TE4 and TEKO mice. (TE4: n=13 and TEKO: n=15). ***p<0.0001 for TE4-Hip vs. TEKO-Hip. One-way ANOVA with Tukey's post hoc test. FIG. 11F displays volume of hippocampus in 9.5-month TE4 and TEKO mice. (TE4: n=13 and TEKO: n=15). ***p<0.0001. Unpaired two-tailed Student's t test. FIG. 11G shows quantification of numbers of CD3+ T cells in DG with 0.3 mm². (TE4: n=13 and TEKO: n=15). ***p<0.0001. Unpaired two-tailed Student's t test.

FIG. 12A depicts ligand-receptor analysis in T cells and microglia. FIG. 12B shows IFN-γ expression in brain parenchyma (CD45Total) 12 cell types and T cells from brain parenchyma 15 clusters as visualized by UMAP plot. FIG. 12C illustrates the gating strategy for sorting naïve OT-1 CD8+ T cells, dendritic cells (DCs), and microglia. FIG. 12D displays representative flow cytometry plot to assess the proliferation of OT-1 T cells by cell tracer violet (CTV) dilution after 3 days of co-culture with APCs in the presence of OVA. FIG. 12E illustrates representative flow cytometry plot showing dose dependent OVA antigen presentation by DCs, microglia, or microglia in the presence of IFNγ assayed by OT-1 proliferation. FIG. 12F depicts graph of percent proliferating OT-1 T cells under the indicated conditions. Data are from one representative experiment. Two independent experiments were done showing similar results.

FIG. 13A displays representative images of 9.5-month TE3-IgG and TE3-αIFN-γ treated mouse brain sections stained with Sudan black. Scale bar=1 mm. FIG. 13B-13D show volumes of hippocampus, entorhinal/piriform cortex and posterior lateral ventricle in 9.5-month TE3-IgG and TE3-αIFNγ treated mice. (TE3-IgG: n=12 and TE3-αIFNγ: n=12). p=0.0479, 0.0398, 0.265 for TE3-IgG vs. TE3-αIFNγ in comparing the volumes of hippocampus, entorhinal/piriform cortex and posterior lateral ventricle, respectively. Unpaired two-tailed Student's t test. FIG. 13E illustrates CD11c, CD8, Iba1 staining in 9.5-month TE3-IgG and TE3-αIFNγ treated mice. Scale bar=50 μm. FIG. 13F depicts quantification of area covered by CD11c in 9.5-month TE3-IgG and TE3-αIFNγ mice. (TE3-IgG: n=12 and TE3-αIFNγ: n=12). *p=0.0344. Unpaired two-tailed Student's t test. FIG. 13G shows representative images of 9.5-month TE3-IgG and TE3-αIFNγ treated mouse brain sections stained with AT8 antibody. Scale bar=250 μm. FIG. 13H depicts p-Tau (AT8) covered area in 9.5-month TE3-IgG and TE3-αIFNγ treated mice. (TE3-IgG: n=12 and TE3-αIFNγ: n=12). *p=0.0122. Unpaired two-tailed Student's t test. FIG. 13I illustrates schematic representation of the timeline of PLX3397 treatment for microglia depletion. FIG. 13J illustrates Schematic representation of the timeline of anti-CD4 and anti-CD8 antibody treatment for T cell depletion.

FIG. 14A-14B shows flow plot and quantification of CD4⁺, CD8⁺ T cells in brain parenchyma, meninges and blood in IgG control or a-CD4 and a-CD8 treated mice. FIG. 14C displays microglia from brain parenchyma of TE4-IgG and TE4-aT treated mice assigned into 3 categories as visualized by UMAP plot. FIG. 14D illustrates bar plot showing the percentage of the 3 categories of microglia in TE4-IgG and TE4-aT mice. FIG. 14E depicts heat map showing representative functional genes specifically expressed in active microglia clusters in TE4-IgG and TE4-cxT mice. FIG. 14F shows quantification of nest-building behavior at 9.5 months age. (TE4-IgG: n=15 and TE4-cxT: n=21). *p=0.02 for IgG vs. cxT. Fisher's exact test. FIG. 14G displays quantification of total rearing at baseline levels of general exploratory behavior in 1 h. (TE4-IgG: n=10 and TE4-cxT: n=15). p=0.4363 for TE4-IgG vs. TE4-cxT. Unpaired two-tailed Student's t test. FIG. 14H shows quantification of total ambulations at baseline levels of locomotor activity levels in 1 h. (TE4-IgG: n=10 and TE4-cxT: n=15). p=0.0709. Mann-Whitney test. FIG. 14I displays schematic representation of fear conditioning behavioral paradigms.

FIG. 15A shows representative images of 9.5-month E4-IgG, E4-aT, TE4-IgG and TE4-aT mouse brain sections stained with Sudan black. Scale bar=1 mm. FIG. 15B-D display volumes of hippocampus, entorhinal/piriform cortex and posterior lateral ventricle in 9.5-month E4-IgG, E4-αT, TE4-IgG and TE4-αT mice. (E4-IgG: n=11; E4-αT: n=10; TE4-IgG: n=8 and TE4-αT: n=12). *p=0.0112, *p=0.0397, *p=0.0313 for TE4-IgG vs. TE4-αT comparing volumes of hippocampus, entorhinal/piriform cortex and posterior lateral ventricle, respectively. Unpaired two-tailed Student's t test. FIG. 15E illustrates Iba1 and CD3 staining in 9.5-month TE4-IgG and TE4-αT mice. Scale bar=20 μm. FIG. 15F shows P2ryl2, MHCII and Iba1 staining in 9.5-month TE4-IgG and TE4-αT mice. Scale bar=50 μm. FIG. 15G displays CD11c, CD8, Iba1 staining in 9.5-month TE4-IgG and TE4-αT mice. Scale bar=50 μm. FIG. 15H shows quantification of the number of CD3⁺ T cells in DG per 0.3 mm² in 9.5-month E4-IgG, E4-αT, TE4-IgG and TE4-αT mice. (E4-IgG: n=11; E4-αT: n=11; TE4-IgG: n=11 and TE4-αT: n=11). ***p=0.0004 for TE4-IgG vs. TE4-αT. Unpaired two-tailed Student's t test. FIG. 15i shows quantification of the number of CD8⁺ T cells in DG with 0.3 mm² in 9.5-month TE4-IgG and TE4-αT mice. (TE4-IgG: n=11 and TE4-αT: n=11). ***p=0.0002. Unpaired two-tailed Student's t test. FIG. 15J-M shows quantification of the covered areas of Iba1, P2ryl2, MHCII and CD11c in 9.5-month TE4-IgG and TE4-αT mice. (TE4-IgG: n=11 and TE4-αT: n=11). ***p=0.0002, *p=0.0229, ***p=0.0004, ***p=0.0002 for TE4-IgG vs. TE4-αT comparing area of Iba1, P2ryl2, MHCII and CD11c, respectively. Unpaired two-tailed Student's t test. FIG. 15N depicts quantification of the area covered by AT8 in DG per slice in 9.5-month TE4-IgG and TE4-αT mice. (TE4-IgG: n=11 and TE4-αT: n=11). ***p=0.0006. Unpaired two-tailed Student's t test. FIG. 15O depicts Four distinct p-Tau staining patterns were identified based on hippocampus staining patterns. **p=0.007 for four types distribution between TE4-IgG and TE4-αT. Fisher's exact test. FIG. 15P illustrates percentage of mice showed four p-Tau staining patterns in 9.5-months TE4-IgG and TE4-αT mice. FIG. 15Q displays quantification of NfL concentration in the plasma of 9.5-month E4-IgG, E4-αT, TE4-IgG and TE4-αT mice. (E4-IgG: n=11; E4-αT: n=11; TE4-IgG: n=11 and TE4-αT: n=11). *p=0.0398 for TE4-IgG vs. TE4-αT. Unpaired two-tailed Student's t test. FIG. 15R shows quantification of Y maze behavior in 8.5-month TE4-IgG and TE4-aT mice. (TE4-IgG: n=10 and TE4-αT: n=15). *p=0.0239 for TE4-IgG vs. TE4-αT. Unpaired two-tailed Student's t test. FIG. 15S illustrates quantification of total tone/shock pairing at day 1. (TE4-IgG: n=10 and TE4-αT: n=15). p=0.2152 for TE4-IgG vs. TE4-αT. Two-way ANOVA, with Bonferroni post hoc comparisons test. FIG. 15T shows quantification of freezing in response to contextual cue at day 2. (TE4-IgG: n=10 and TE4-αT: n=15). p=0.067 for TE4-IgG vs. TE4-αT. Two-way ANOVA, with Bonferroni post hoc comparisons test. FIG. 15U depicts quantification of freezing in response to auditory cue at day 3. (TE4-IgG: n=10 and TE4-αT: n=15). **p=0.0118 for TE4-IgG vs. TE4-αT. Two-way ANOVA, with Bonferroni post hoc comparisons test.

FIG. 16A depicts the gating strategy for sorting Foxp3+CD4+ Treg, Pd1+Foxp3+CD4+ Treg, Klrg1+ effector CD8+ T cells, PD-1+Tox1+CD8+ exhausted T cells in the brain parenchyma. FIGS. 16B-16E display quantification of T cell populations in the brain parenchyma of mice treated with IgG control and αPD-1 antibodies. FIG. 16F illustrate representative images of 9.5-month TE4-IgG and TE4-αPD-1 treated mouse brain sections stained with Sudan black. Scale bar=1 mm. FIGS. 16G-16H illustrate volumes of hippocampus, entorhinal/piriform cortex in 9.5-month TE4-IgG and TE4-αPD-1 treated mice. (TE4-IgG: n=14 and TE4-αPD: n=14). p=0.018 and 0.015 for TE4-IgG vs. TE4-αPD-1 in comparing the volumes of hippocampus, entorhinal/piriform cortex, respectively. FIG. 16I shows quantification of the area covered by AT8 in DG per slice in 9.5-month TE4-IgG vs. TE4-αPD-1 mice (TE4-IgG: n=14 and TE4-αPD: n=14). p=0.0009. Unpaired two-tailed Student's t test.

FIG. 17A shows representative images of 9.5-month-old P301S transgenic mouse hemibrain and P301S/Rag2KO mouse hemibrain. Brain sections are stained with Sudan black. FIGS. 17B-17C show the volumes of hippocampus (left panel), piriform and entorhinal cortex combined (piri-ent ctx) (right panel), and posterior lateral ventricle (middle panel) in 9.5-month-old WT, Rag2KO, P301S and P301S/Rag2KO male (FIG. 17B) and female (FIG. 17C) mice. Hippocampal and piriform/entorhinal cortex volumes are significantly greater (except in the piri-ent cortex in males) and ventricular volumes smaller in the P301S/Rag2KO vs. the P301S mice indicating less neurodegeneration in the absence of T cells and B cells. FIG. 17D shows representative images of 9.5-month-old P301S and P301S/Rag2KO mouse brain sections stained with AT8 to assess phosphorylated tau. FIGS. 17E-17F show quantification of the AT8 immunostained areas in the hippocampus of 9.5-month-old P301S and P301S/Rag2KO mice. The % area of the hippocampus covered with AT8 staining in 9.5-month-old P301S and P301S/Rag2KO male (FIG. 17E) and female (FIG. 17F) mice is shown. There is significantly less AT8 staining in the P301S/Rag2KO vs. P301S mice indicating less tau pathology in the absence of T and B cells. FIGS. 17G-17H show concentration of neurofilament light chain (NFL) in the plasma of 9.5-month-old WT, Rag2KO, P301S and P301S/Rag2KO male (FIG. 17G) and female (FIG. 17H) mice that was measured. NFL levels are significantly lower in the P301S/Rag2KO vs. the P301S mice indicating less axonal degeneration in the absence of T and B cells.

### DETAILED DESCRIPTION

The inventors have discovered, among other things, that disrupting the interaction between activated microglia and T cells that have infiltrated the brain parenchyma decreases the pathological neuroinflammation, as well as the brain damage, caused by aggregated tau accumulation. Certain aspects of the present disclosure, therefore, encompass methods of decreasing tau-mediated neurodegeneration in a subject in need thereof, methods of decreasing central nervous system (CNS) p-tau in a subject in need thereof, methods of reducing Nfl release from damaged axons in the brain of a subject, methods of reducing reactive microglia in the brain parenchyma of a subject, and methods of reducing brain atrophy in a tau positive subject. In each of these aspects, the subject is administered a composition comprising one or more agents that inhibit T-cell activation, decrease T-cell numbers, inhibit microglia activation, decrease microglia number, or any combination thereof.

### Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

An antibody, as used herein, refers to a complete antibody as understood in the art, i.e., consisting of two heavy chains and two light chains, and also to any antibody-like molecule that has an antigen-binding region, including, but not limited to, antibody fragments such as Fab′, Fab, F(ab′) 2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art. See, for instance U.S. Pat. No. 7,939,313, herein incorporated by reference in its entirety.

The term “tau” refers to a plurality of isoforms encoded by the gene MAPT (or homolog thereof), as well as species thereof that are C-terminally truncated in vivo, N-terminally truncated in vivo, post-translationally modified in vivo, or any combination thereof. As used herein, the terms “tau” and “tau protein” and “tau species” may be used interchangeably. In many animals, including but not limited to humans, nonhuman primates, rodents, fish, cattle, frogs, goats, and chicken, tau is encoded by the gene MAPT. In animals where the gene is not identified as MAPT, a homolog may be identified by methods well known in the art.

In humans, there are six isoforms of tau that are generated by alternative splicing of exons 2, 3, and 10 of MAPT. These isoforms range in length from 352 to 441 amino acids. Exons 2 and 3 encode 29-amino acid inserts each in the N-terminus (called N), and full-length human tau isoforms may have both inserts (2N), one insert (1 N), or no inserts (ON). All full-length human tau isoforms also have three repeats of the microtubule binding domain (called R). Inclusion of exon 10 at the C-terminus leads to inclusion of a fourth microtubule binding domain encoded by exon 10. Hence, full-length human tau isoforms may be comprised of four repeats of the microtubule binding domain (exon 10 included: R1, R2, R3, and R4) or three repeats of the microtubule binding domain (exon 10 excluded: R1, R3, and R4). Human tau may or may not be post-translationally modified. For example, it is known in the art that tau may be phosphorylated, ubiquinated, glycosylated, and glycated. Human tau also may or may not be proteolytically processed in vivo at the C-terminus, at the N-terminus, or at the C-terminus and the N-terminus. Accordingly, the term “human tau” encompasses the 2N3R, 2N4R, 1 N3R, 1 N4R, 0N3R, and 0N4R isoforms, as well as species thereof that are C-terminally truncated in vivo, N-terminally truncated in vivo, post-translationally modified in vivo, or any combination thereof. Alternative splicing of the gene encoding tau similarly occurs in other animals.

A disease associated with tau deposition in the brain is referred to herein as a “tauopathy”. The term “tau deposition” is inclusive of all forms pathological tau deposits including but not limited to neurofibrillary tangles, neuropil threads, and tau aggregates in dystrophic neurites. Tauopathies known in the art include, but are not limited to, progressive supranuclear palsy (PSP), dementia pugilistica, chronic traumatic encephalopathy, frontotemporal dementia and parkinsonism linked to chromosome 17, Lytico-Bodig disease, Parkinson-dementia complex of Guam, tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration (CBD), argyrophilic grain disease (AGD), frontotemporal lobar degeneration (FTLD), Alzheimer's disease (AD), Down syndrome with Alzheimer's disease, and frontotemporal dementia (FTD).

Tauopathies are classified by the predominance of tau isoforms found in the pathological tau deposits. Those tauopathies with tau deposits predominantly composed of tau with three MTBRs are referred to as “3R-tauopathies”. Pick's disease is a non-limiting example of a 3R-tauopathy. For clarification, pathological tau deposits of some 3R-tauopathies may be a mix of 3R and 4R tau isoforms with 3R isoforms predominant. Intracellular neurofibrillary tangles (i.e. tau deposits) in brains of subjects with Alzheimer's disease are generally thought to contain both approximately equal amounts of 3R and 4R isoforms. Those tauopathies with tau deposits predominantly composed of tau with four MTBRs are referred to as “4R-tauopathies”. PSP, CBD, and AGD are non-limiting examples of 4R-tauopathies, as are some forms of FTLD. Notably, pathological tau deposits in brains of some subjects with genetically confirmed FTLD cases, such as some V334M and R406W mutation carriers, show a mix of 3R and 4R isoforms.

A clinical sign of a tauopathy may be aggregates of tau in the brain, including but not limited to neurofibrillary tangles. Methods for detecting and quantifying tau aggregates in the brain are known in the art (e.g., tau PET using tau-specific ligands such as THK5317, THK5351, AV1451, PBB3, MK-6240, RO-948, PI-2620, GTP1, PM-PBB3, and JNJ64349311, JNJ-067, etc.).

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. Accordingly, a subject in need of treatment may or may not have any symptoms or clinical signs of disease.

The phrase “tau therapy” collectively refers to any imaging agent, therapeutic treatment, and/or a prophylactic or preventative measure contemplated for, or used with, subjects at risk of developing a tauopathy, or subjects clinically diagnosed as having a tauopathy. Non-limiting examples of imaging agents include functional imaging agents (e.g. fluorodeoxyglucose, etc.) and molecular imaging agents (e.g., Pittsburgh compound B, florbetaben, florbetapir, flutemetamol, radiolabeled tau-specific ligands, radionuclide-labeled antibodies, etc.). Non-limiting examples of therapeutic agents include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-A|3 antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRxO237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, (3-2 adrenergic receptor agonists, nicotinic acetylcholine receptor agonists, 5-HT2A inverse agonists, alpha-2c adrenergic receptor antagonists, 5-HT 1A and 1 D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

### I. Compositions

A composition of the present invention comprises one or more agents that disrupt the interaction between activated microglia and T cells that have infiltrated the brain parenchyma in subjects with aggregated tau accumulation, or subjects at risk thereof. In an aspect, a composition of the present invention comprises one or more agents that inhibit T-cell activation, decrease T-cell numbers, inhibit microglia activation, decrease microglia number, or any combination thereof.

### (a) Inhibit T-Cell Activation or Decrease T-Cell Number

In one embodiment, an agent that disrupts the interaction between activated microglia and T cells that have infiltrated the brain parenchyma in subjects with aggregated tau accumulation, or subjects at risk thereof, is an agent that inhibits T-cell activation or decreases T-cell number. In a preferred embodiment, the T-cell is not a regulatory T-cell. In another preferred embodiment, the T-cell is a proinflammatory T-cell. In some embodiments, the T-cell is a CD8+ cell. In other embodiments, the T-cell is a CD4+ cell.

Non-limiting examples of suitable agents may include fingolimid, JAK inhibitors such as Tofacitinib (Xeljanz), abrocitinib (Cibinqo), Baricitinib (Olumiant), Upadacitinib (Rinvoq), Peficitinib (Smyraf), Ruxolitinib (Jakafi), fedratinib (inrebic), pacritinib (vonjo), Deucravacitinib (Sotyktu), Filgotinib (Jyseleca), cerdulatinib, gandotinib, lestaurtinib, and momelotinib, antilymphocyte drugs such as OKT3, antithymocytegamma globulin (ATGAM), Daclizumab, and Basiliximab (anti IL2R), pan-T-cell antibodies such as anti-CD3 or anti-CD4/CD4a antibodies, biologic agents that interfere with T-cell helper signals, such as anti-CD40 receptor or anti-CD40 ligand (CD154), including blocking antibodies to CD40-CD40 ligand and CTLA4-Ig (e.g. Abatacept), and T-cell receptor antibodies (see, for instance, EP0340109).

In further non-limiting examples, suitable agents may include an antibody that binds to integrin α4β1 (very late antigen-4; VLA-4) or an antigen-binding fragment thereof. In some embodiments, the anti-VLA-4 antibody is an anti-CD49d antibody. In some embodiments, the anti-VLA-4 antibody specifically binds to CD49d of the VLA-4. In some embodiments, the anti-VLA-4 antibody is an anti-CD29 antibody. In some embodiments, the anti-VLA-4 antibody specifically binds to CD29 of the VLA-4. In some embodiments the anti-VLA-4 antibody is natalizumab or a biosimilar antibody thereof. Suitable agents may also include VLA-4 antagonist (e.g., inhibitor). In some embodiments, the VLA-4 antagonist is GW559090. In some embodiments, the VLA-4 antagonist is 7HP349.

In further non-limiting examples, suitable agents may include agents that block or reduce IFNgamma activity, such as IFNgamma inhibiting antibodies, or IFNgamma inhibiting compounds known in the art.

### (b) Inhibit Microglia Activation or Decrease Microglia Number

In one embodiment, an agent that disrupts the interaction between activated microglia and T cells that have infiltrated the brain parenchyma in subjects with tau-mediated neurodegeneration, or subjects at risk thereof, is an agent that inhibits microglia activation or decreases microglia number. In a preferred embodiment, the microglia cell is activated. In another preferred embodiment, the microglia is a disease-associated microglia. In yet another preferred embodiment, the microglia is an interferon-activated microglia.

Non-limiting examples of suitable agents may include CD11b-HSVTK, CD11b-HSVK^{mt}, PLX5622, BLZ945, PLX3397, GW2580, CX3CR1^{Cre}DTR, and LEC.

### (c) Combinations Thereof

A composition of the present invention comprises one or more agents that disrupt the interaction between activated microglia and T cells that have infiltrated the brain parenchyma in subjects with aggregated tau accumulation, or subjects at risk thereof. For instance, a composition may comprise one, two, three, four, five, six, seven, eight, nine, ten, or more than ten agents that disrupt the interaction between activated microglia and T cells that have infiltrated the brain parenchyma in subjects with aggregated tau accumulation, or subjects at risk thereof.

In certain compositions, a composition of the present invention may comprise an additional agent that treats a tauopathy. For instance, a composition of the present invention may comprise one or more agents detailed above, and an anti-amyloid antibody or antigen-binding fragment thereof. By way of non-limiting example, suitable anti-amyloid antibodies may include lecanemab and donanemab.

### (d) Formulations Thereof

The present disclosure also provides pharmaceutical compositions. A pharmaceutical composition comprises an agent described above as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

### II. Methods of the Disclosure

As detailed above, the present disclosure encompasses methods of decreasing tau-mediated neurodegeneration in a subject in need thereof, methods of decreasing central nervous system (CNS) p-tau in a subject in need thereof, methods of reducing Nfl release from axons in the brain of a subject, methods of reducing reactive microglia in the brain parenchyma of a subject, and methods of reducing brain atrophy in a tau positive subject.

### (a) Decreasing Tau-Mediated Neurodegeneration

One embodiment of the present disclosure encompasses a method of decreasing tau-mediated neurodegeneration in a subject in need thereof. Generally speaking, the method comprises administering a composition to disrupt the interaction between activated microglia and T cells that have infiltrated the brain parenchyma in subjects with tau-mediated neurodegeneration, or subjects at risk thereof.

Methods of measuring tau-mediated neurodegeneration are known in the art, and may include direct and indirect methods of measurement.

In one embodiment, the present disclosure encompasses a method of decreasing tau-mediated neurodegeneration in a subject in need thereof, the method comprising administering a composition comprising at least one agent that inhibits T-cell activation. In a more specific embodiment, the method comprises administering a composition comprising at least one agent that inhibits parenchymal infiltrated T cell activation. In some embodiments, the composition comprises at least one agent that inhibits T-cell activation and a second agent that inhibits parenchymal microglia activation or decreases parenchymal microglia numbers.

In another embodiment, the present disclosure encompasses a method of decreasing tau-mediated neurodegeneration in a subject in need thereof, the method comprising administering a composition comprising at least one agent that decreases parenchymal infiltrated T cells. In some embodiments, the composition comprises at least one agent that decreases parenchymal infiltrated T cells and a second agent that inhibits parenchymal microglia activation or decreases parenchymal microglia numbers.

In another embodiment, the present disclosure encompasses a method of decreasing tau-mediated neurodegeneration in a subject in need thereof, the method comprising administering a composition comprising at least one agent that inhibits parenchymal microglia activation. In some embodiments, the composition comprises at least one agent that inhibits inhibits parenchymal microglia activation and a second agent that inhibits Tcell activation or decreases T cell numbers.

In another embodiment, the present disclosure encompasses a method of decreasing tau-mediated neurodegeneration in a subject in need thereof, the method comprising administering a composition comprising at least one agent that decreases parenchymal microglia numbers. In some embodiments, the composition comprises at least one agent that decreases parenchymal microglia numbers and a second agent that inhibits Tcell activation or decreases Tcell numbers.

In the methods of decreasing tau-mediated neurodegeneration detailed above, a subject may be administered a composition detailed herein at, or shortly before, the time the tau pathology begins to lead to neuronal damage. Generally speaking, this occurs before cognitive decline in the subject. Methods of determining the stage of tau pathology in a subject are known in the art, and may include, for instance, measuring the levels of known biomarkers. In other embodiments, a subject may be administered a composition detailed herein after neuronal damage has occurred. For instance, by way of non-limiting example, a composition may be administered during very mild to moderate stages of clinical disease.

(b) Decreasing Central Nervous System p-Tau

One embodiment of the present disclosure encompasses a method of decreasing central nervous system p-tau in a subject in need thereof. Generally speaking, the method comprises administering a composition to disrupt the interaction between activated microglia and T cells that have infiltrated the brain parenchyma in subjects with tau-mediated neurodegeneration, or subjects at risk thereof.

Methods of measuring central nervous system p-tau are known in the art, and may include direct and indirect methods of measurement. In some embodiments, the amount of p-tau in a subject may be used to ‘stage’ the subject. In such embodiments, a method of the present disclosure encompasses moving a subject from a higher stage to a lower stage. The method comprises administering a composition to disrupt the interaction between activated microglia and T cells that have infiltrated the brain parenchyma in subjects with tau-mediated neurodegeneration, or subjects at risk thereof.

In one embodiment, the present disclosure encompasses a method of decreasing central nervous system p-tau in a subject in need thereof, the method comprising administering a composition comprising at least one agent that inhibits T-cell activation. In a more specific embodiment, the method comprises administering a composition comprising at least one agent that inhibits parenchymal infiltrated T cell activation. In some embodiments, the composition comprises at least one agent that inhibits T-cell activation and a second agent that inhibits parenchymal microglia activation or decreases parenchymal microglia numbers.

In another embodiment, the present disclosure encompasses a method of decreasing central nervous system p-tau in a subject in need thereof, the method comprising administering a composition comprising at least one agent that decreases parenchymal infiltrated T cells. In some embodiments, the composition comprises at least one agent that decreases parenchymal infiltrated T cells and a second agent that inhibits parenchymal microglia activation or decreases parenchymal microglia numbers.

In another embodiment, the present disclosure encompasses a method of decreasing central nervous system p-tau in a subject in need thereof, the method comprising administering a composition comprising at least one agent that inhibits parenchymal microglia activation. In some embodiments, the composition comprises at least one agent that inhibits inhibits parenchymal microglia activation and a second agent that inhibits Tcell activation or decreases T cell numbers.

In another embodiment, the present disclosure encompasses a method of decreasing central nervous system p-tau in a subject in need thereof, the method comprising administering a composition comprising at least one agent that decreases parenchymal microglia numbers. In some embodiments, the composition comprises at least one agent that decreases parenchymal microglia numbers and a second agent that inhibits Tcell activation or decreases Tcell numbers.

In the methods of decreasing central nervous system p-tau detailed above, a subject may be administered a composition detailed herein at, or shortly before, the time the tau pathology begins to lead to neuronal damage. Generally speaking, this occurs before cognitive decline in the subject. Methods of determining the stage of tau pathology in a subject are known in the art, and may include, for instance, measuring the levels of known biomarkers. In other embodiments, a subject may be administered a composition detailed herein after neuronal damage has occurred. For instance, by way of non-limiting example, a composition may be administered during very mild to moderate stages of clinical disease.

### (c) Reducing Brain Atrophy in a Tau Positive Subject

One embodiment of the present disclosure encompasses a method of reducing brain atrophy in a tau positive subject in need thereof. Generally speaking, the method comprises administering a composition to disrupt the interaction between activated microglia and T cells that have infiltrated the brain parenchyma in subjects with tau-mediated neurodegeneration, or subjects at risk thereof.

Methods of measuring brain atrophy in a tau positive subject are known in the art, and may include direct and indirect methods of measurement.

In one embodiment, the present disclosure encompasses a method of reducing brain atrophy in a tau positive subject in need thereof, the method comprising administering a composition comprising at least one agent that inhibits T-cell activation. In a more specific embodiment, the method comprises administering a composition comprising at least one agent that inhibits parenchymal infiltrated T cell activation. In some embodiments, the composition comprises at least one agent that inhibits T-cell activation and a second agent that inhibits parenchymal microglia activation or decreases parenchymal microglia numbers.

In another embodiment, the present disclosure encompasses a method of reducing brain atrophy in a tau positive subject in need thereof, the method comprising administering a composition comprising at least one agent that decreases parenchymal infiltrated T cells. In some embodiments, the composition comprises at least one agent that decreases parenchymal infiltrated T cells and a second agent that inhibits parenchymal microglia activation or decreases parenchymal microglia numbers.

In another embodiment, the present disclosure encompasses a method of reducing brain atrophy in a tau positive subject in need thereof, the method comprising administering a composition comprising at least one agent that inhibits parenchymal microglia activation. In some embodiments, the composition comprises at least one agent that inhibits inhibits parenchymal microglia activation and a second agent that inhibits Tcell activation or decreases T cell numbers.

In another embodiment, the present disclosure encompasses a method of reducing brain atrophy in a tau positive subject in need thereof, the method comprising administering a composition comprising at least one agent that decreases parenchymal microglia numbers. In some embodiments, the composition comprises at least one agent that decreases parenchymal microglia numbers and a second agent that inhibits Tcell activation or decreases Tcell numbers.

In the methods of reducing brain atrophy in a tau positive subject detailed above, a subject may be administered a composition detailed herein at, or shortly before, the time the tau pathology begins to lead to neuronal damage. Generally speaking, this occurs before cognitive decline in the subject. Methods of determining the stage of tau pathology in a subject are known in the art, and may include, for instance, measuring the levels of known biomarkers. In other embodiments, a subject may be administered a composition detailed herein after neuronal damage has occurred. For instance, by way of non-limiting example, a composition may be administered during very mild to moderate stages of clinical disease.

(d) Reducing Nfl Release from Axons in the Brain of a Subject

One embodiment of the present disclosure encompasses a method of reducing Nfl release from axons in the brain of a subject in need thereof. Generally speaking, the method comprises administering a composition to disrupt the interaction between activated microglia and T cells that have infiltrated the brain parenchyma in subjects with tau-mediated neurodegeneration, or subjects at risk thereof.

Methods of measuring Nfl release from axons in the brain of a subject are known in the art, and may include direct and indirect methods of measurement.

In one embodiment, the present disclosure encompasses a method of reducing Nfl release from axons in the brain of a subject in need thereof, the method comprising administering a composition comprising at least one agent that inhibits T-cell activation. In a more specific embodiment, the method comprises administering a composition comprising at least one agent that inhibits parenchymal infiltrated T cell activation. In some embodiments, the composition comprises at least one agent that inhibits T-cell activation and a second agent that inhibits parenchymal microglia activation or decreases parenchymal microglia numbers.

In another embodiment, the present disclosure encompasses a method of reducing Nfl release from axons in the brain of a subject in need thereof, the method comprising administering a composition comprising at least one agent that decreases parenchymal infiltrated T cells. In some embodiments, the composition comprises at least one agent that decreases parenchymal infiltrated T cells and a second agent that inhibits parenchymal microglia activation or decreases parenchymal microglia numbers.

In another embodiment, the present disclosure encompasses a method of reducing Nfl release from axons in the brain of a subject in need thereof, the method comprising administering a composition comprising at least one agent that inhibits parenchymal microglia activation. In some embodiments, the composition comprises at least one agent that inhibits inhibits parenchymal microglia activation and a second agent that inhibits Tcell activation or decreases T cell numbers.

In another embodiment, the present disclosure encompasses a method of reducing Nfl release from axons in the brain of a subject in need thereof, the method comprising administering a composition comprising at least one agent that decreases parenchymal microglia numbers. In some embodiments, the composition comprises at least one agent that decreases parenchymal microglia numbers and a second agent that inhibits Tcell activation or decreases Tcell numbers.

In the methods of reducing Nfl release from axons in the brain of a subject detailed above, a subject may be administered a composition detailed herein at, or shortly before, the time the tau pathology begins to lead to neuronal damage. Generally speaking, this occurs before cognitive decline in the subject. Methods of determining the stage of tau pathology in a subject are known in the art, and may include, for instance, measuring the levels of known biomarkers. In other embodiments, a subject may be administered a composition detailed herein after neuronal damage has occurred. For instance, by way of non-limiting example, a composition may be administered during very mild to moderate stages of clinical disease.

### (e) Reducing Reactive Microglia in the Brain Parenchyma

One embodiment of the present disclosure encompasses a method of reducing reactive microglia in the brain parenchyma in a subject in need thereof. Generally speaking, the method comprises administering a composition to disrupt the interaction between activated microglia and T cells that have infiltrated the brain parenchyma in subjects with tau-mediated neurodegeneration, or subjects at risk thereof.

Methods of measuring reactive microglia in the brain parenchyma are known in the art, and may include direct and indirect methods of measurement.

In one embodiment, the present disclosure encompasses a method of reducing reactive microglia in the brain parenchyma in a subject in need thereof, the method comprising administering a composition comprising at least one agent that inhibits T-cell activation. In a more specific embodiment, the method comprises administering a composition comprising at least one agent that inhibits parenchymal infiltrated T cell activation. In some embodiments, the composition comprises at least one agent that inhibits T-cell activation and a second agent that inhibits parenchymal microglia activation or decreases parenchymal microglia numbers.

In another embodiment, the present disclosure encompasses a method of reducing reactive microglia in the brain parenchyma in a subject in need thereof, the method comprising administering a composition comprising at least one agent that decreases parenchymal infiltrated T cells. In some embodiments, the composition comprises at least one agent that decreases parenchymal infiltrated T cells and a second agent that inhibits parenchymal microglia activation or decreases parenchymal microglia numbers.

In another embodiment, the present disclosure encompasses a method of reducing reactive microglia in the brain parenchyma in a subject in need thereof, the method comprising administering a composition comprising at least one agent that inhibits parenchymal microglia activation. In some embodiments, the composition comprises at least one agent that inhibits inhibits parenchymal microglia activation and a second agent that inhibits Tcell activation or decreases T cell numbers.

In another embodiment, the present disclosure encompasses a method of reducing reactive microglia in the brain parenchyma in a subject in need thereof, the method comprising administering a composition comprising at least one agent that decreases parenchymal microglia numbers. In some embodiments, the composition comprises at least one agent that decreases parenchymal microglia numbers and a second agent that inhibits Tcell activation or decreases Tcell numbers.

In the methods of reducing reactive microglia in the brain parenchyma detailed above, a subject may be administered a composition detailed herein at, or shortly before, the time the tau pathology begins to lead to neuronal damage. Generally speaking, this occurs before cognitive decline in the subject. Methods of determining the stage of tau pathology in a subject are known in the art, and may include, for instance, measuring the levels of known biomarkers. In other embodiments, a subject may be administered a composition detailed herein after neuronal damage has occurred. For instance, by way of non-limiting example, a composition may be administered during very mild to moderate stages of clinical disease.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1: Methods

### Animals

Human ApoE knock-in mice, ApoE3^{flox/flox} and Apo E4^{flox/flox} (E3 and E4, respectively), were generated by replacing the mouse genomic sequence from the translation initiation codon in exon2 to the termination codon in exon4 with its human counterparts flanked by loxP sites52. P301S tau transgenic mice (Jax, #008169) on C57BL/6 background were crossed to human ApoE KI or ApoE KO mice to generate P301S/E3 (TE3), P301S/E4 (TE4), and P301S/EKO (TEKO) mice respectively. All tau transgenic mice involved in the final analysis were obtained from the same generation. A/PE4 and 5×FADE4 mice have been previously described. Littermates of the same sex were randomly assigned to experimental groups. All animal procedures and experiments were performed under guidelines approved by the Institutional Animal Care and Use committee (IACUC) at Washington University School of Medicine.

### Human AD Tissues

All participants gave prospective pre-mortem written consent for their brain to be banked and used for research. Patient demographics can be found in Table 1.

TABLE 1
Human AD patient sample list

| ID | Age | Sex | ApoE | Braak | PMI |
|---|---|---|---|---|---|
| 1 | 70 | F | E4/E4 | I | 2 |
| 2 | 78 | M | E2/E2 | I | 1.66 |
| 3 | 81 | F | E4/E4 | II | 3 |
| 4 | 80 | F | E4/E4 | VI | 1.33 |
| 5 | 68 | F | E2/E2 | VI | 3.08 |
| 6 | 80 | M | E3/E3 | VI | 2.16 |
| 7 | 83 | M | E4/E4 | VI | 2.83 |
| 8 | 89 | F | E3/E3 | VI | 2.7 |
| 9 | 90 | F | E3/E3 | VI | 2.53 |
| 10 | 89 | F | E4/E4 | VI | 2.5 |

### Volumetric Analysis

The left hemi-brain of each mouse was fixed by 4% paraformaldehyde for 24 h at 4° C. and then placed in 30% sucrose at 4° C. overnight. Serial free-floating coronal sections were cut from the rostral crossing of the corpus callosum to the caudal end of the hippocampus at 50 μm on a Lecia SM2010 microtome. Brain sections (spaced 300 μm apart) from bregma −1.3 mm to −3.1 mm were mounted for volumetric analysis. All mounted sections were stained with 0.1% Sudan black (Sigma, 199664-25G) in 70% ethanol at RT for 20 min, washed in 70% ethanol for 50s, 3 times. The sections were washed in Milli-Q water 3 times and covered with Floromount-G (Southern Biotech, 0100-01). Slides were scanned using Hamamatsu's NanoZoomer microscope at 20× magnification. Hippocampus, Ent/Piri cortex and ventricles were traced using NDP viewer. The volume was calculated using formula: volume=(sum of area)×0.3 mm.

### Immunohistochemistry

Two sections from each mouse (300 μm apart), corresponding approximately to bregma coordinates −1.4 mm, −1.7 mm were used for p-tau staining. Brain sections were washed in Tris-buffered saline (TBS) buffer for 3 min followed by incubation in 0.3% hydrogen peroxide in TBS for 10 min at RT. After 3 times washing in TBS, sections were blocked by 3% milk in TBS with 0.25% Triton X-100 (TBSX) for 1 h at RT followed by incubation with AT8-biotinylated antibody (Thermo Scientific, MN1020B) overnight at 4° C. The next day, after 3 times washing in TBS, the slices were developed by VECTASTAIN Elite ABC-HRP kit (Vector laboratories, PK-6100) following the manufacturer's instructions. Slides were covered by cytoseal60 (Thermo Scientific, 8310-4) and scanned using a Hamamatsu NanoZoomer microscope at 20× magnification. Images were analyzed in Image J.

For immunofluorescent staining, two sections (bregma −2.0 mm and −2.3 mm) from each mouse were used. The sections were washed in TBS 3 times, permeabilized with 0.25% TBSX for 10 min, followed by blocking with 3% BSA in 0.25% TBSX for 1 h at RT. Sections were incubated in primary antibodies overnight at 4° C. The next day, sections were washed in TBS and incubated with corresponding fluorescence-labeled secondary antibodies for 1.5 h at RT. The slices were washed and mounted in prolong gold antifade mounting media (Invitrogen, P36930). Primary antibodies were as follows: CD3 (Novus, NB600-1441, 1:200), Iba1 (Wako, 019-19741, 1:2000; Abcam, ab5076, 1:500), AT8 (Invitrogen, MN1020B, 1:500), Aβ (Homemade, HJ3.4B, 1:1000), P2ryl2 (Gift from Butovsky lab, 1:2000), NeuN (Abcam, ab177487, 1:1000), MBP (Abcam, ab7349, 1:500), MHCII (Biolgend, 107650, 1:200), X34 (Sigma, 1954-25 MG, 10 mM in DMSO stock, 1:5000), CD206 (Bio-Rad, MCA2235, 1:300), Hoechst (sigma, 94403, 1:5000). Secondary antibodies were highly cross-adsorbed with Alexa Fluor 568 (Thermo Fisher, 1:500); Images were acquired on a Zeiss LSM800 microscope. Antibody-fluorophores covered area and numbers were analyzed by ImageJ. 3D construction was performed using Imaris software. CD3, IBA1, CD8 and CD11c were labeled and detected with fluorophores using surface area function.

### PLX3397 Formulation and Supplement

PLX3397 was purchased from SelleckChem. PLX3397 was formulated in the AlN-76A (Research Diet) at a concentration of 400 mg/kg chow. E4 and TE4 mice were treated with PLX for 4 weeks for microglial acute depletion from 8.5 to 9.5 months of age.

### IFN-γ Treatment

For blocking IFN-γ signaling, mice were i.p. injected with 100 mg/kg body weight with either control IgG (Leinco, P376) or anti-mouse IFN-γ (Leinco, clone H22, I-1190) antibodies every 5 days from 7.5 to 9.5 months of age.

### Anti-PD-1 Treatment

For blocking PD-1/PD-L1 signaling chronically, mice were i.p. injected with 500 μg anti-PD-1 antibody (BioXCell, BP0146) every 5 days from 8 to 9.5 months of age. IgG (BioXCell, BP0089) isotype control was administered at the same frequency and dosage. Brains were collected for flow cytometry assessment of T cell populations. To characterize the T cell populations with anti-PD1 treatment, mice were acutely treated with 500 μg anti-PD1 or IgG every 2 days. After perfusion, brain was isolated for single cell for flow cytometry. Intracellular staining for transcription factors was performed using eBioscience FOXP3/Transcription Factor Kit (Ref. 00-5523-00) per manufacturer's instructions. In brief, cells were stained with LIVE/DEAD Fixable Aqua Dead Cell Stain Kit (Invitrogen, Ref. L34966A) for 5 min and then incubated with surface antibody mix and TruStain FcX PLUS (anti-mouse CD16/CD32, Clone S17011E, Biolegend, Ref. 156604, 1:200) for 1 h at room temperature. After cell-surface staining, cells were fixed, permeabilized, and incubated with intracellular antibody mix overnight at 4° C. Flow cytometry analysis was performed on a BD Symphony A3. The following antibodies were used. Biolegend: CD45.2 (104), CD4 (GK1.5), Pd1 (29F.1A12), Klrg1 (2F1/KLRG1). BD: CD3e (1452C11), CD8a (53-6.7). Invitrogen: Foxp3 (FJK-16s), Tox (TXRX10).

### T Cell Depletion

For the depletion of CD4⁺ and CD8⁺ T cells, mice were i.p. injected with 500 μg anti-CD4 (BioXCell, BP0003-1) and anti-CD8 antibody (BioXCell, BP0061) every 5 days from 6 to 9.5 months of age or for memory related behavioral experiments from 6 to 8.5 months of age. IgG (BioXCell, BP0090) isotype control was administered at the same frequency and dosage. To characterize the depletion efficiency, mice were acutely treated with 500 μg anti-CD4, or anti-CD8 or IgG. Brain, meninges and blood were extracted for single cell analysis followed by flow cytometry assessment of CD4⁺ and CD8⁺ T cell populations.

### Brain Extraction

Mouse cortex tissue was weighed and homogenized using a pestle with 10 μl buffer/1 mg tissue in chilled lysis buffer (Thermo Scientific, 78503). After centrifugation at 20,000 g for 10 min at 4° C., the supernatant was saved, and protein concentration was measured by micro BCA protein assay kit (Thermo Scientific, 23235) before multiplex immunoassay (Thermo Scientific).

### Nest-Building Behavior

Group-housed mice were switched to individual housing in the week of assessment at 9.5 months. Pre-weighted nestlet was provided in each cage. After an overnight housing, the remaining nestlet was weighted. The 5-point scale system was included and given based on percentage of remaining nesting material and shredded conditions. Score 1: nestlet >90% untorn; score 2:50-90% of nestlet is untorn; score 3: 10%-50% of nestlet is untorn; score 4: nestlet <10% untorn, but nest is flat and uncompact; score 5: nest is compact and nest wall is higher than the mouse for >50% of its circumference.

### General Design of Behavioral Tests

TE4 male mice were treated with IgG or with anti-CD4 and anti-CD8 antibodies for T cell depletion from 6 to 8.5 months of age. They were then tested for behavioral differences. Following 1 week habituation and handling in the Washington University Animal Behavior Core, mice were evaluated on 1 hour locomotor activity, spontaneous alternation in a Y maze, and fear conditioning. All tests were conducted during the light phase of the light/dark cycle. Behavioral testers were blind to the treatment group.

### One-Hour Locomotor Activity and Open-Field Behavior Test

To evaluate general activity levels and possible alterations in emotionality, mice were evaluated over a 1-h period in transparent (47.6×25.4×20.6 cm high) polystyrene enclosures. Each cage was surrounded by a frame containing a 4×8 matrix of photocell pairs, the output of which was fed to an on-line computer (Hamilton-Kinder, LLC, Poway, CA). The system software (Hamilton-Kinder, LLC) was used to define a 33×11 cm central zone and a peripheral or surrounding zone that was 5.5 cm wide with the sides of the cage being the outermost boundary. This peripheral area extended along the entire perimeter of the cage. Variables that were analyzed included the total number of ambulations and rearing on hindlimbs, as well as the number of entries, the time spent, and the distance traveled in the center area as well as the distance traveled in the periphery surrounding the center.

### Spontaneous Alternation Y-Maze

Testing was conducted according to previously published procedures. Briefly, this involved placing a mouse in the center of a Y-maze that contained three arms that were 10.5 cm wide, 40 cm long and 20.5 cm deep where an arm was oriented at 120° with respect to each successive other arm. Mice were allowed to explore the maze for 10 min and entry into an arm was scored only when the hindlimbs had completely entered the arm. An alternation was defined as any three consecutive choices of three different arms without re-exploration of a previously visited arm. Dependent variables included the number of alternations and arm entries along with the percentage of alternations, which was determined by dividing the total number of alternations by the total number of entries minus 2, then multiplying by 100.

### Conditioned Fear

A previously described protocol (Yuede, C. M. et al. PLoS One 5, e11374, 2010) was used to train and test mice using two clear-plastic conditioning chambers (26×18×18 cm high) (Med-Associates, St. Albans, VT) which were easily distinguished by different olfactory, visual, and tactile cues present in each chamber. On day 1, each mouse was placed into the conditioning chamber for 5 min and freezing behavior was quantified during a 2 min baseline period. Freezing (no movement except that associated with respiration) was quantified using FreezeFrame image analysis software (Actimetrics, Evanston, IL) which allows for simultaneous visualization of behavior while adjusting for a “freezing threshold” during 0.75 s intervals. After baseline measurements, a conditioned stimulus (CS) consisting of an 80 dB tone (white noise) was presented for 20 sec followed by an unconditioned stimulus (US) consisting of a 1 s, 1.0 mA continuous foot shock. This tone-shock (T/S) pairing was repeated each minute over the next 2 min, and freezing was quantified after each of the three tone-shock pairings. Twenty-four hours after training, each mouse was placed back into the original conditioning chamber to test for fear conditioning to the contextual cues in the chamber. This involved quantifying freezing over an 8 min period without the tone or shock being present. Twenty-four hours later, the mice were evaluated on the auditory cue component of the conditioned fear procedure, which included placing each mouse into the other chamber containing distinctly different cues. Freezing was quantified during a 2 min “altered context” baseline period as well as over a subsequent 8 min period during which the auditory cue (CS) was presented. Shock sensitivity was evaluated following completion of the conditioned fear test.

### NfL Concentration

Plasma NfL concentration was measured with NF-Light Simoa Assay Advantage kit, Quanterix.

### Single Cell Isolation

Mechanical dissociation was performed using a previously described method. Briefly, mice were perfused with pre-chilled PBS to fully remove blood contamination. Hippocampus and cortex were dissected followed by Dounce homogenization. Cell suspensions were then passed through Percoll density centrifugation to remove myelin and debris. The cell pellets were washed with 0.5% BSA for analysis or collection. For meninges, meninges were peeled intact from the skullcap using fine forceps and prepared for single cell analysis. Briefly, meninges were mashed through a cell strainer, using a sterile syringe plunger and washed in 0.5% BSA.

### Flow Cytometry for Single Cell

All steps were performed on ice or using pre-chilled centrifuge set to 4° C. Single cell suspensions were incubated with anti-CD16/32 (Fc block; Bio legend) for 5 min then fluorescently conjugated antibodies were added for 20 min. After washing, samples were collected by 300 g followed by a 5 min spin down and suspended in 5% BSA with PI for live/Dead selection before sorting. Cells were sorted using FACsAria II (BD Bioscience).

### Single Cell Immune Sequencing

After counting and analyzing single cell integrity, 8,000-16,000 individual single cells per sample were loaded onto a 10× Genomics Chromium platform for Gel Beads-in-emulsion (GEM) and cDNA generation carrying cell- and transcript-specific barcodes and sequencing libraries constructed using the Chromium Single Cell 5′library & Gel Bead Kit V2. Libraries were sequenced on the Illumina NovaSeq6000.

### Single Cell Data Processing and TCR Analysis

Alignment, barcode assignment, and UMI counting with Cell Ranger (v6.1.1) were used for preparation of count matrices for gene expression library. For alignment, a custom mouse genome (GRCm38) containing human sequences for APOE, PSEN1, APP, MAPT genes was used as a reference.

Barcodes in all samples that were considered to represent noise and low-quality cells were filtered out using knee-inflection strategy available in default CellRanger analysis. For downstream, Seurat package (version 4.0.4) was used, genes which express in less than three cells additionally filtered from expression matrices. The mitochondrial genes fraction was calculated for every cell, and cells with a mitochondrial fraction more than highest confidence interval for scaled mitochondrial percentage were filtered out which results in removal of the cells with mitochondrial percentage more than 20%. Additionally, cut off with log 10 (number of unique expressed genes) as 2.5 was used for removing the cells from both CD45^{High} and CD45^{Total} parenchyma cells, and 2 was used as a threshold for the cells from meninges.

Doublets have been excluded based on the co-expression of the canonical cell-type specific genes.

Each sample was normalized using SCTransform function with mitochondrial content as a variable to regress out in a second non-regularized linear regression. For integration aims, variable genes across the samples were identified by SelectIntegrationFeatures function with the number of features equal to 2000. Then the object was prepared for integration (PrepSCTIntegration function), the anchors were found (FindIntegrationAnchors function) and the samples were integrated into the whole object (IntegrateData function).

The principal component analysis was used for dimensionality reduction, and the first 20 principal components (PCs) were used further to generate uniform manifold approximation and projection (UMAP) dimensionality reduction by RunUMAP function. Clustering procedure was performed by FindNeighbors and FindClusters with a range of resolutions (from 0.2 to 1.0 with 0.2 as a step) and the first 20 PCs as input.

The object covering all cells was subsetted into T-cell, microglia and myeloid specific sub-objects based on expression of canonical gene markers. Additionally, the T-cells object was split into CD4⁺ and CD8⁺ cells. Then, all objects passed through the iterative process of quality control with doublets removal and exclusion of the cell types which have no relevant markers and contained high mitochondrial content as well as poor coverage (all filters are object-specific).

Cell Ranger's vdj workflow was used for TCR data analysis. Non-canonical T cells (such as gamma-delta T cells and natural killer T cells) as well as T cells with inappropriate combinations of alpha/beta chains were removed. Then, all barcodes were assigned to two populations based on CD4 and CD8 gene expression. The Gini coefficient was calculated using the immunarch package in order to estimate the clonal diversity among samples.

Trajectory analysis was done using a slingshot container available at dynverse package with normalized count matrices with barcodes assigned to microglia as input data as well as cells assigned to CD8⁺ T cells.

Interaction analysis was implemented using the CellChat package (v. 1.1.3) with the “Cell-Cell Contact” database. As input data, microglia, CD4 and CD8 T cells from E4 genotype and microglia, CD4⁺ and CD8⁺ T cells from TE4 genotype were used. Following the CellChat vignette, CellChat objects were prepared (createCellChat), overexpressed genes and interactions were identified (identifyOverExpressedGenes, identifyOverExpressedInteractions functions), communication probabilities were estimated (computeCommunProb, filterCommunication, computeCommunProbPathway functions), and network analysis (aggregateNet, netAnalysis_computeCentrality functions) was performed. The genotype-specific as well as genotype-common ligand-receptor pairs were identified (netVisual_bubble function). The number of interactions were evaluated using netVisual_circle function.

### Microglia Antigen Presentation In Vitro Assay

C57BL/6-Tg (TcraTcrb) 1100 Mb/J (OT-I) (Jax, #003831), B6.SJL-Ptprc^{a} Pepc^{b}/BoyJ (B6. CD45.1) (Jax, #002014) were from Jackson laboratory. OT-1.CD45.1/2 mice were generated by crossing OT-1 and B6.CD45.1 for one generation. 8-12 weeks old mice were used for the experiment.

Flow cytometry and cell sorting were completed on a FACS CantoII or FACS AriaII instrument and analyzed using Flowjo analysis software. Staining was performed at 4° C. in the presence of Fc block (2.4G2; Leinco) in magnetic-activated cell-sorting (MACS) buffer (0.5% BSA, 2 mM EDTA in PBS). The following antibodies were used. Biolegend: CD45 (30-F11), CD11b (M1/70), I-A/I-E (M5/114.15.2), CD3 (145-2C11), Vβ5 (MR9-4), Va2 (B20.1), CD45.2 (104), CD45.1 (A20), CD44 (IM7), CD8a (53-6.7), CD62L (MEL-14), CD19 (6D5), Ly6C (HK1.4), XCR1 (ZET), CD40 (3/23), CD80 (16-10A1), CD86 (GL-1), Sirpa (P84), NK1.1 (PK136). Invitrogen: CD11c (N418), Foxp3 (FJK-16s). BD Pharmingen: CD45RA (14.8) CellTrace Violet Cell Proliferation Kit (Thermo Fisher, C34571).

To prepare single cell suspensions of lymphocytes from spleen, spleens were harvested from the mice and smashed through 70 micron cell strainer with a plunger. To isolate APCs, spleens were chopped into small pieces and digested at 37° C. for 45 min with buffer containing 0.28 U/ml Liberase™ (Roche), 100 U/ml Hyaluronidase (Sigma), and 50U/ml DNase I in RMPI1640. Single cell suspensions were pelleted down by centrifugation at 1,500 rpm for 5 min and the cells were resuspended in 5 ml red blood cell lysis buffer (ACK buffer: 150 mM ammonium chloride, 10 mM potassium bicarbonate, and 0.1 mM EDTA) at room temperature for 2 min. The reaction was stopped by adding 1 ml of FBS and cells were passed through 70 micron strainer one more time. Cells were pelleted down and ~5×10⁶ cells were used for staining.

To enrich OT-1 CD8⁺ T cells and dendritic cells (DCs), CD8α+ T Cell Isolation Kit (Miltenyi Biotec) and CD11c beads (Miltenyi Biotec) were used for column-based enrichment. OT-1 naïve CD8⁺ T cells were sorted followed by CD45⁺CD3⁺CD8⁺ TCRVβ5⁺ TCRVα2⁺CD62L⁺CD44^{low} cells; Microglia were sorted followed by CD45^{low}CD11b⁺ cells; DCs were sorted as CD45⁺CD11c⁺MHC-II^{high} cells.

After sorting the microglia, DCs, and OT-1 T cells, CellTrace Violet labeled 25,000 T cells were co-cultured with 20,000 microglia or DCs for 3 days in U-bottom 96-well plate. A serial dilution of ovalbumin starting from 1000 μg/ml (2× dilution) was made and added into the wells. For microglia/OT-1 co-culture, two doses of IFNγ (100 ng/ml and 1000 ng/ml) were added at the same time. 3 days after, cells were analyzed by flow cytometry for T cell proliferation.

### Statistics

Statistical analysis was performed using Prism. Difference between groups were evaluated by Student's t test, one-way or two-way ANOVA followed by post hoc tests. For conditioned fear behavior, two-way ANOVA followed by Bonferroni test. Data expressed as mean±s.e.m. ***p<0.0001, **p<0.001, *p<0.05, n.s. no significant difference.

### Example 2: Increased T Cells with Tau Pathology

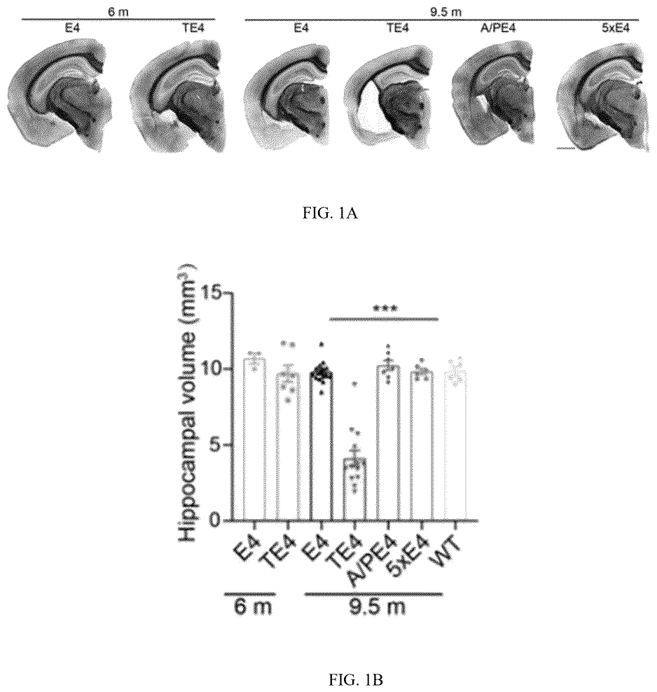

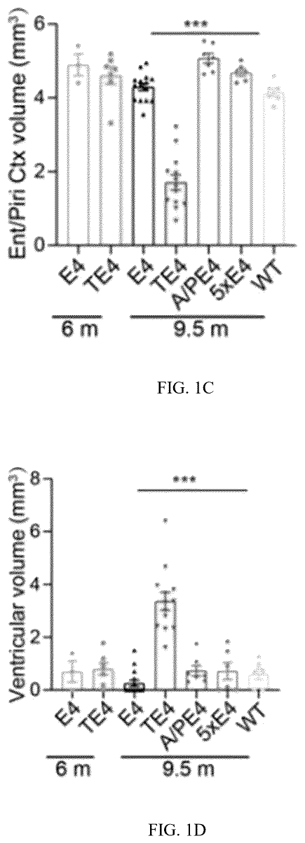

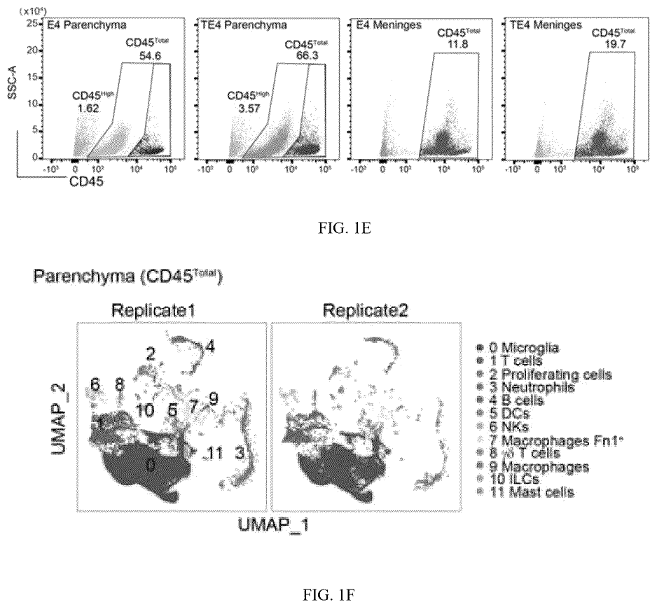

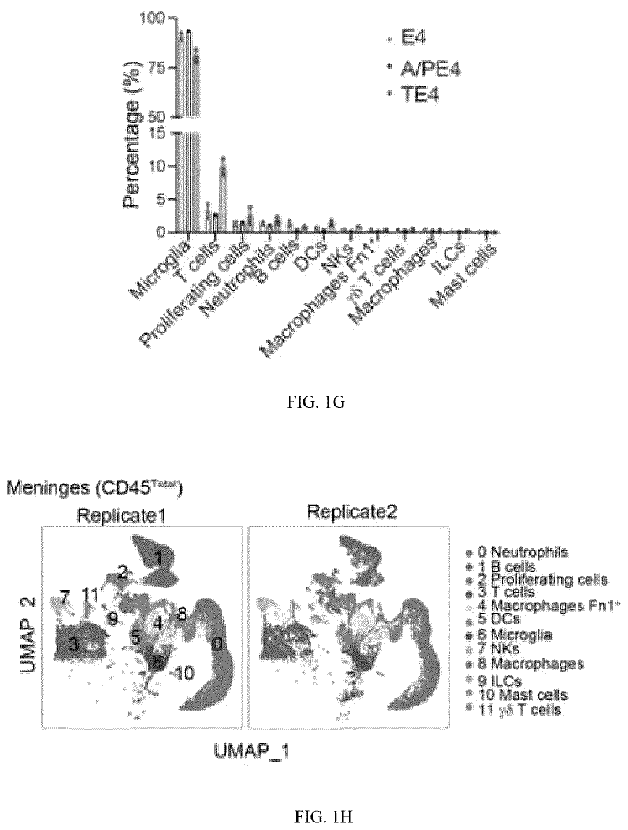

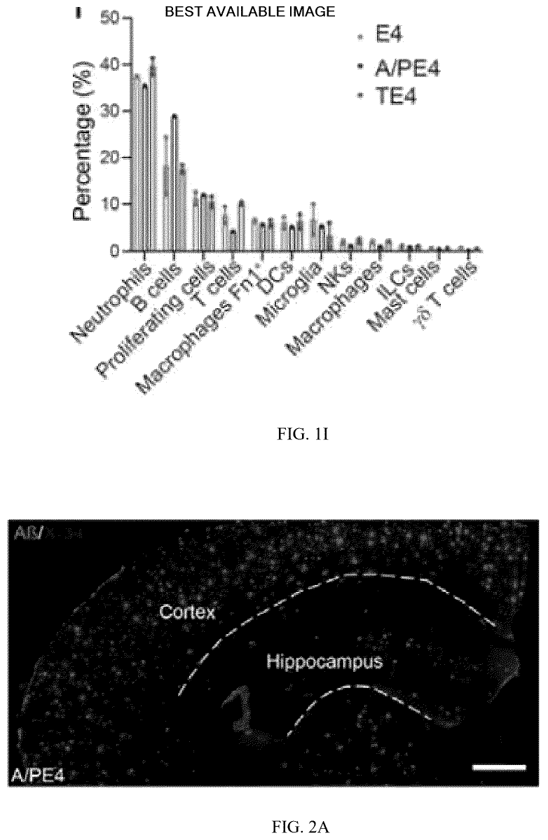

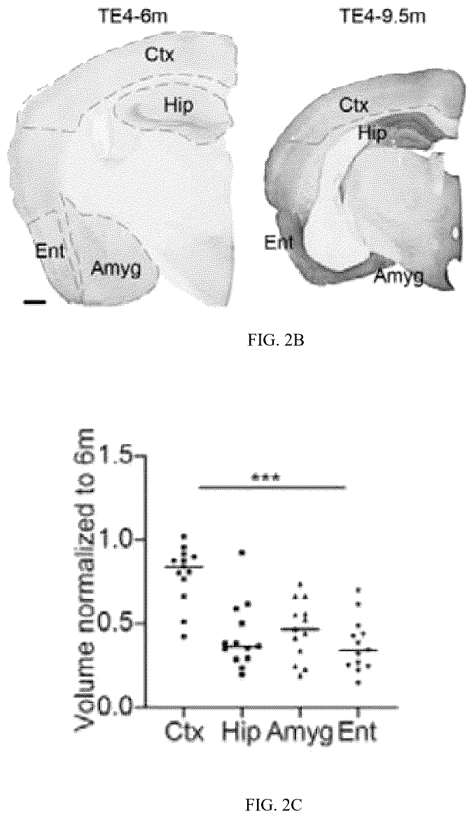

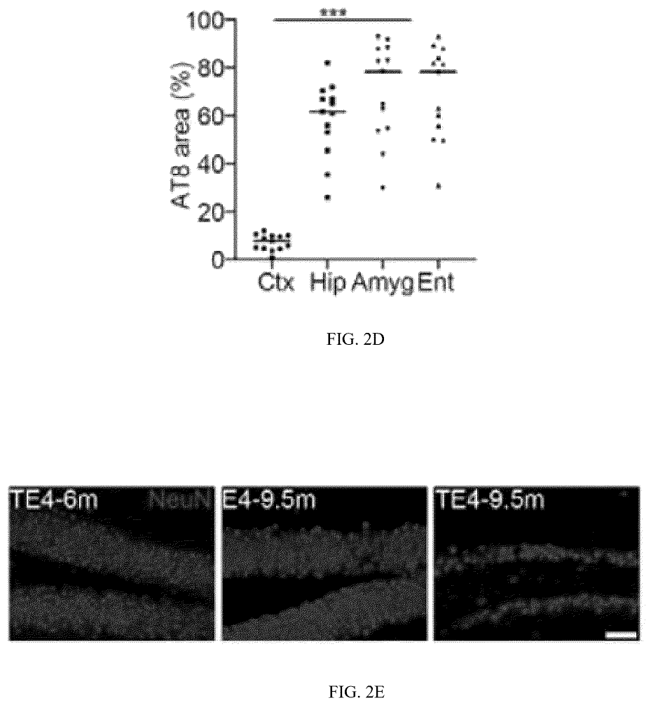

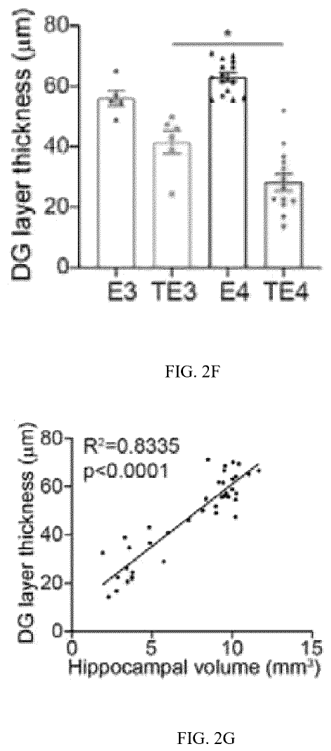

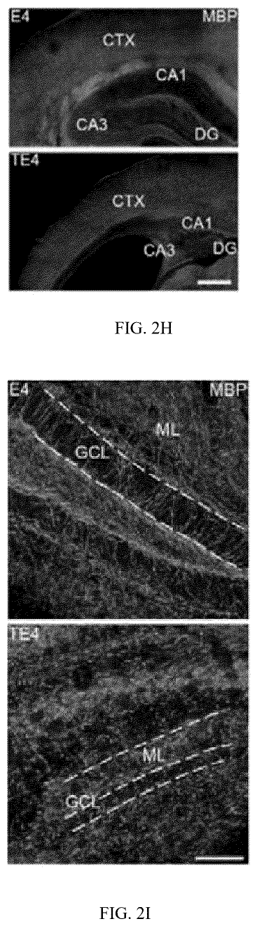

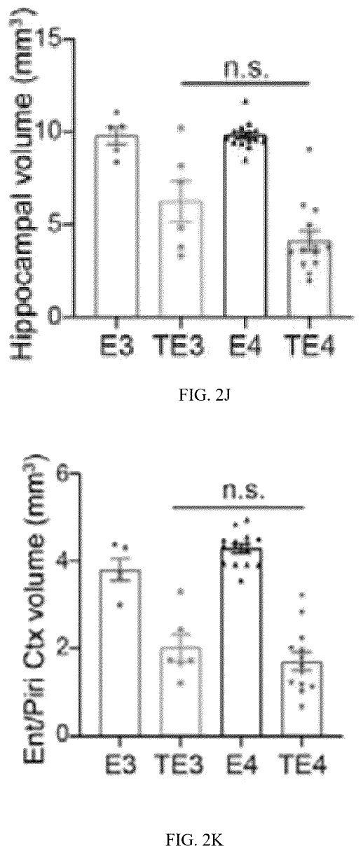

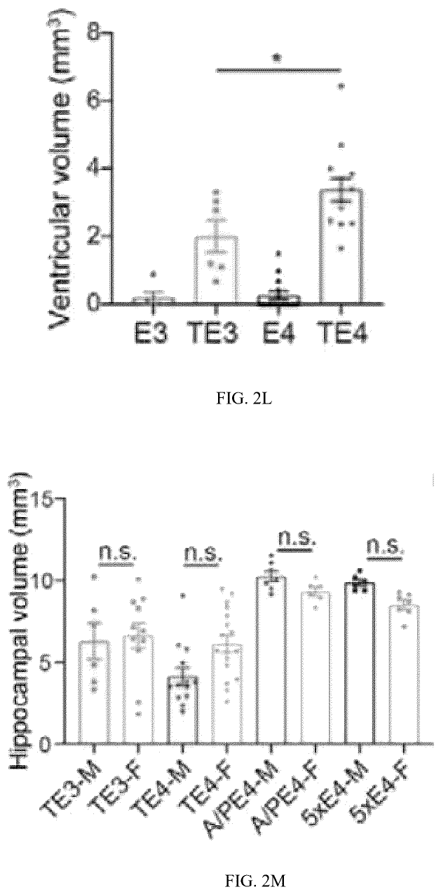

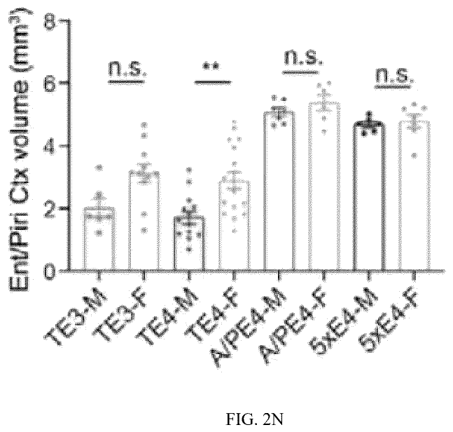

To explore the disease microenvironment in the presence of amyloid or tau deposition, the immunological milieus were systematically compared in the brains of Aβ depositing mice APP/PS1-21 (A/PE4) and 5×FADE4 (5×E4), and tauopathy (TE4) mice that express human APOE4 (E4). The pathologies in these models mirror amyloid deposition and tau aggregation with neurodegeneration, respectively. Significant brain regional atrophy was observed by 9.5 months but not at 6 months of age in TE4 mice (FIG. 1A). In addition, brain atrophy was not present in A/PE4 nor 5×E4 mice by 9.5 months of age despite massive Aβ deposition in the brain (FIG. 1A and FIG. 2A). The atrophy in the TE4 mice at 9.5 months primarily occurred in regions that developed the most tauopathy (i.e. the hippocampus, piriform/entorhinal cortex, and amygdala) and was accompanied by significant lateral ventricular enlargement (FIGS. 1A-D and FIGS. 2B-D). The granule cell layer in the dentate gyrus (DG) as assessed by Neun staining was noticeably decreased in TE4 mice, and the thickness correlated highly with hippocampal volume (FIG. 2E-G). Consistent with the neuronal loss, myelin basic protein (MBP)-positive staining, which is present around intact axons, was altered in TE4 mice at 9.5 months (FIG. 2H, 2I). Both TE4 and TE3 (expressing human APOE3) mice developed prominent brain atrophy with somewhat greater atrophy in the TE4 mice (FIG. 2J-L). Additionally, male mice tended to have higher levels of brain atrophy than that of females (FIG. 2M-O). For further exploration of mechanisms of brain atrophy/neurodegeneration, male mice were focused on, for the remainder of the experiments.

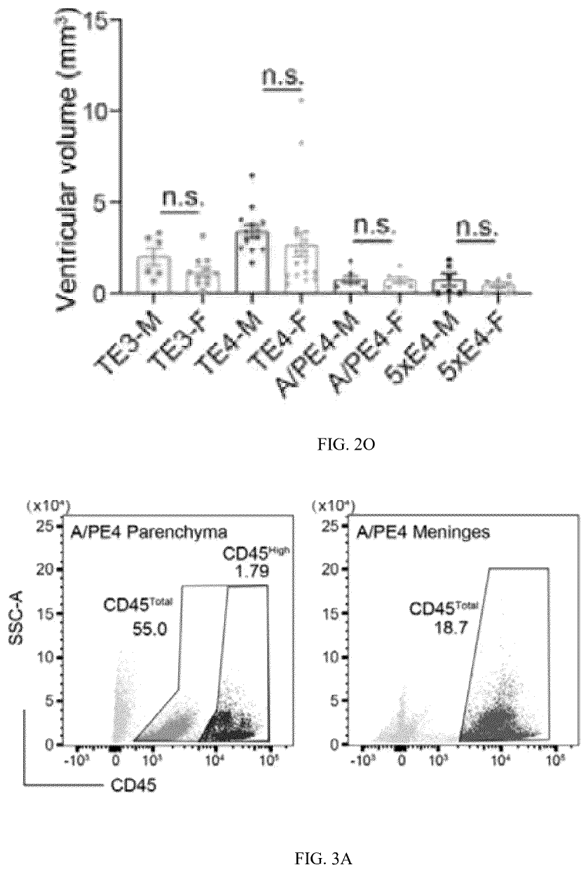

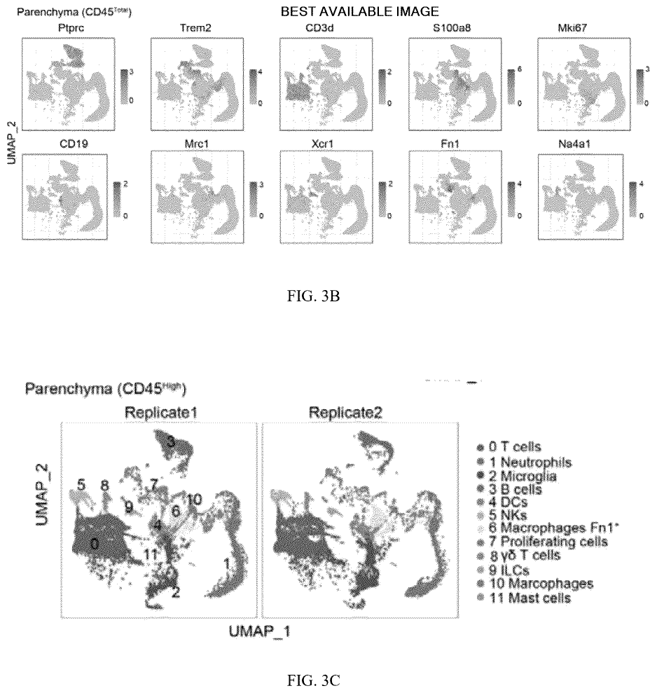

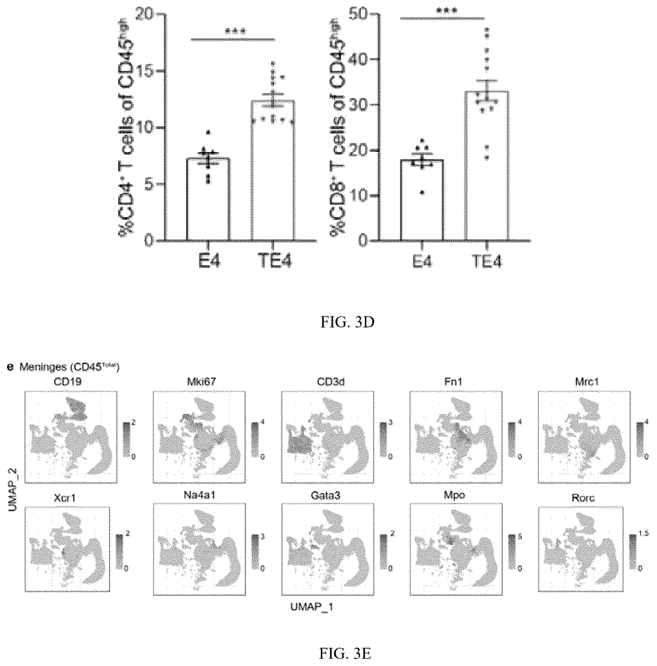

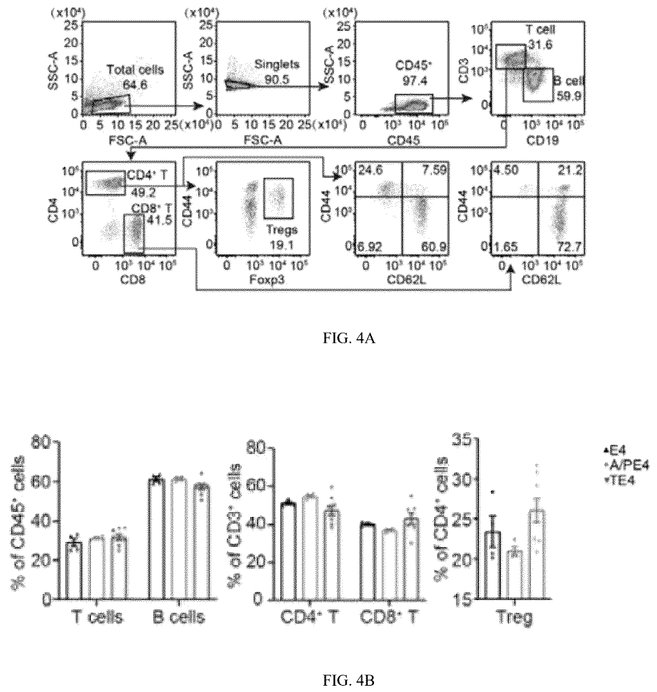

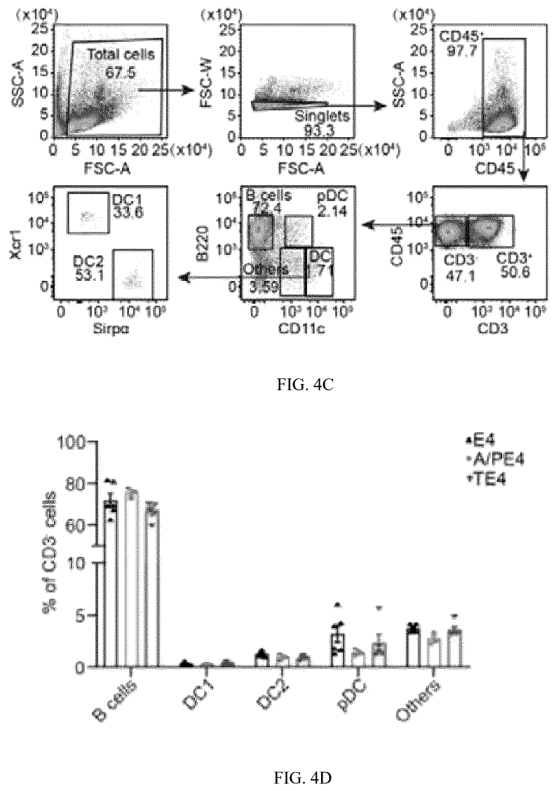

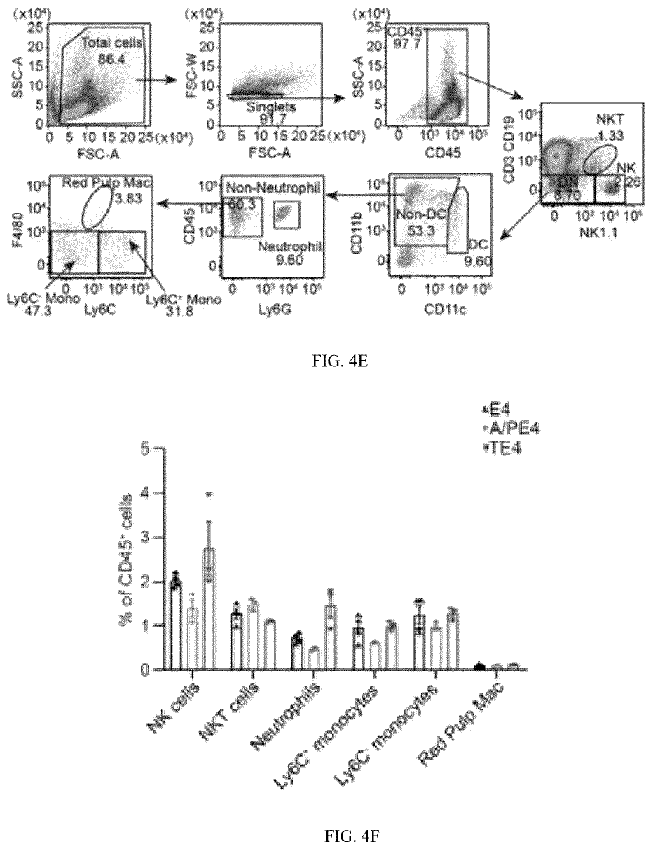

To map a full picture of the innate and adaptive immune responses in the presence of Aβ or tau pathology, a cellular and molecular atlas of the meningeal and parenchymal immune cell niche via single cell immune sequencing was generated, on sorted total CD45-positive cells (CD45^{Total}) from meninges and CD45^{Total} and CD45-high cells (CD45^{high}) cells from the brain parenchyma in APOE4 knockin mice (E4), A/PE4 and TE4 male mice at 9.5 months with matched genetic background (FIG. 1E, FIG. 3A and Table 2). Unsupervised clustering identified 12 robust cell types of CD45^{Total} in the parenchyma of E4, A/PE4 and TE4 mice, i.e. microglia, T cells, Neutrophils, proliferating cells, B cells, DCs, NK cells, macrophages, γδ T cells, innate lymphocyte cells (ILCs) and mast cells (FIG. 1F and FIG. 3B). Surprisingly, the percentage of the T cell population of the total immune cells was strongly increased in the TE4 mice as compared with A/PE4 and E4 mice (FIG. 1G). In fluorescence activated cell sorting (FACS) analysis, the proportion of CD45^{high} cells, which mainly represents the adaptive immune cell populations and innate immune cells such as DCs and macrophages, were enriched in the brain parenchyma of 9.5 month TE4 mice (FIG. 1E, FIG. 3A, and FIG. 3C). Consistent with scRNAseq data, we observed a significant increase in CD4 and CD8 T cells in TE4 vs. E4 mice, and CD8 cells were the more abundant population (FIG. 3D). The meninges are a triple layer structure enveloping the brain and are an immune blood-brain interface. During aging and neurodegenerative diseases, dysfunctional lymphatic vessels lead to impaired drainage, which appears to result in dysregulated immune cell trafficking. Distinct cell types were observed in CD45^{Total} populations (FIG. 1H, FIG. 1I, FIG. 3E, and Table 3). In addition, the peripheral immune cell composition as assessed in the spleen was not significantly changed in TE4 mice as compared to E4 and A/PE4 mice (FIG. 4A-F). In summary, these results reveal comprehensive and distinct innate and adaptive immune niches present in the parenchyma and an increased proportion of T cells in the presence of tauopathy and neurodegeneration.

TABLE 2
Single cell immune seq sample information

| | | | | | Brain | Total | Microglia | Median | | Median | Number |
|---|---|---|---|---|---|---|---|---|---|---|---|
| | Age | | | Genetic | regions for | cell | cell | genes per | T cell | genes per | of |
| ID | (months) | Sex | Genetics | background | sequencing | number | number | microglia | number | T cell | Mice |
| 1 | 9.5 | M | E4- | BL6 | CD45^{Total} | 22248 | 20547 | 2091 | 489 | 2044 | 10 |
| | | | Replicate1 | | Cortex | | | | | | |
| | | | | | and | | | | | | |
| | | | | | Hippocampus | | | | | | |
| 2 | 9.5 | M | E4- | BL6 | CD45^{Total} | 4640 | 4107 | 1466 | 198 | 1762 | 5 |
| | | | Replicate2 | | Cortex | | | | | | |
| | | | | | and | | | | | | |
| | | | | | Hippocampus | | | | | | |
| 3 | 9.5 | M | A/PE4 | BL6 | CD45^{Total} | 9869 | 9216 | 2019 | 263 | 3044 | 5 |
| | | | | | Cortex | | | | | | |
| | | | | | and | | | | | | |
| | | | | | Hippocampus | | | | | | |
| 4 | 9.5 | M | TE4- | BL6 | CD45^{Total} | 8570 | 7210 | 2913 | 745 | 2111 | 5 |
| | | | Replicate1 | | Cortex | | | | | | |
| | | | | | and | | | | | | |
| | | | | | Hippocampus | | | | | | |
| 5 | 9.5 | M | TE4- | BL6 | CD45^{Total} | 6305 | 4915 | 1470 | 700 | 1782 | 5 |
| | | | Replicate2 | | Cortex | | | | | | |
| | | | | | and | | | | | | |
| | | | | | Hippocampus | | | | | | |
| 6 | 9.5 | M | E4- | BL6 | CD45high | 9489 | 501 | 2158 | 3396 | 1897 | 10 |
| | | | Replicate1 | | Cortex | | | | | | |
| | | | | | and | | | | | | |
| | | | | | Hippocampus | | | | | | |
| 7 | 9.5 | M | E4- | BL6 | CD45^{high} | 6019 | 544 | 2029 | 2251 | 1498 | 5 |
| | | | Replicate2 | | Cortex | | | | | | |
| | | | | | and | | | | | | |
| | | | | | Hippocampus | | | | | | |
| 8 | 9.5 | M | A/PE4 | BL6 | CD45^{high} | 7161 | 1352 | 2822 | 2300 | 1748 | 5 |
| | | | | | Cortex | | | | | | |
| | | | | | and | | | | | | |
| | | | | | Hippocampus | | | | | | |
| 9 | 9.5 | M | TE4- | BL6 | CD45^{high} | 6741 | 292 | 2060 | 4587 | 2352 | 5 |
| | | | Replciate1 | | Cortex | | | | | | |
| | | | | | and | | | | | | |
| | | | | | Hippocampus | | | | | | |
| 10 | 9.5 | M | TE4- | BL6 | CD45^{high} | 11364 | 1765 | 2322 | 5602 | 1740 | 5 |
| | | | Replicate2 | | Cortex | | | | | | |
| | | | | | and | | | | | | |
| | | | | | Hippocampus | | | | | | |
| 11 | 9.5 | M | E4- | BL6 | CD45^{Total} | 27042 | 950 | 1718 | 1600 | 1708 | 10 |
| | | | Replicate1 | | Meninges | | | | | | |
| 13 | 9.5 | M | A/PE4 | BL6 | CD45^{Total} | 11197 | 593 | 1786 | 468 | 1517 | 5 |
| | | | | | Meninges | | | | | | |
| 14 | 9.5 | M | TE4- | BL6 | CD45^{Total} | 14314 | 80 | 2886 | 1497 | 1953 | 5 |
| | | | Replicate1 | | Meninges | | | | | | |
| 15 | 9.5 | M | TE4- | BL6 | CD45^{Total} | 10741 | 653 | 1658 | 1053 | 1380 | 5 |
| | | | Replicate2 | | Meninges | | | | | | |
| 16 | 9.5 | M | TE4- | BL6 | CD45^{Total} | \|12572 | 11310 | 2068 | 335 | 1779 | 5 |
| | | | Ctrl | | Cortex and | | | | | | |
| | | | (lgG) | | Hippocampus | | | | | | |
| 17 | 9.5 | M | \|TE4-aT | BL6 | CD45^{Total} | 15080 | 14009 | 1974 | 74 | 1605 | 5 |
| | | | | | Cortex | | | | | | |
| | | | | | and | | | | | | |
| | | | | | Hippocampus | | | | | | |
| 12 | 9.5 | M | E4- | BL6 | CD45^{Total} | 13554 | 1358 | 2030 | 1279 | 1298 | 5 |
| | | | Replicate2 | | Meninges | | | | | | |

TABLE 3
CD45^{Total} immune cell number per cluster in meninges

| | | | Total | Cell | Per- |
|---|---|---|---|---|---|
| | Geno- | Re- | cell | number/ | centage |
| Cluster | type | plica | number | Cluster | (%) |
| 0 Neutrophils | E4 | Rep1 | 27042 | 10165 | 37.6 |
| 0 Neutrophils | E4 | Rep2 | 13554 | 5061 | 37.3 |
| 0 Neutrophils | A/PE4 | Rep1 | 11197 | 3963 | 35.4 |
| 0 Neutrophils | TE4 | Rep1 | 14314 | 5923 | 41.4 |
| 0 Neutrophils | TE4 | Rep2 | 10741 | 4059 | 37.8 |
| 1 B cells | E4 | Rep1 | 27042 | 6590 | 24.4 |
| 1 B cells | E4 | Rep2 | 13554 | 1632 | 12.0 |
| 1 B cells | A/PE4 | Rep1 | 11197 | 3237 | 28.9 |
| 1 B cells | TE4 | Rep1 | 14314 | 2394 | 16.7 |
| 1 B cells | TE4 | Rep2 | 10741 | 1977 | 18.4 |
| 2 Proliferating cells | E4 | Rep1 | 27042 | 3392 | 12.5 |
| 2 Proliferating cells | E4 | Rep2 | 13554 | 1327 | 9.8 |
| 2 Proliferating cells | A/PE4 | Rep1 | 11197 | 1336 | 11.9 |
| 2 Proliferating cells | TE4 | Rep1 | 14314 | 1337 | 9.3 |
| 2 Proliferating cells | TE4 | Rep2 | 10741 | 1252 | 11.7 |
| 3 T cells | E4 | Rep1 | 27042 | 1600 | 5.9 |
| 3 T cells | E4 | Rep2 | 13554 | 1279 | 9.4 |
| 3 T cells | A/PE4 | Rep1 | 11197 | 468 | 4.2 |
| 3 T cells | TE4 | Rep1 | 14314 | 1497 | 10.5 |
| 3 T cells | TE4 | Rep2 | 10741 | 1053 | 9.8 |
| 4 Macrophages Fn1⁺ | E4 | Rep1 | 27042 | 1701 | 6.3 |
| 4 Macrophages Fn1⁺ | E4 | Rep2 | 13554 | 916 | 6.8 |
| 4 Macrophages Fn1⁺ | A/PE4 | Rep1 | 11197 | 635 | 5.7 |
| 4 Macrophages Fn1⁺ | TE4 | Rep1 | 14314 | 959 | 6.7 |
| 4 Macrophages Fn1⁺ | TE4 | Rep2 | 10741 | 570 | 5.3 |
| 5 DCs | E4 | Rep1 | 27042 | 1308 | 4.8 |
| 5 DCs | E4 | Rep2 | 13554 | 997 | 7.4 |
| 5 DCs | A/PE4 | Rep1 | 11197 | 574 | 5.1 |
| 5 DCs | TE4 | Rep1 | 14314 | 1132 | 7.9 |
| 5 DCs | TE4 | Rep2 | 10741 | 518 | 4.8 |
| 6 Microglia | E4 | Rep1 | 27042 | 950 | 3.5 |
| 6 Microglia | E4 | Rep2 | 13554 | 1358 | 10.0 |
| 6 Microglia | A/PE4 | Rep1 | 11197 | 593 | 5.3 |
| 6 Microglia | TE4 | Rep1 | 14314 | 80 | 0.6 |
| 6 Microglia | TE4 | Rep2 | 10741 | 653 | 6.1 |
| 7 NKs | E4 | Rep1 | 27042 | 427 | 1.6 |
| 7 NKs | E4 | Rep2 | 13554 | 307 | 2.3 |
| 7 NKs | A/PE4 | Rep1 | 11197 | 122 | 1.1 |
| 7 NKs | TE4 | Rep1 | 14314 | 385 | 2.7 |
| 7 NKs | TE4 | Rep2 | 10741 | 197 | 1.8 |
| 8 Macrophages | E4 | Rep1 | 27042 | 489 | 1.8 |
| 8 Macrophages | E4 | Rep2 | 13554 | 299 | 2.2 |
| 8 Macrophages | A/PE4 | Rep1 | 11197 | 107 | 1.0 |
| 8 Macrophages | TE4 | Rep1 | 14314 | 293 | 2.0 |
| 8 Macrophages | TE4 | Rep2 | 10741 | 232 | 2.2 |
| 9 ILCs | E4 | Rep1 | 27042 | 240 | 0.9 |
| 9 ILCs | E4 | Rep2 | 13554 | 176 | 1.3 |
| 9 ILCs | A/PE4 | Rep1 | 11197 | 96 | 0.9 |
| 9 ILCs | TE4 | Rep1 | 14314 | 159 | 1.1 |
| 9 ILCs | TE4 | Rep2 | 10741 | 104 | 1.0 |
| 10 Mast cells | E4 | Rep1 | 27042 | 134 | 0.5 |
| 10 Mast cells | E4 | Rep2 | 13554 | 95 | 0.7 |
| 10 Mast cells | A/PE4 | Rep1 | 11197 | 48 | 0.4 |
| 10 Mast cells | TE4 | Rep1 | 14314 | 82 | 0.6 |
| 10 Mast cells | TE4 | Rep2 | 10741 | 77 | 0.7 |
| 11 yδ T cells | E4 | Rep1 | 27042 | 46 | 0.2 |
| 11 yδ T cells | E4 | Rep2 | 13554 | 107 | 0.8 |
| 11 yδ T cells | A/PE4 | Rep1 | 11197 | 18 | 0.2 |
| 11 yδ T cells | TE4 | Rep1 | 14314 | 73 | 0.5 |
| 11 yδ T cells | TE4 | Rep2 | 10741 | 49 | 0.5 |

### Example 3: T Cell Increase in Brain Parenchyma with Tau Pathology

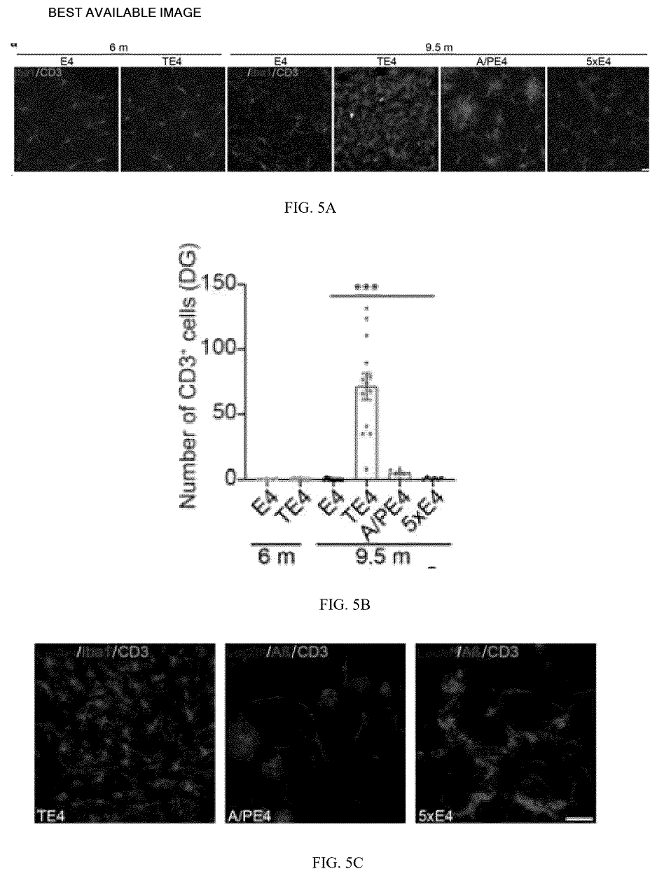

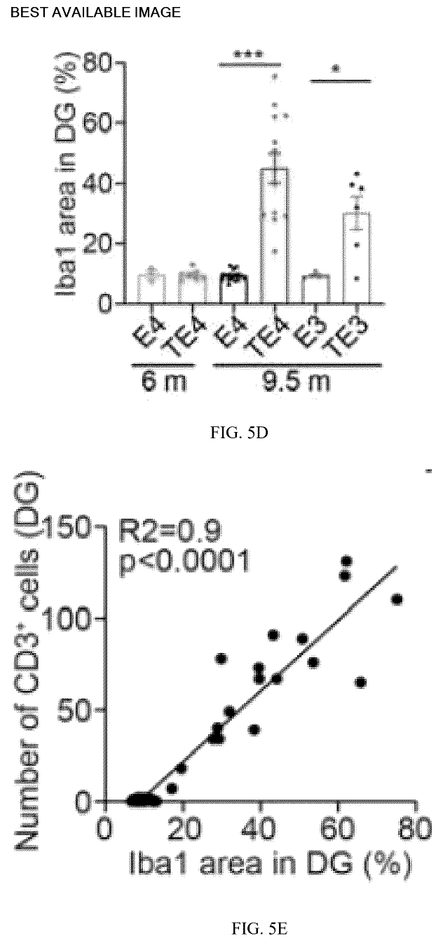

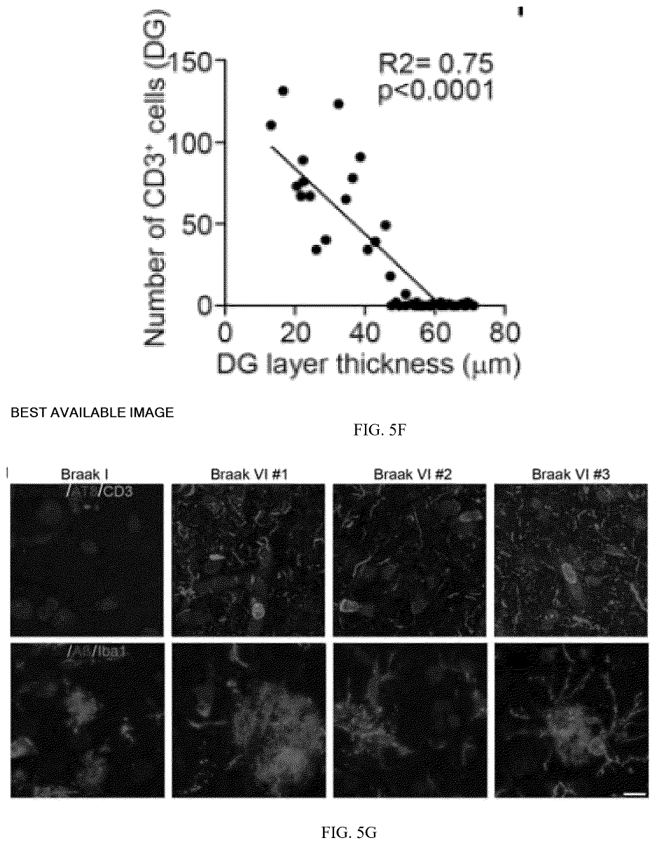

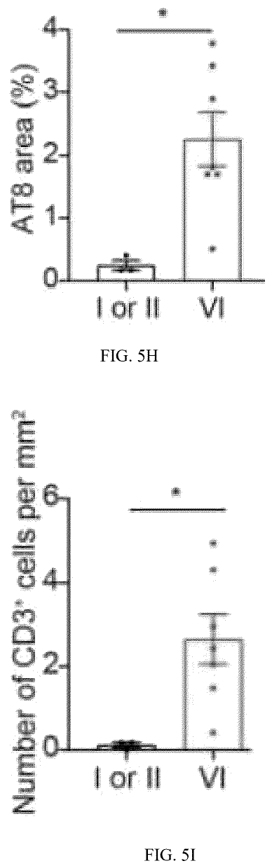

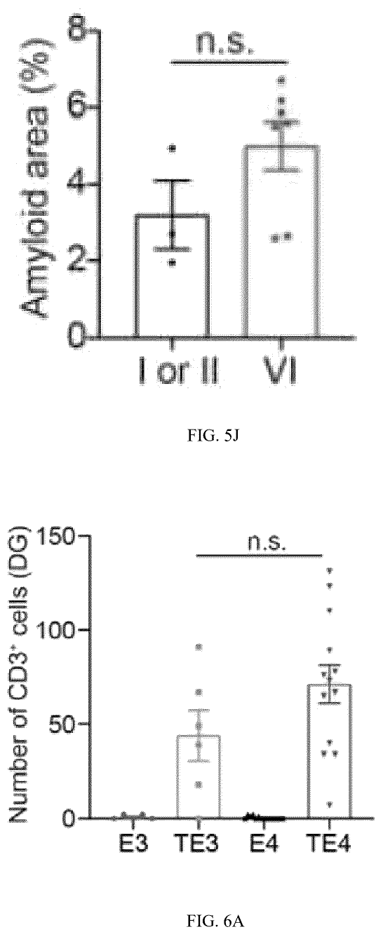

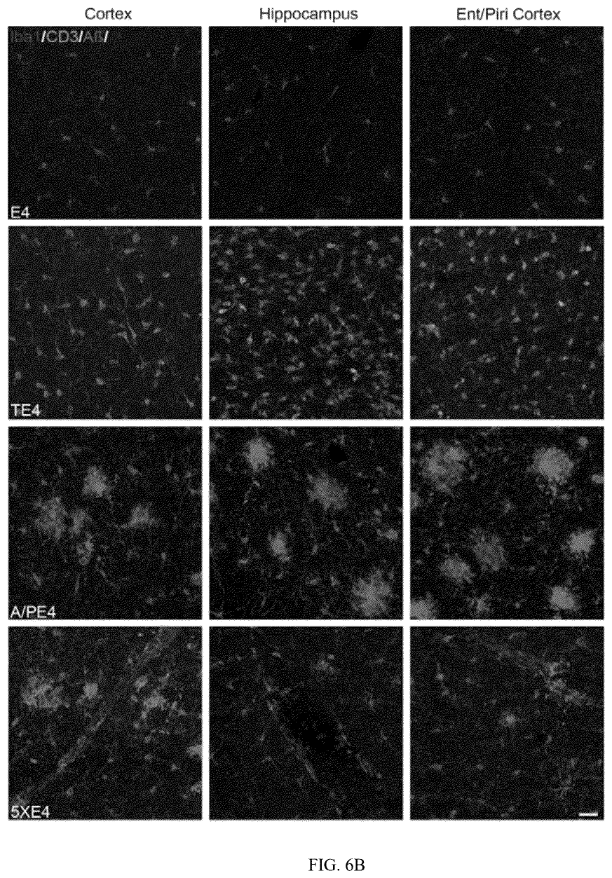

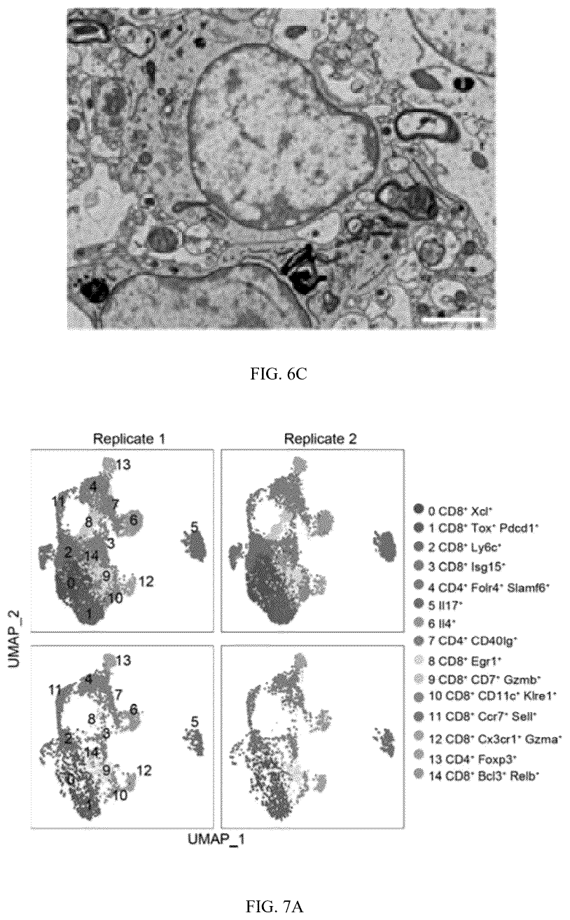

To further investigate the apparent expansion of T cells observed in single cell immune RNA sequencing data, immunohistochemical analyses was performed in the parenchyma from TE4, A/PE4 and 5×E4 mice using antibodies to cluster of differentiation 3 (CD3) and ionized calcium binding adaptor molecule 1 (Iba1), pan markers for T cells and microglia, respectively. It was found that T cells were significantly elevated in 9.5 month TE4 mice, but not in 9.5 month E4 controls or in 6-month-old TE4 mice (FIG. 5A, FIG. 5B, FIG. 6A, and FIG. 6B). Increased T cells were also found in TE3 mice (FIG. 6A) and Tau mice expressing mouse ApoE, suggesting a linkage between T cells and tau-mediated neurodegeneration rather than requiring a specific ApoE isoform. Interestingly, T cells were not obviously increased in amyloid-depositing A/PE4 and 5×E4 mice at 9.5 months of age or even at 19 months of age compared with TE4 mice (FIG. 5A-C). Further, CD3 staining was primarily present in hippocampus and piriform/entorhinal cortex, which are regions with hyperphosphorylated tau accumulation and neuronal loss, indicating a possible detrimental role for T cells in tau-dependent neurodegeneration (FIG. 6B). In accordance with the increase of infiltrated T cells, microglia were also significantly elevated in 9.5 month TE4 mice in regions with brain atrophy (FIG. 5A, FIG. 5D). The number of T cells showed a positive correlation with the number of microglia (FIG. 5E) and negatively correlated with the granule cell layer thickness in DG (FIG. 5F). To assess whether the T cells were localized in the brain parenchyma as opposed to within the vasculature, brain vessels were co-stained with retro-orbital Lectin-dye injection and CD3, and it was noted that CD3⁺ cells were not present in the lumen of blood vessels (FIG. 5C). Furthermore, transmission electron microscopy (TEM) also revealed that T cells were in the parenchyma adjacent to other cells in the brain (FIG. 6C). To determine whether a similar tau pathology-associated increase of T cells was present in the parenchyma in human AD, immunohistochemical analyses were performed in brain samples of AD patients (superior frontal gyrus) with low (I-II) and high (VI) Braak stages (FIG. 5G and Table 1). In line with the amount of phosphorylated tau (p-Tau) pathology, CD3⁺ T cells were strongly elevated in the superior frontal gyrus from Braak stage VI vs. the Braak stage I or II cases (FIG. 5G, FIG. 5H, FIG. 5I). By contrast, in these samples, overall Aβ deposition was similar in brain tissues with both low and high Braak stages (FIG. 5G, FIG. 2J). To summarize, the data demonstrated presence of increased parenchymal T cells in brain regions with tauopathy but not in the presence of only amyloid deposition, in both humans and mice.

### Example 4: T Cells Shift from Activated to Exhausted States with Tau Pathology

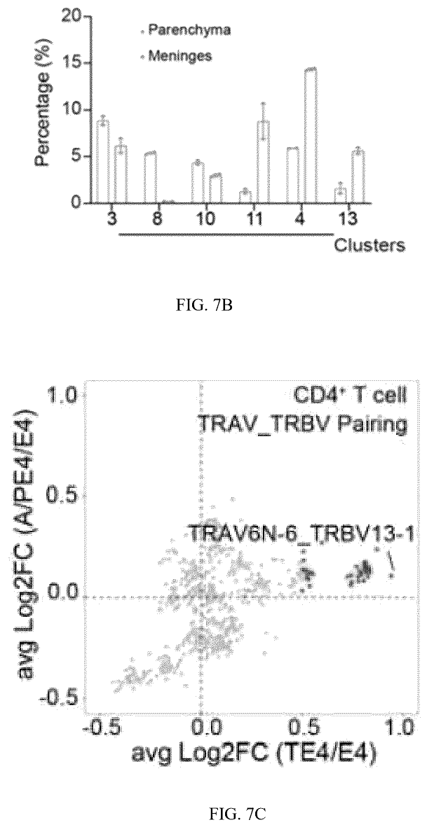

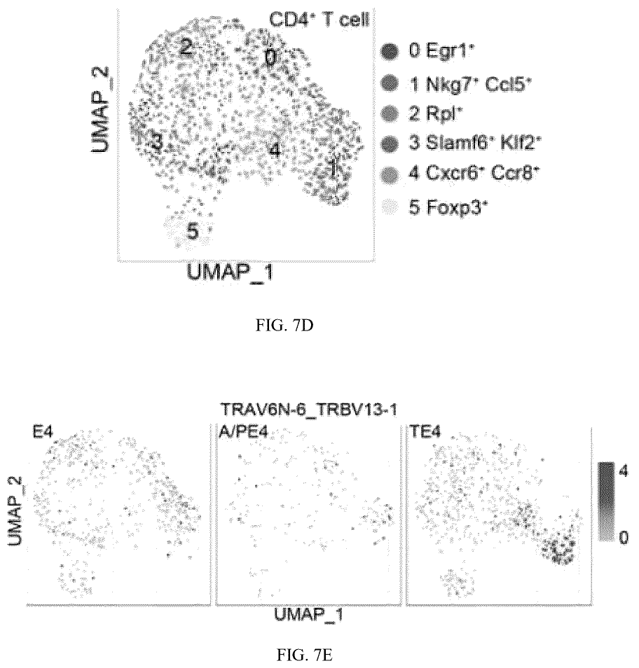

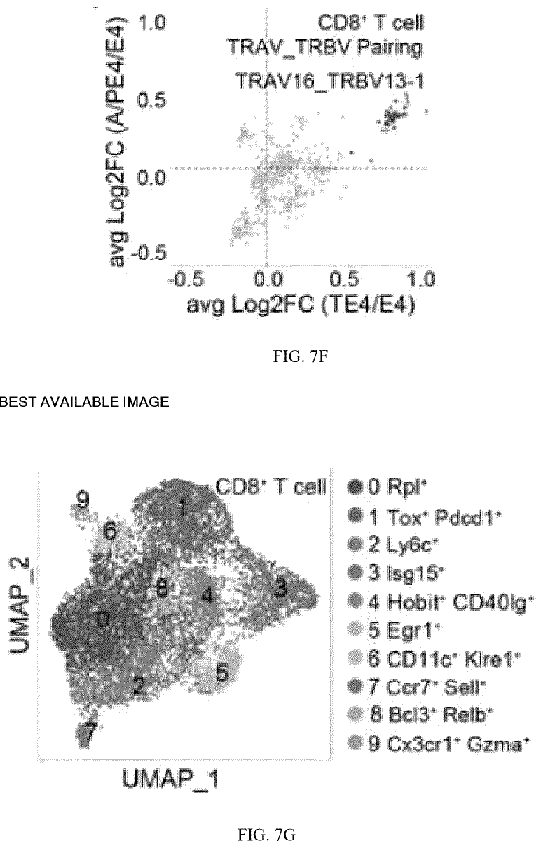

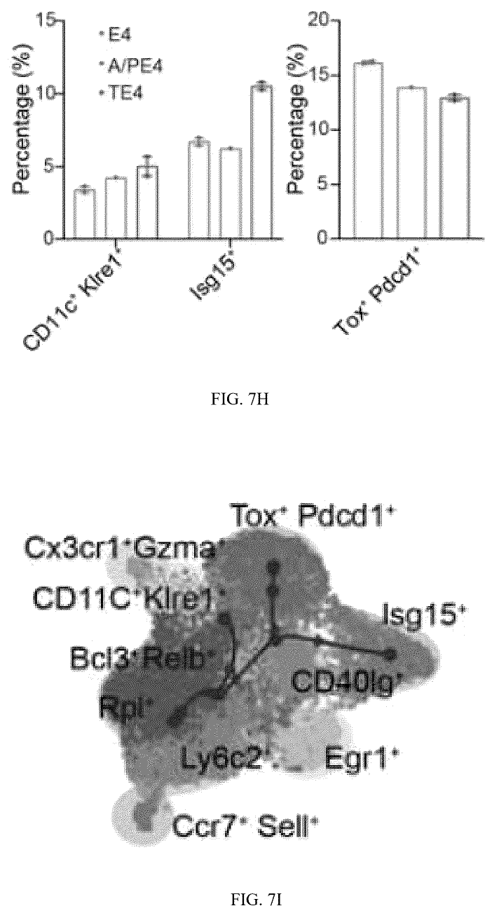

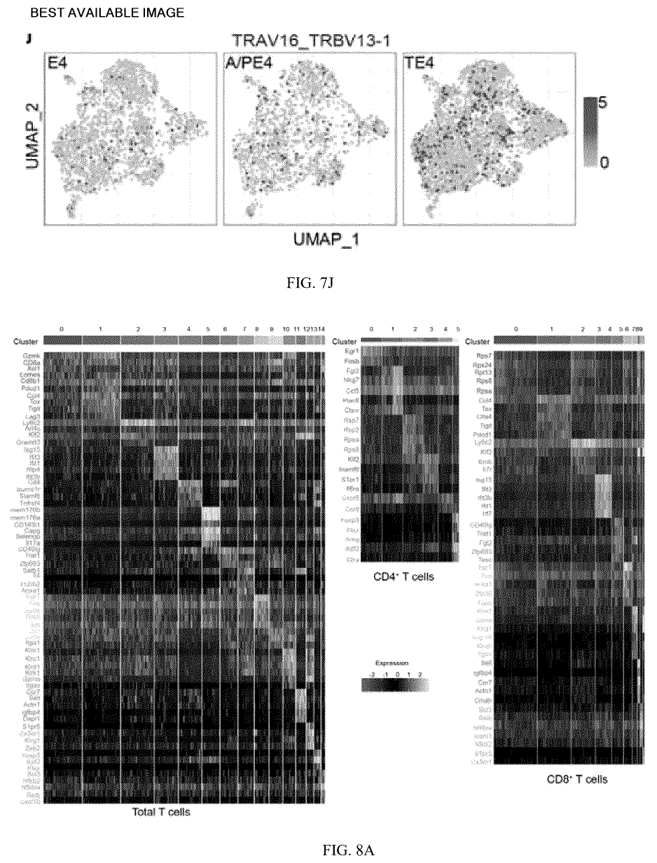

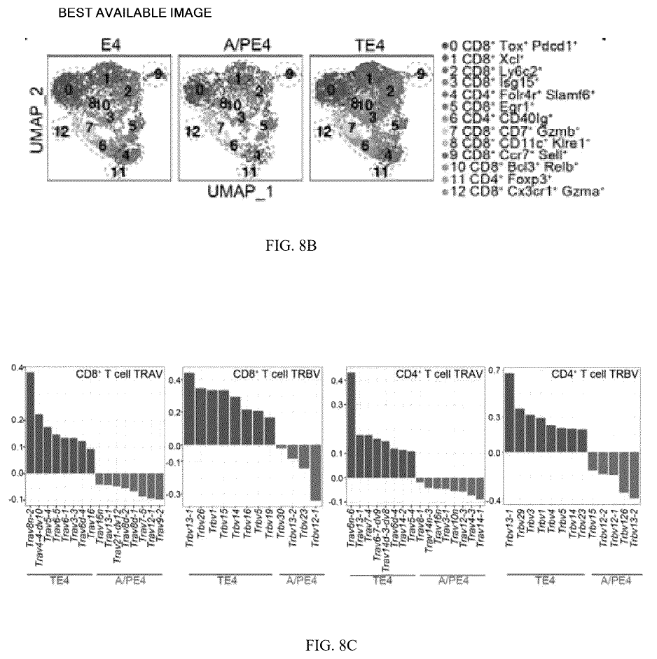

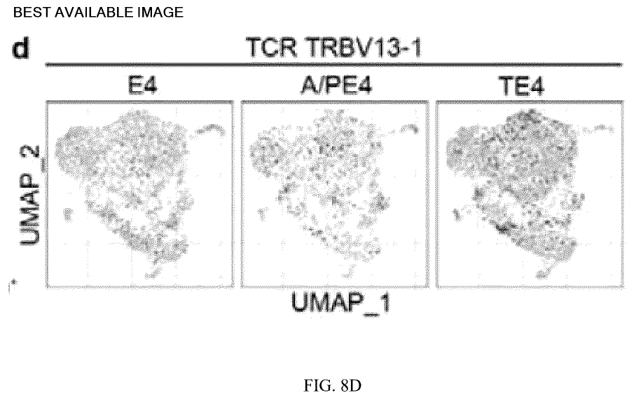

To depict the cellular and molecular signatures of the T cells in the presence of Aβ or tau pathology, T cell populations were assessed from single cell immune RNA-seq data from CD45^{high} cells in the parenchyma and CD45^{Total} cells in the meninges in E4, A/PE4 and TE4 mice. T cells were categorized into 15 subgroups across all samples based on expression of featured genes (FIG. 7A, FIG. 8A and Table 4). Cell population analysis revealed population differences between parenchyma and meninges. Naïve CD8⁺ T cells (subgroup 11), Folr4⁺CD4⁺ T cells (subgroup 4) and Regulatory T cells (subgroup 13) were highly enriched in meninges, but effector CD8⁺ T cells (subgroups 3, 8, 10) were preferentially enriched in brain parenchyma (FIG. 7B and Table 5). These results suggest that brain-border and brain-resident T cells are functionally different in accordance with their immune niche. Interaction between the T cell receptor (TCR) and antigens presented by the major histocompatibility complex (MHC) is critical to adaptive immunity. When T cells recognize cognate antigen, they clonally expand. Single cell TCR sequencing (scTCR-seq) on T cells was performed, which showed unique T cell clonal enrichment in the parenchyma with tauopathy and neurodegeneration (FIG. 8B-D). Further, TCR repertoires were evaluated among CD4⁺ T cell subsets. An increased clonality in CD4⁺ T cells in TE4 mice was observed that was concentrated within the activated CD4⁺ T cells (Nkg7⁺Ccl5⁺ and Cxcr6⁺Ccr8⁺CD4⁺ T cells) (FIGS. 7C-E and Table 6). Similar to what was found in CD4⁺ T cells, paired TCRα/TCRβ repertoire analysis revealed TCR clonal expansion in CD8⁺ T cells in TE4 mice (FIG. 7F). Unsupervised clustering identified 10 robust cell types in CD8⁺ T cells (FIG. 7G, FIG. 8, Table 4 and Table 7). Activated CD8⁺ T cells (CD11c⁺Klre1⁺ and Isg15⁺CD8⁺ T cells) were more abundant in TE4 mice, while the fraction of Tox⁺Pdcd1⁺CD8⁺ exhausted T cells were slightly decreased, suggesting a potential role of activated CD8⁺ T cells in mediating neuronal loss in tauopathy (FIG. 7H). Pseudotime analysis of CD8⁺ T cells found a range of T cell states indicative of a dynamic shift from activated to exhausted states (FIG. 7I). An increased clonality was also observed in activated and exhausted CD8⁺ T cells in TE4 mice (FIG. 7J). Together, the data illustrated that T cells in the brain parenchyma dynamically shift from activated to exhausted states with unique TCR clonal expansion in both CD4⁺ and CD8⁺ populations in the brain in a mouse model of tauopathy.

TABLE 4
Immune cell number of each cluster in
parenchyma and meninges CD45^{Total} immune
cell number per cluster in parenchyma

| | | | Total | Cell | Per- |
|---|---|---|---|---|---|
| | Geno- | Re- | cell | number/ | centage |
| Cluster | type | plica | number | Cluster | (%) |
| 0 Microglia | E4 | Rep1 | 22248 | 20547 | 92.4 |
| 0 Microglia | E4 | Rep2 | 4640 | 4107 | 88.5 |
| 0 Microglia | A/PE4 | Rep1 | 9869 | 9216 | 93.4 |
| 0 Microglia | TE4 | Rep1 | 8570 | 7210 | 84.1 |
| 0 Microglia | TE4 | Rep2 | 6305 | 4915 | 78.0 |
| 1 T cells | E4 | Rep1 | 22248 | 489 | 2.2 |
| 1 T cells | E4 | Rep2 | 4640 | 198 | 4.3 |
| 1 T cells | A/PE4 | Rep1 | 9869 | 263 | 2.7 |
| 1 T cells | TE4 | Rep1 | 8570 | 745 | 8.7 |
| 1 T cells | TE4 | Rep2 | 6305 | 700 | 11.1 |
| 2 Proliferating | E4 | Rep1 | 22248 | 226 | 1.0 |
| cells | | | | | |
| 2 Proliferating | E4 | Rep2 | 4640 | 74 | 1.6 |
| cells | | | | | |
| 2 Proliferating | A/PE4 | Rep1 | 9869 | 145 | 1.5 |
| cells | | | | | |
| 2 Proliferating | TE4 | Rep1 | 8570 | 114 | 1.3 |
| cells | | | | | |
| 2 Proliferating | TE4 | Rep2 | 6305 | 244 | 3.9 |
| cells | | | | | |
| 3 Neutrophils | E4 | Rep1 | 22248 | 349 | 1.6 |
| 3 Neutrophils | E4 | Rep2 | 4640 | 54 | 1.2 |
| 3 Neutrophils | A/PE4 | Rep1 | 9869 | 101 | 1.0 |
| 3 Neutrophils | TE4 | Rep1 | 8570 | 118 | 1.4 |
| 3 Neutrophils | TE4 | Rep2 | 6305 | 149 | 2.4 |
| 4 B cells | E4 | Rep1 | 22248 | 207 | 0.9 |
| 4 B cells | E4 | Rep2 | 4640 | 84 | 1.8 |
| 4 B cells | A/PE4 | Rep1 | 9869 | 32 | 0.3 |
| 4 B cells | TE4 | Rep1 | 8570 | 64 | 0.7 |
| 4 B cells | TE4 | Rep2 | 6305 | 57 | 0.9 |
| 5 DCs | E4 | Rep1 | 22248 | 126 | 0.6 |
| 5 DCs | E4 | Rep2 | 4640 | 37 | 0.8 |
| 5 DCs | A/PE4 | Rep1 | 9869 | 28 | 0.3 |
| 5 DCs | TE4 | Rep1 | 8570 | 93 | 1.1 |
| 5 DCs | TE4 | Rep2 | 6305 | 116 | 1.8 |
| 6 NKs | E4 | Rep1 | 22248 | 97 | 0.4 |
| 6 NKs | E4 | Rep2 | 4640 | 21 | 0.5 |
| 6 NKs | A/PE4 | Rep1 | 9869 | 14 | 0.1 |
| 6 NKs | TE4 | Rep1 | 8570 | 76 | 0.9 |
| 6 NKs | TE4 | Rep2 | 6305 | 55 | 0.9 |
| 7 Macrophages | E4 | Rep1 | 22248 | 92 | 0.4 |
| Fn1+ | | | | | |
| 7 Macrophages | E4 | Rep2 | 4640 | 19 | 0.4 |
| Fn1⁺ | | | | | |
| 7 Macrophages | A/PE4 | Rep1 | 9869 | 14 | 0.1 |
| Fn1⁺ | | | | | |
| 7 Macrophages | TE4 | Rep1 | 8570 | 37 | 0.4 |
| Fn1⁺ | | | | | |
| 7 Macrophages | TE4 | Rep2 | 6305 | 20 | 0.3 |
| Fn1⁺ | | | | | |
| 8 yδ T cells | E4 | Rep1 | 22248 | 19 | 0.1 |
| 8 yδ T cells | E4 | Rep2 | 4640 | 18 | 0.4 |
| 8 yδ T cells | A/PE4 | Rep1 | 9869 | 26 | 0.3 |
| 8 yδ T cells | TE4 | Rep1 | 8570 | 54 | 0.6 |
| 8 γδ T cells | TE4 | Rep2 | 6305 | 34 | 0.5 |
| 9 Macrophages | E4 | Rep1 | 22248 | 57 | 0.3 |
| 9 Macrophages | E4 | Rep2 | 4640 | 19 | 0.4 |
| 9 Macrophages | A/PE4 | Rep1 | 9869 | 22 | 0.2 |
| 9 Macrophages | TE4 | Rep1 | 8570 | 31 | 0.4 |
| 9 Macrophages | TE4 | Rep2 | 6305 | 5 | 0.1 |
| 10 ILCs | E4 | Rep1 | 22248 | 33 | 0.1 |
| 10 ILCs | E4 | Rep2 | 4640 | 4 | 0.1 |
| 10 ILCs | A/PE4 | Rep1 | 9869 | 7 | 0.1 |
| 10 ILCs | TE4 | Rep1 | 8570 | 24 | 0.3 |
| 10 ILCs | TE4 | Rep2 | 6305 | 9 | 0.1 |
| 11 Mast cells | E4 | Rep1 | 22248 | 6 | 0.0 |
| 11 Mast cells | E4 | Rep2 | 4640 | 5 | 0.1 |
| 11 Mast cells | A/PE4 | Rep1 | 9869 | 1 | 0.0 |
| 11 Mast cells | TE4 | Rep1 | 8570 | 4 | 0.0 |
| 11 Mast cells | TE4 | Rep2 | 6305 | 1 | 0.0 |

TABLE 5
T cell number per cluster in brain parenchyma and meninges

| | | Total | Cell | |
|---|---|---|---|---|
| | | cell | num- | Per- |
| | | num- | ber/ | centage |
| Cluster | Region | ber | Cluster | (%) |
| 0 CD8⁺Xcl1⁺ | Parenchyma | 18565 | 3594 | 19.4 |
| 0 CD8⁺Xcl1⁺ | meninges | 5040 | 457 | 9.1 |
| 1 CD8⁺Tox⁺Pdcd1⁺ | Parenchyma | 18565 | 2607 | 14.0 |
| 1 CD8⁺Tox⁺Pdcd1⁺ | meninges | 5040 | 537 | 10.7 |
| 2 CD8⁺Ly6c⁺ | Parenchyma | 18565 | 2422 | 13.0 |
| 2 CD8⁺Ly6c⁺ | meninges | 5040 | 548 | 10.9 |
| 3 CD8⁺Isg15⁺ | Parenchyma | 18565 | 1635 | 8.8 |
| 3 CD8⁺Isg15⁺ | meninges | 5040 | 303 | 6.0 |
| 4 CD4⁺Folr4⁺Slamf6⁺ | Parenchyma | 18565 | 1096 | 5.9 |
| 4 CD4⁺Folr4⁺Slamf6⁺ | meninges | 5040 | 724 | 14.4 |
| 5 ll17⁺ | Parenchyma | 18565 | 1283 | 6.9 |
| 5 ll17⁺ | meninges | 5040 | 302 | 6.0 |
| 6 ll4⁺ | Parenchyma | 18565 | 1112 | 6.0 |
| 6 ll4⁺ | meninges | 5040 | 274 | 5.4 |
| 7 Cd4⁺Cd40lg⁺ | Parenchyma | 18565 | 923 | 5.0 |
| 7 Cd4⁺Cd40lg⁺ | meninges | 5040 | 341 | 6.8 |
| 8 Cd8⁺Egr1⁺ | Parenchyma | 18565 | 992 | 5.3 |
| 8 Cd8⁺Egr1⁺ | meninges | 5040 | 13 | 0.3 |
| 9 Cd8⁺Cd7⁺Gzmb⁺ | Parenchyma | 18565 | 1003 | 5.4 |
| 9 Cd8⁺Cd7⁺Gzmb⁺ | meninges | 5040 | 233 | 4.6 |
| 10 Cd8⁺Cd11c⁺Klre1⁺ | Parenchyma | 18565 | 820 | 4.4 |
| 10 Cd8⁺Cd11c⁺Klre1⁺ | meninges | 5040 | 154 | 3.1 |
| 11 Cd8⁺Ccr7⁺Sell⁺ | Parenchyma | 18565 | 255 | 1.4 |
| 11 Cd8⁺Ccr7⁺Sell⁺ | meninges | 5040 | 464 | 9.2 |
| 12 Cd8⁺Cx3cr1⁺Gzma⁺ | Parenchyma | 18565 | 220 | 1.2 |
| 12 Cd8⁺Cx3cr1⁺Gzma⁺ | meninges | 5040 | 353 | 7.0 |
| 13 Cd4⁺Foxp3⁺ | Parenchyma | 18565 | 286 | 1.5 |
| 13 Cd4⁺Foxp3⁺ | meninges | 5040 | 287 | 5.7 |
| 14 Cd4⁺Bcl3⁺Relb⁺ | Parenchyma | 18565 | 317 | 1.7 |
| 14 Cd4⁺Bcl3⁺Relb⁺ | meninges | 5040 | 50 | 1.0 |

TABLE 6
CD4⁺ T cell number per cluster in brain parenchyma

| | | Total cell | Cell |
|---|---|---|---|
| Cluster | Replica | number | number/Cluster |
| 0 Egr1⁺ | Rep1 | 2319 | 319 |
| 0 Egr1⁺ | Rep1 | 2319 | 229 |
| 1 | Rep1 | 2319 | 171 |
| Nkg7⁺Ccl5⁺ | | | |
| 1 | Rep2 | 2319 | 95 |
| Nkg7⁺Ccl5⁺ | | | |
| 2 Rpl⁺ | Rep1 | 2319 | 409 |
| 2 Rpl⁺ | Rep2 | 2319 | 284 |
| 3 | Rep1 | 2319 | 185 |
| Slamf6⁺Klf2⁺ | | | |
| 3 | Rep2 | 2319 | 174 |
| Slamf6⁺Klf2⁺ | | | |
| 4 | Rep1 | 2319 | 111 |
| Cxcr6⁺Ccr8⁺ | | | |
| 4 | Rep2 | 2319 | 86 |
| Cxcr6⁺Ccr8⁺ | | | |
| 5 Foxp3⁺ | Rep1 | 2319 | 103 |
| 5 Foxp3⁺ | Rep2 | 2319 | 153 |

TABLE 7
CD8⁺ T cell number per cluster in brain parenchyma

| | | | Cell |
|---|---|---|---|
| | | Total cell | number/ |
| Cluster | Replica | number | Cluster |
| 0 Rpl⁺ | Rep1 | 9447 | 1526 |
| 0 Rpl⁺ | Rep2 | 9447 | 1444 |
| 1 Tox⁺Pdcd1⁺ | Rep1 | 9447 | 932 |
| 1 Tox⁺Pdcd1⁺ | Rep2 | 9447 | 829 |
| 2 Ly6c⁺ | Rep1 | 9447 | 510 |
| 2 Ly6c⁺ | Rep2 | 9447 | 457 |
| 3 Isg15⁺ | Rep1 | 9447 | 898 |
| 3 Isg15⁺ | Rep2 | 9447 | 750 |
| 4 | Rep1 | 9447 | 342 |
| Hobit⁺Cd40lg⁺ | | | |
| 4 | Rep2 | 9447 | 167 |
| Hobit⁺Cd40lg⁺ | | | |
| 5 Egr1⁺ | Rep1 | 9447 | 403 |
| 5 Egr1⁺ | Rep2 | 9447 | 304 |
| 6 | Rep1 | 9447 | 221 |
| Cd11c⁺Klre1⁺ | | | |
| 6 | Rep2 | 9447 | 100 |
| Cd11c⁺Klre1⁺ | | | |
| 7 Ccr7⁺Sell⁺ | Rep1 | 9447 | 135 |
| 7 Ccr7⁺Sell⁺ | Rep2 | 9447 | 77 |
| 8 Bcl3⁺Relb⁺ | Rep1 | 9447 | 122 |
| 8 Bcl3⁺Relb⁺ | Rep2 | 9447 | 114 |
| 9 | Rep1 | 9447 | 88 |
| Cx3cr1⁺Gzma⁺ | | | |
| 9 Cx3cr1⁺Gzma⁺ | Rep2 | 9447 | 28 |

### Example 5: Interaction of Activated Microglia and T Cells

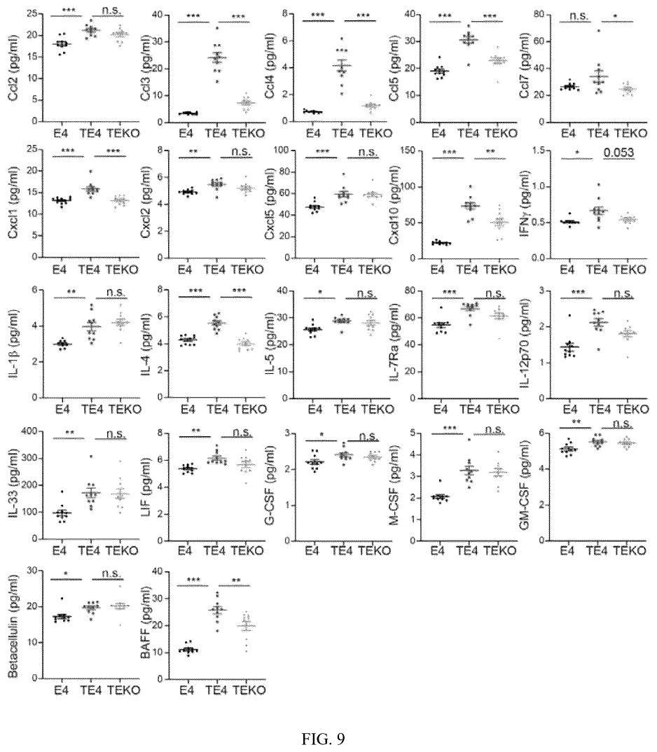

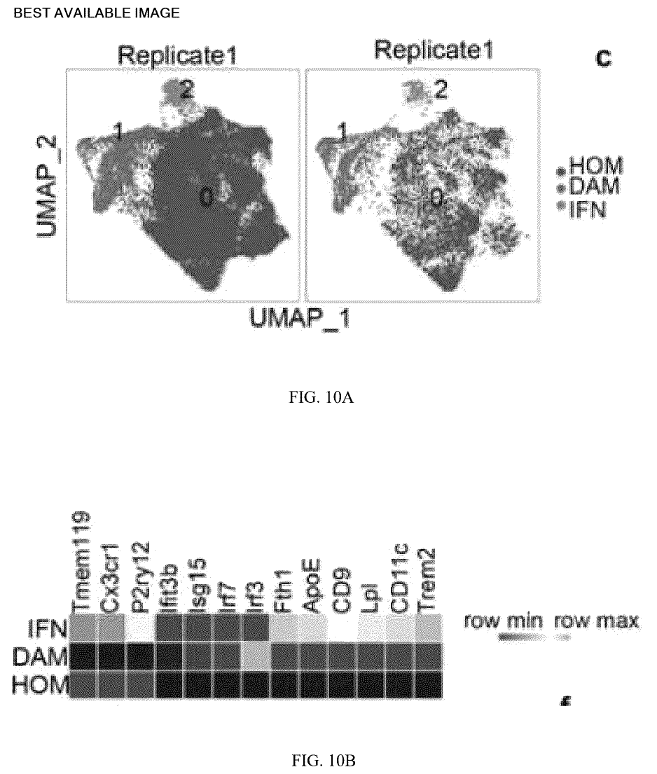

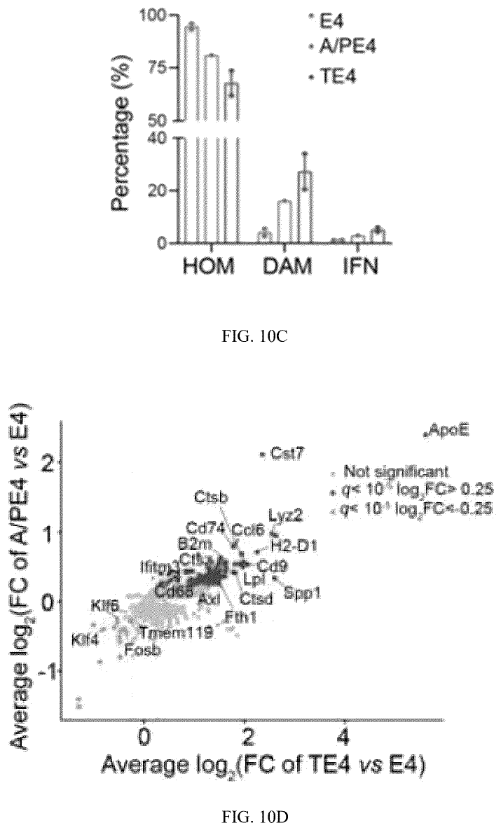

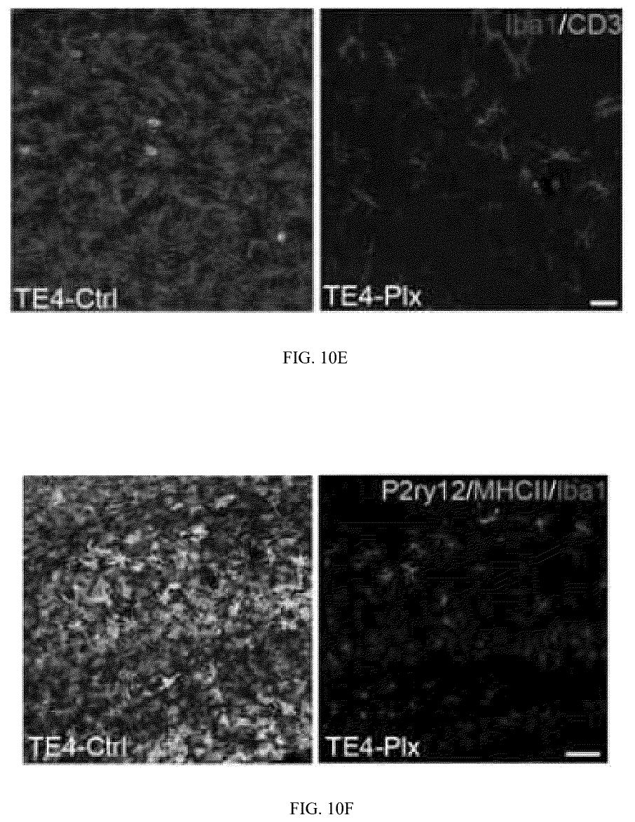

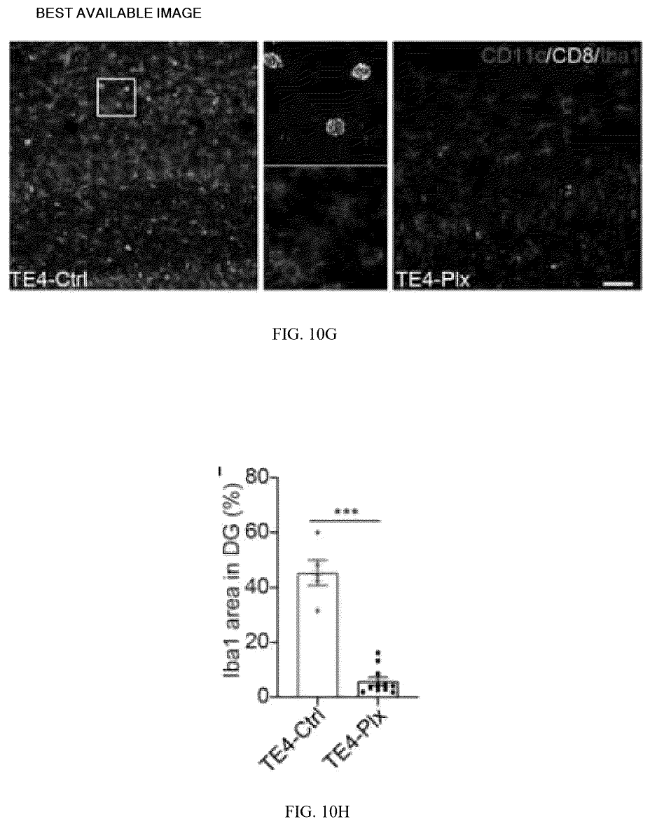

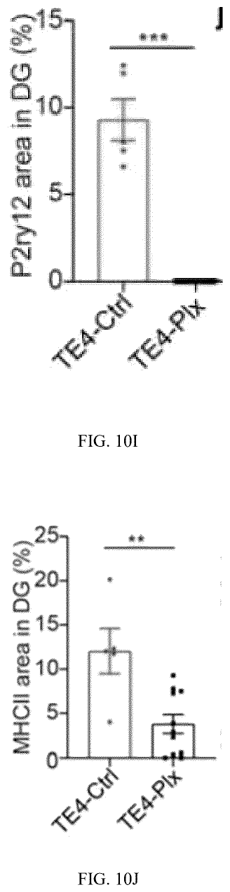

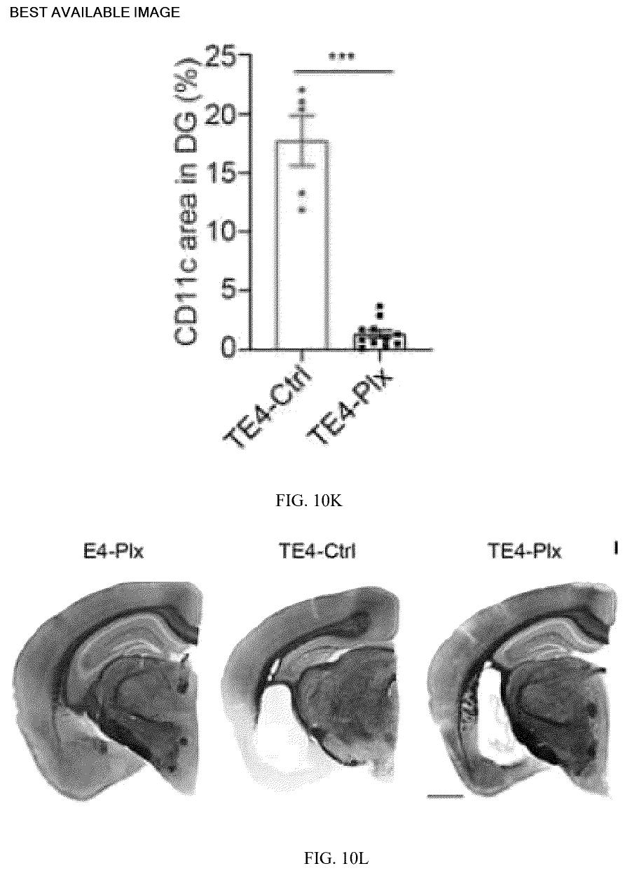

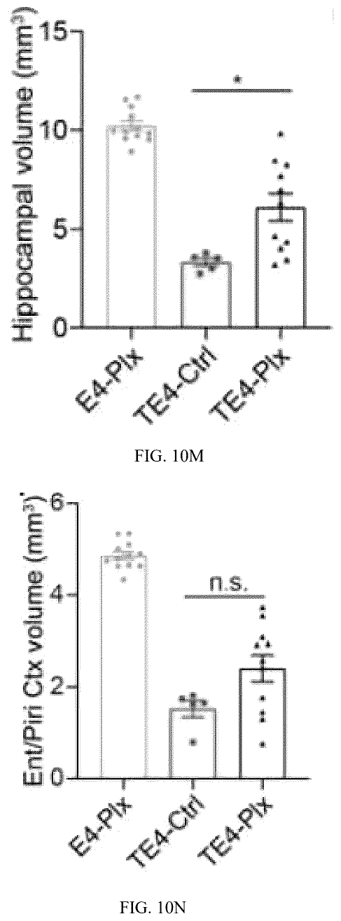

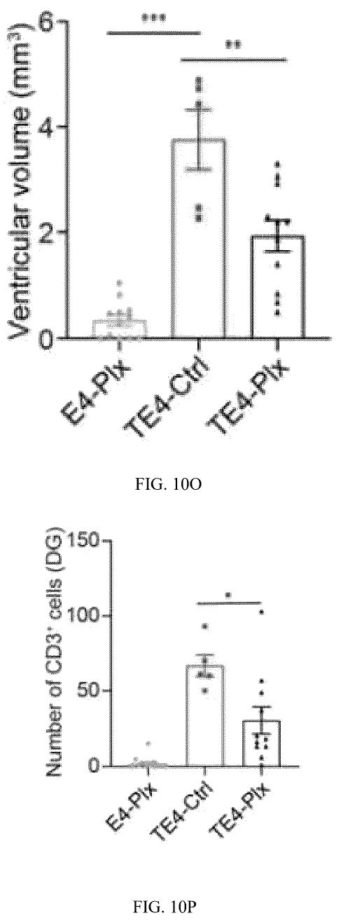

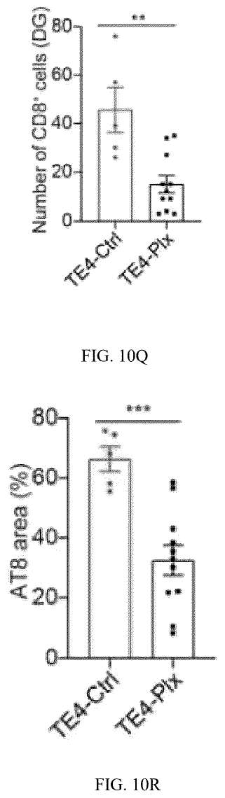

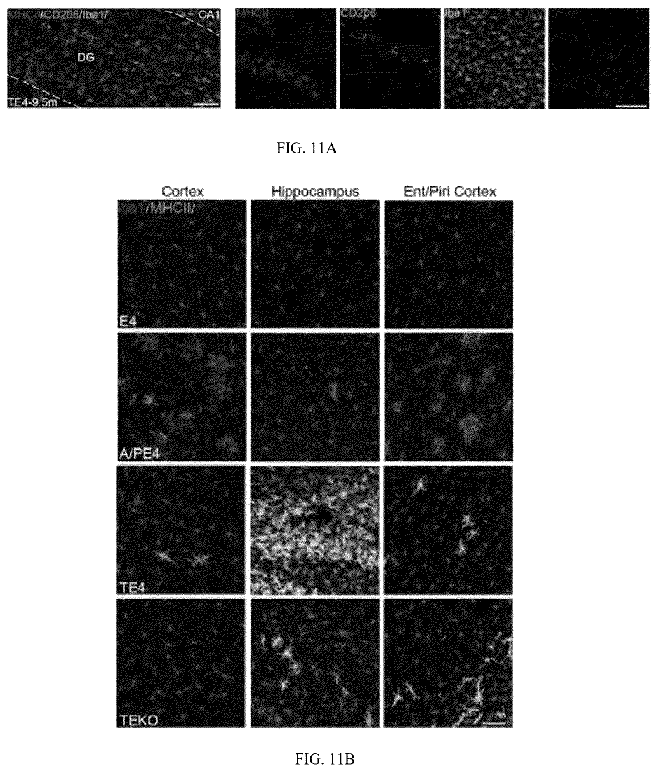

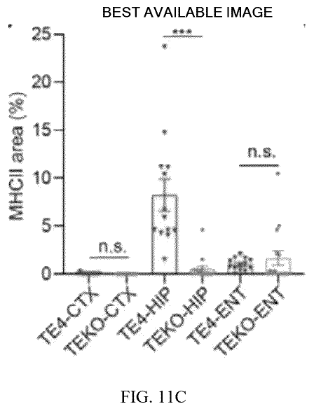

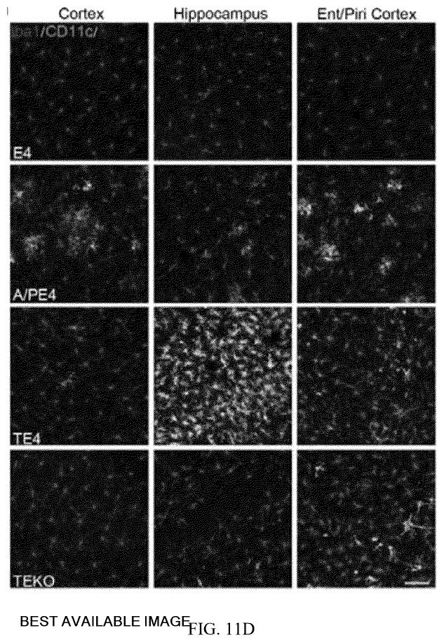

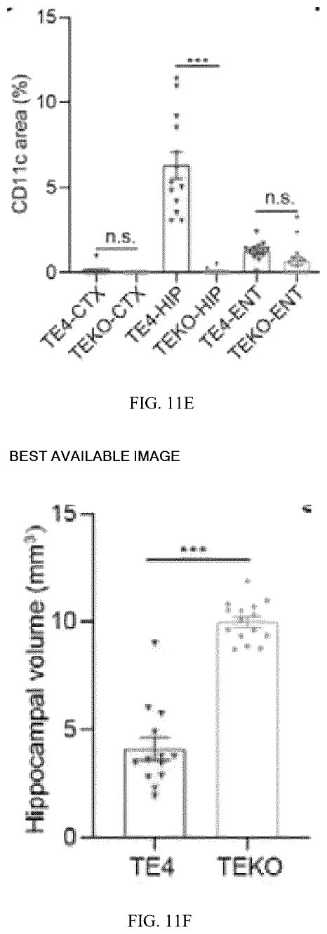

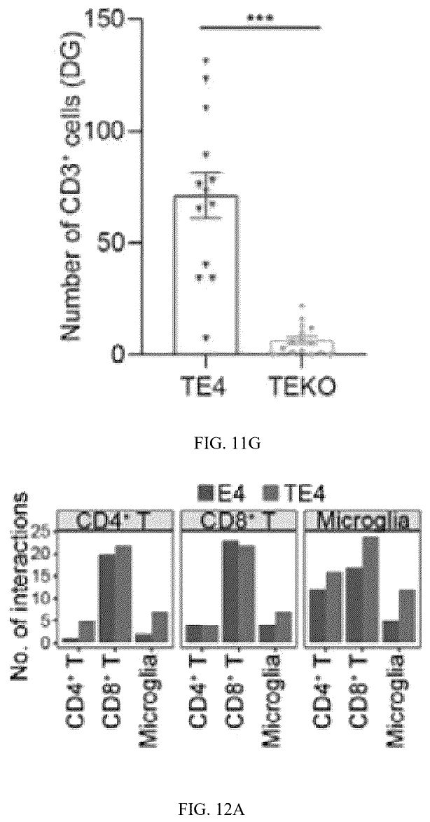

Next, the unique but complex immune hubs were explored in the parenchyma of tauopathy brains, which lead to T cell homing and activation. Notably, CCL3, CCL4 and CXCL10, chemokines previously reported to be associated with T cell chemotaxis and brain infiltration were increased in the brain lysates of TE4 mice compared to E4 and TEKO mice (FIG. 9). Microglia are the first responders to neuroinflammation or damage and they rapidly adapt their phenotypes and functions in response to the dynamic brain milieu. Typical functions of microglia such as phagocytosis and cytokine production have been well characterized in models of neurodegeneration including AD35,36; however, whether they exert their effects via their interactions with T cells is largely unknown. Microglia were sub-grouped (cell type 0, FIG. 1F) from the CD45^{Total} population of E4, A/PE4 and TE4 mice. Three subgroups were obtained with distinguishing markers associated with homeostatic microglia (HOM), disease associated microglia (DAM) and interferon-activated microglia (IFN) (FIG. 10A, FIG. 10B and Table 8). Notably, DAM and IFN subgroups were strongly elevated in TE4 mice, while the HOM subgroup decreased (FIG. 10C). Genes related to antigen presentation, complement response and cytokines, metabolism and oxidative stress, together with lysosomal enzymes were found upregulated in TE4 mice to a greater extent compared to A/PE4 mice, which were greater than the control mice (FIG. 10D). Classically, MHCI and MHCII enable antigen presentation to CD8⁺ T cells and CD4⁺ T cells, respectively. MHCI is expressed by all nucleated cells, while MHCII is only expressed by antigen presenting cells (APCs), such as DCs, macrophages, B cells and microglia. By co-staining with the perivascular macrophage marker, MRC1 mannose receptor C (CD206), in addition to MHCII, Iba1 and GFAP, MHCII was found to be primarily present in Iba1⁺ microglia in the brain parenchyma in neurodegeneration regions (FIG. 11A). Indeed, in line with the increase in parenchymal T cells, MHCII⁺ microglia were found significantly elevated in brain regions with tau pathology in TE4 mice (FIG. 11B, FIG. 11C). DAM and its sub-types have been well characterized in amyloid models. In TE4 mice, Integrin, αx (CD11c) positive microglia, a representative marker for myeloid cell 2 (Trem2)-dependent type 2 DAM, were found physically co-localized with CD8⁺ T cells. CD11c was also strongly increased in TE4 hippocampus as compared to the A/PE4 and E4 control mice (FIG. 11D, FIG. 11E). These results highlight a tight correlation with MHCII⁺ microglia, CD11c⁺ microglia, T cells and neurodegeneration. Sparse MHCII⁺ microglia and CD11c⁺ microglia were also found co-localized with parenchymal plaques in A/PE4 mice (FIG. 11B, FIG. 11D). ApoE deletion rescued brain atrophy in P301S Tau: ApoE KO (TEKO) mice and MHCII⁺ and CD11c⁺ microglia as well as T cells were significantly decreased (FIG. 11B-G). The higher inflammatory reactivity associated with Tau-mediated neurodegeneration and ApoE were also confirmed by assessment of inflammatory cytokines in brain tissue from TE4 and TEKO mice (FIG. 9). In summary, the data demonstrated that parenchymal microglia, in the presence of tauopathy, shift their transcriptomic and phenotypical states from homeostatic to disease-associated, CD11c⁺, MHCII⁺ and IFN-activated states, with accompanying increase in inflammatory chemokine and cytokines.

TABLE 8
Microglia number per cluster in brain parenchyma

| | | | Total | Cell | Per- |
|---|---|---|---|---|---|
| | Geno- | Re- | cell | number/ | centage |
| Cluster | type | plica | number | Cluster | (%) |
| 0 Homeostatic | E4 | Rep1 | 16951 | 16265 | 96.0 |
| microglia | | | | | |
| 0 Homeostatic | E4 | Rep2 | 2901 | 2702 | 93.1 |
| microglia | | | | | |
| 0 Homeostatic | A/PE4 | Rep1 | 7502 | 6072 | 80.9 |
| microglia | | | | | |
| 0 Homeostatic | TE4 | Rep1 | 6210 | 4566 | 73.5 |
| microglia | | | | | |
| 0 Homeostatic | TE4 | Rep2 | 3341 | 2061 | 61.7 |
| microglia | | | | | |
| 1 APOE⁺ microglia | E4 | Rep1 | 16951 | 455 | 2.7 |
| 1 APOE⁺ microglia | E4 | Rep2 | 2901 | 162 | 5.6 |
| 1 APOE⁺ microglia | A/PE4 | Rep1 | 7502 | 1206 | 16.1 |
| 1 APOE⁺ microglia | TE4 | Rep1 | 6210 | 1267 | 20.4 |
| 1 APOE⁺ microglia | TE4 | Rep2 | 3341 | 1137 | 34.0 |
| 2 IFN-activated | E4 | Rep1 | 16951 | 231 | 1.4 |
| microglia | | | | | |
| 2 IFN-activated | E4 | Rep2 | 2901 | 37 | 1.3 |
| microglia | | | | | |
| 2 IFN-activated | A/PE4 | Rep1 | 7502 | 224 | 3.0 |
| microglia | | | | | |
| 2 IFN-activated | TE4 | Rep1 | 6210 | 377 | 6.1 |
| microglia | | | | | |
| 2 IFN-activated | TE4 | Rep2 | 3341 | 143 | 4.3 |
| microglia | | | | | |

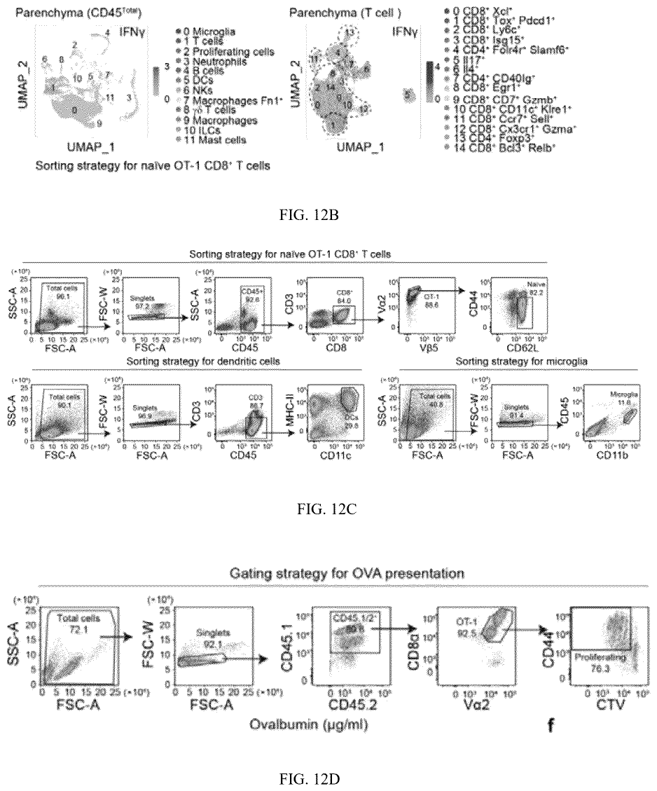

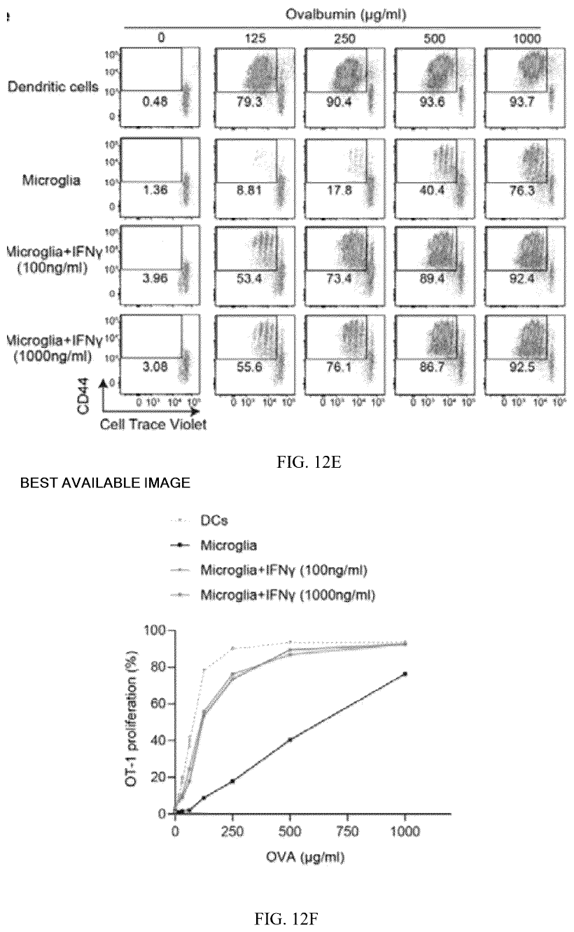

IFN-γ, a cytokine upregulated in TE4 mice, is a proinflammatory cytokine produced by NK, NKT, and T cells that can prime microglia for inflammatory responses to injury as well as promote cytotoxic CD8⁺ T cell function. Previous studies identified IFN-γ related transcriptomic signatures in tauopathy and neurodegenerative disease models, although the cell-type expression and functional result of IFN-γ on pathology was not described. Ligand-receptor analysis revealed active interactions within T cells and microglia (FIG. 12A). IFN-γ receptor has been previously shown to be expressed in both neurons and microglia in the brain. However, it was found that in the brain of TE4 mice, IFN-γ transcripts were enriched in T cells, especially CD8⁺ T cells (FIG. 12B). Given that IFN-γ can augment antigen presenting and inflammatory functions of myeloid cells, the role of IFN-γ was further investigated in the immune response in tauopathy. To determine whether microglia can present antigen to T cells in vitro, microglia acutely isolated from adult mouse brain was co-cultured with OT-1 T cells, with soluble ovalbumin (OVA) as antigen, and found that microglia were capable of weakly stimulating OT-1 T cell proliferation compared to DCs (FIG. 12C-F). However, upon IFN-γ stimulation, OT-1 T cell proliferation was strongly enhanced in the presence of microglia with OVA (FIG. 12E, FIG. 12F), nearly to the level observed with DCs, suggesting that microglia in vitro can serve as antigen presenting cells to T cells and that IFN-γ can augment this response. In summary, these data suggest the possibility that there are active interactions between microglia and infiltrated T cells.

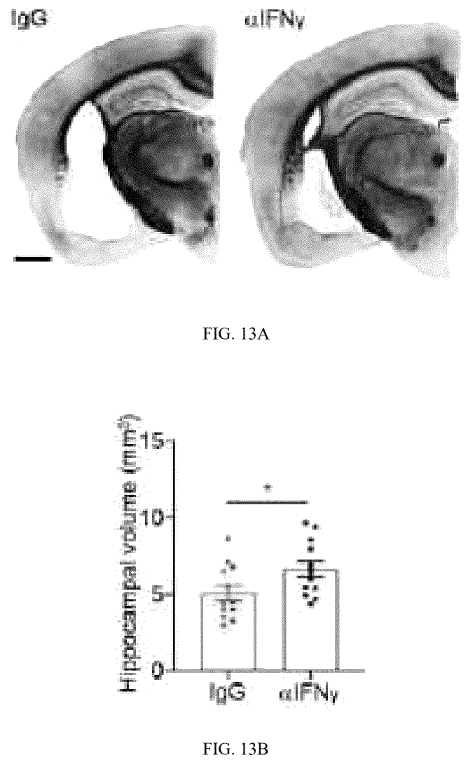

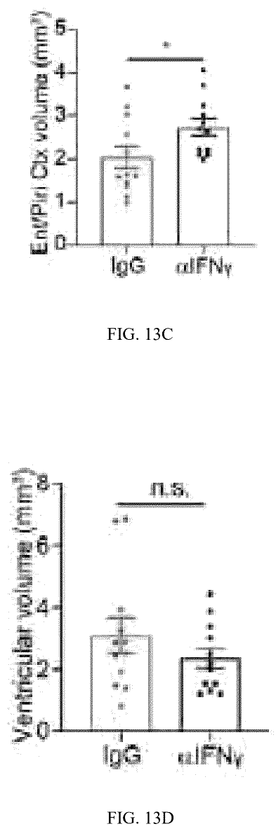

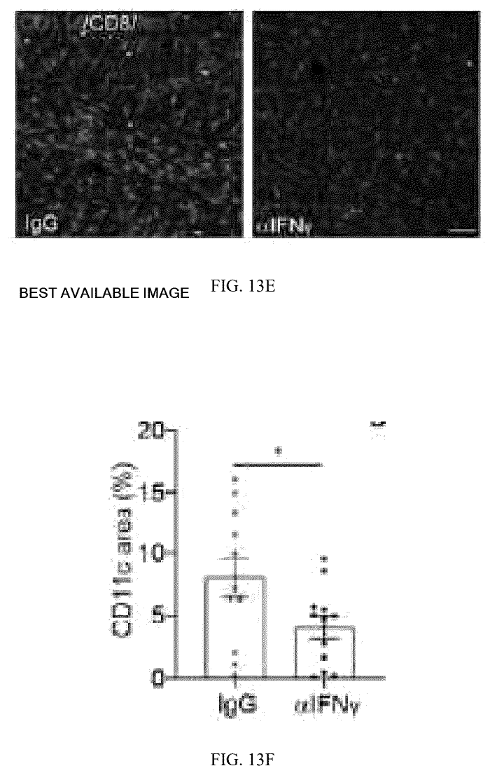

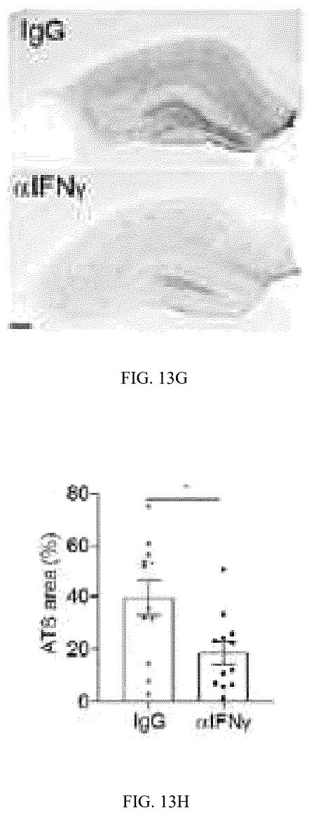

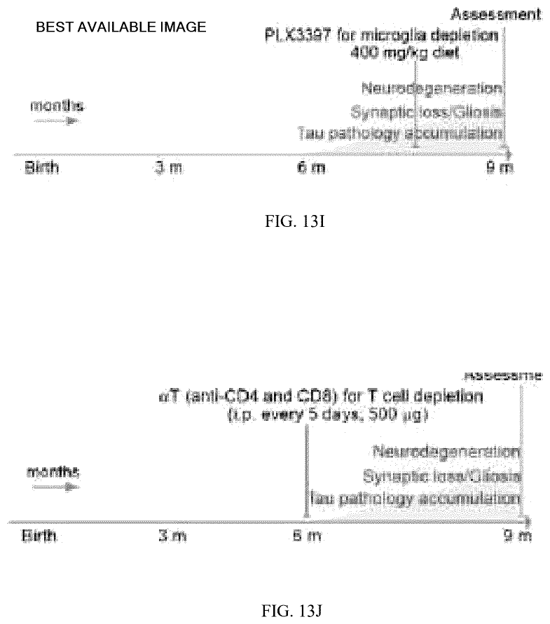

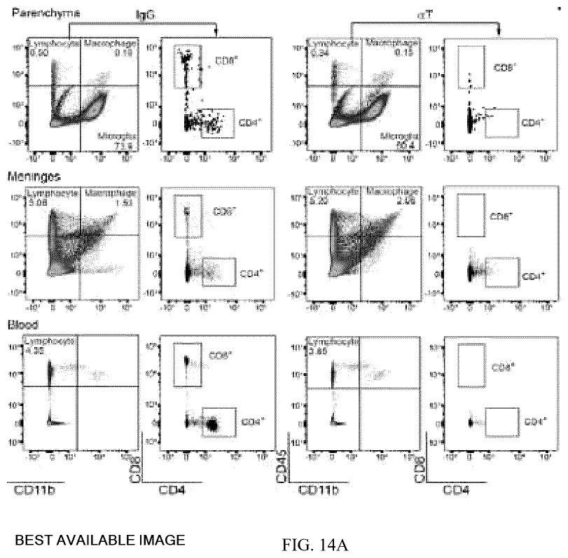

To determine the role of endogenous IFN-γ in the P301S mice in vivo and to study the interplay between activated microglia and T cells, IFN-γ signaling was blocked using peripheral administration i.p. every five days with a neutralizing antibody in TE3 mice from 7.5 to 9.5 months of age, right before T cell infiltration into the brain parenchyma. Anti-IFN-γ (αIFN-γ) treatment resulted in attenuated brain atrophy as compared to the IgG treatment control (FIG. 13A-D). CD11c⁺ microglia were also significantly reduced in αIFN-γ treated mice (FIG. 13E, FIG. 13F) and there was a significant reduction in p-tau staining in αIFN-γ treated mice (FIG. 13G, FIG. 13H). Taken together, these results suggest that IFN-γ secreted by CD8⁺ T cells in the CNS can augment tau pathology and neurodegeneration, at least in part through promoting inflammatory microglial signaling and antigen presentation functions.

To further delineate the interrelationship between the activated microglia and infiltrated T cells, PLX3397, a selective CSF1R/c-kit/FLT3 inhibitor that has been shown to effectively remove microglia, was administered in TE4 and E4 control mice from 8.5 months to 9.5 months of age (FIG. 13I). PLX3397 treatment resulted in strong microglial depletion (FIGS. 10E-K). PLX3397 treatment also decreased hippocampal atrophy and ameliorated the increased of ventricular volume in TE4 mice (FIGS. 10L-O). Notably, CD3⁺ and CD8⁺ T cells as well as tau pathology were reduced upon microglia depletion (FIGS. 10P-R), suggesting a pivotal role of microglia, especially activated microglia, in setting of the tauopathy-specific immune hubs by recruiting and activation T cells into the brain parenchyma and a detrimental role of this re-structured immune hub in facilitating disease progression.

### Example 6: T Cell Depletion Protects Against Tauopathy and Degeneration

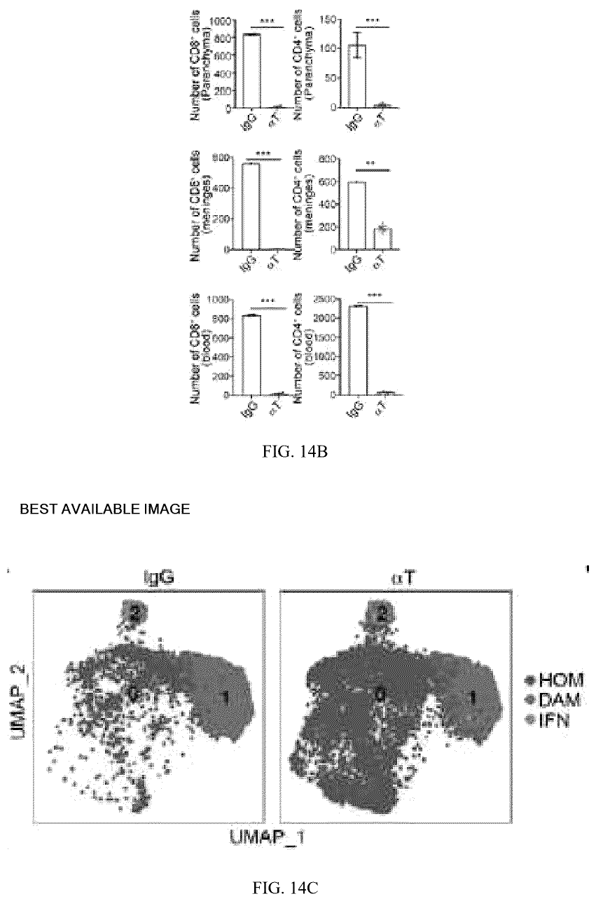

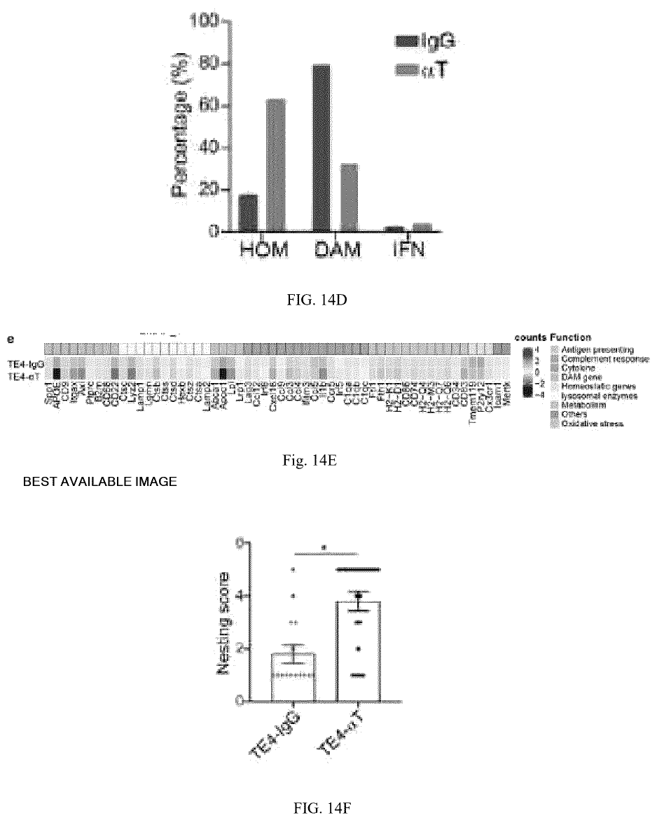

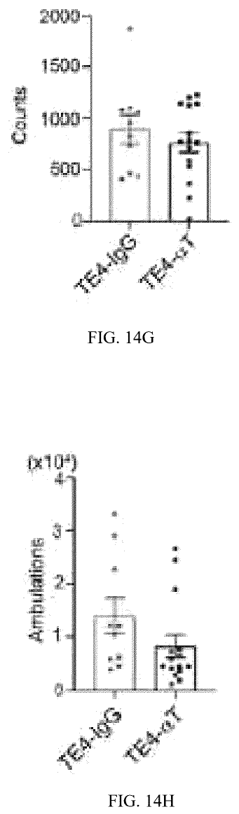

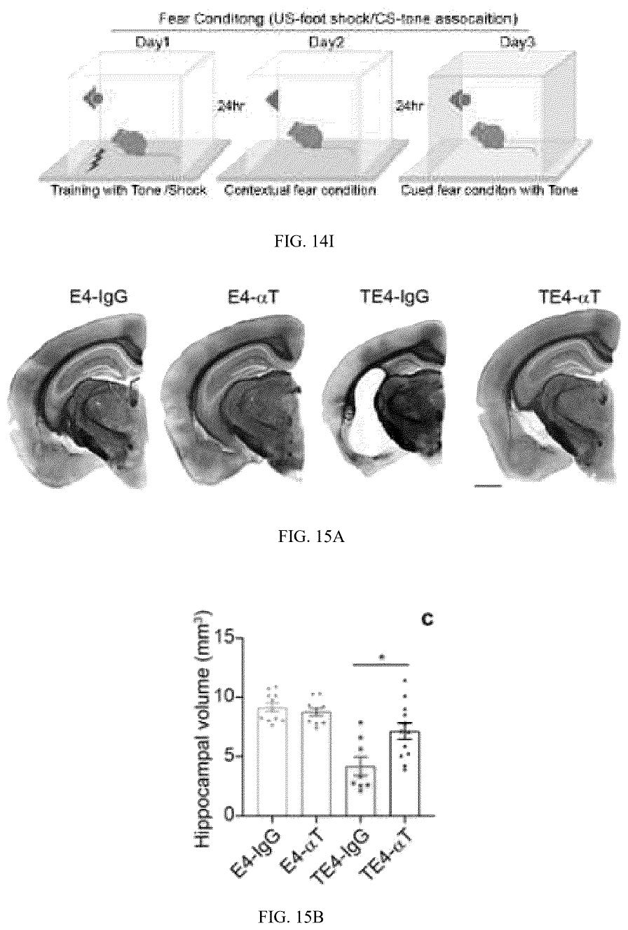

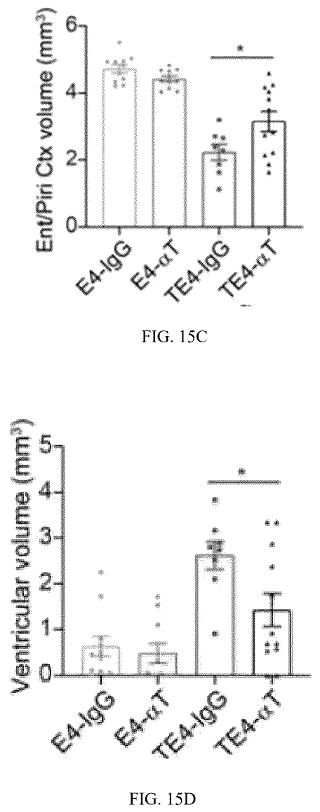

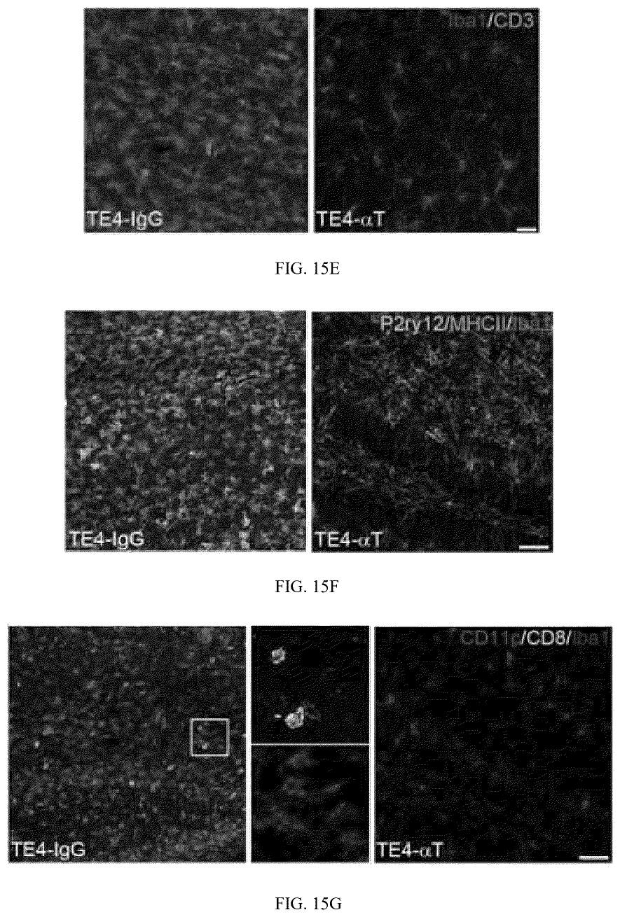

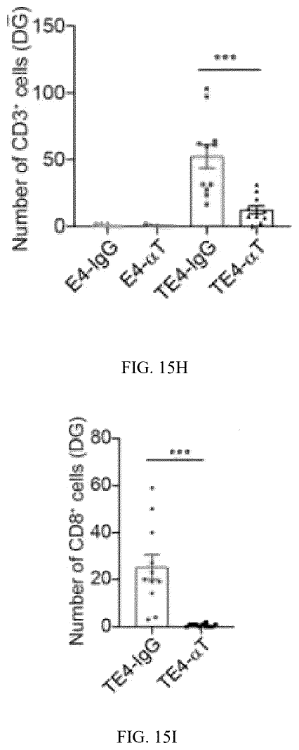

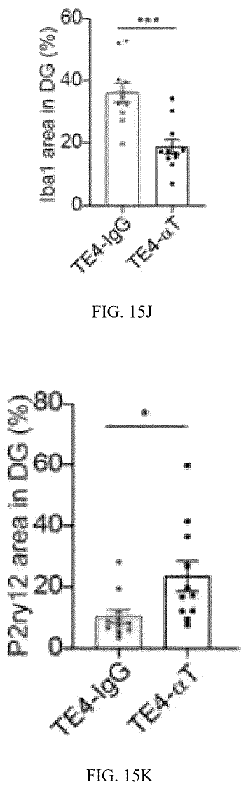

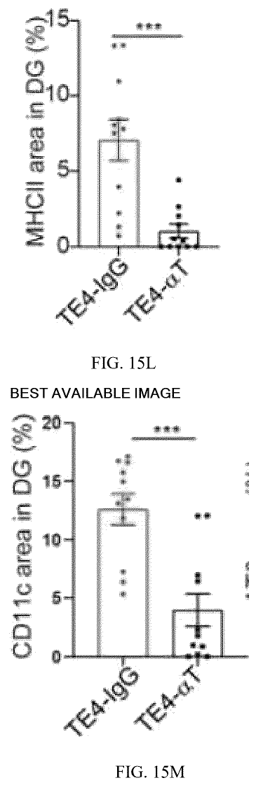

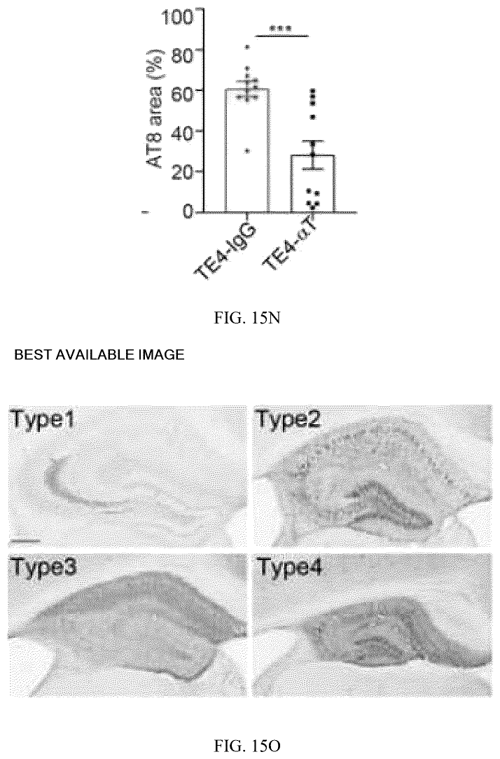

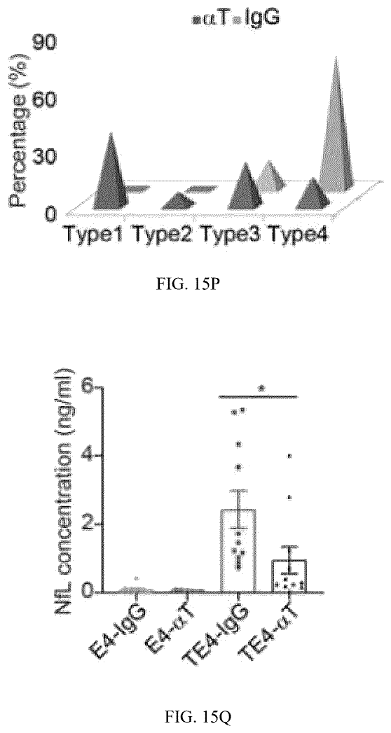

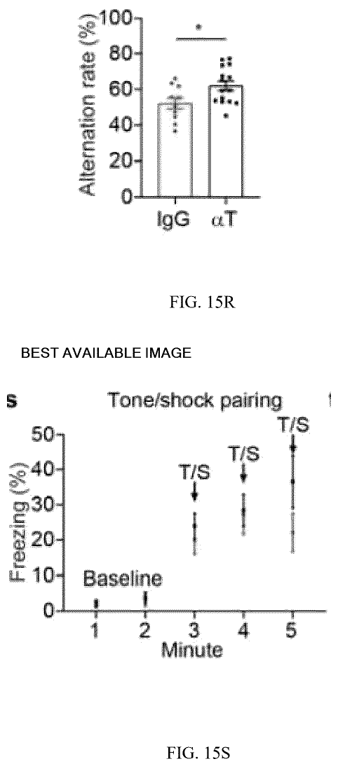

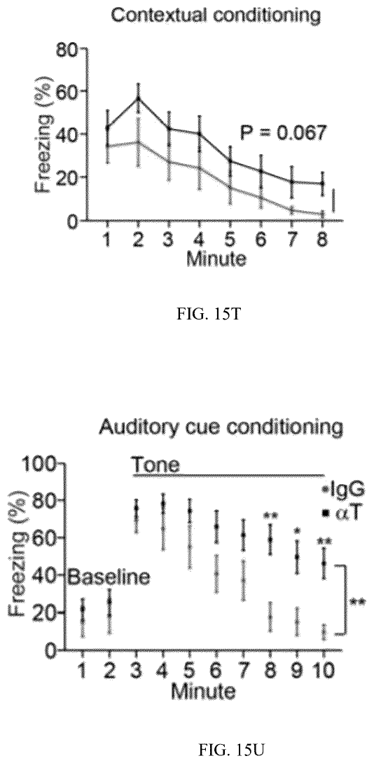

To directly investigate whether infiltration of T cells leads to neurodegeneration, T cells were depleted by peripheral administration of neutralizing antibodies in TE4 mice as well as their age-matched non-tau transgenic littermates from 6 months to 9.5 months of age, a critical time window when neurodegeneration develops (FIG. 13J). A single dose acute i.p. treatment with anti-CD4 (αCD4) and anti-CD8 (αCD8) antibodies led to strong CD4 and CD8 T cells depletion in brain parenchyma, meninges and peripheral blood, confirming the antibody depletion efficiency (FIG. 14A, FIG. 14B). In TE4 mice with aT treatment (i.p. every 5 days) from 6 months to 9.5 months, brain atrophy was strongly ameliorated compared to IgG treated control (FIG. 15A-D). T cells were almost completely eliminated in the brain parenchyma in TE4 mice after 3.5 months of aT antibody treatment (FIG. 15E, FIG. 15G, FIG. 15H, FIG. 15I). Interestingly, T cell depletion also reduced overall microglial staining (FIG. 15E, FIG. 15F, FIG. 15G, FIG. 15J), suggesting that T cells in the brain of TE4 mice can augment microgliosis. To assess the activation status of microglia with and without T cells depletion, immunohistochemical analyses was performed in the parenchyma from TE4-IgG and TE4-αT treated mice using antibodies to P2ryl2, MHCII and CD11c (FIG. 15F, FIG. 15G). A significant elevation of P2ryl2⁺ microglia and reduced MHCII⁺ and CD11c⁺ microglia was observed (FIG. 15K-M) in the aT antibody treated mice, suggesting microglia shift from activated towards a more homeostatic state after T cell depletion. Single cell RNA seq analysis of microglia from aT antibody vs. the IgG control treated mice also revealed strong suppression of different aspects of the disease related microglia signature and an increase in the homeostatic signature (FIG. 14C-E). To assess tau pathology following T cell depletion, p-Tau immunoreactivity was analyzed in hippocampus and a significant reduction in p-Tau was observed in aT treated mice (FIG. 15N). Four major p-tau staining patterns, designated as type1-4, strongly correlated with the level of brain atrophy, with type 1 associated with most preserved brain tissue and type 4 associated with the greatest atrophy. Depletion of T cells resulted in a significant shift of p-Tau staining pattern toward the earliest disease stage (FIG. 15O, FIG. 15P). Plasma protein levels of neurofilament light chain (NfL), a marker of neuroaxonal damage and neurodegeneration was also assessed. NfL concentration in T cells depleted mice was significantly reduced (FIG. 15Q). Behavioral performance assessment reveal that after depletion of T cells, nest-building behavior in TE4 mice was significantly improved (FIG. 14F). An additional cohort of TE4 mice that were treated with aT antibody vs. the IgG control from 6 months to 8.5 months of age were assessed. Assessment of behavioral performance revealed that depletion of T cells resulted in significant improvement in 2 additional behaviors. Y maze alternation, assessing short-term memory and exploratory behavior, as well as freezing in response to auditory cue, assessing amygdala-dependent memory, were significantly improved (FIGS. 15R-U and FIGS. 14G-I). Freezing behavior in response to a contextual cue showed a trend toward increased hippocampal dependent memory after depletion of T cells (FIG. 15T). Both groups showed similar baseline levels of general exploratory behavior, and locomotor activity levels (FIGS. 14G-H) and response to tone/shock pairing in the fear conditioning test (FIG. 15S). Together, these data demonstrate that T cell depletion decreases functional decline.

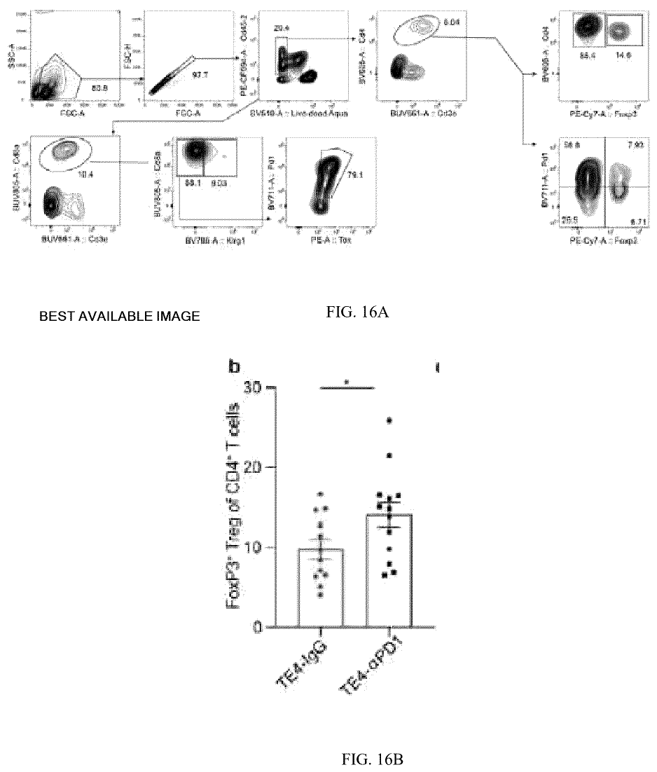

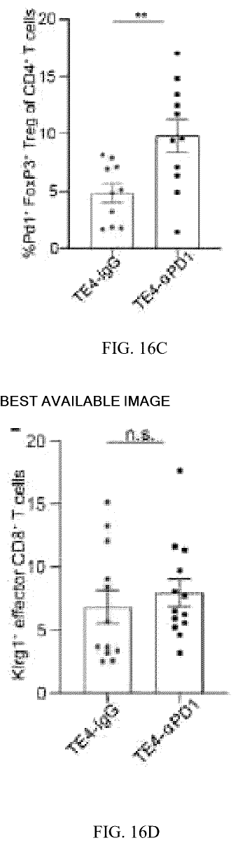

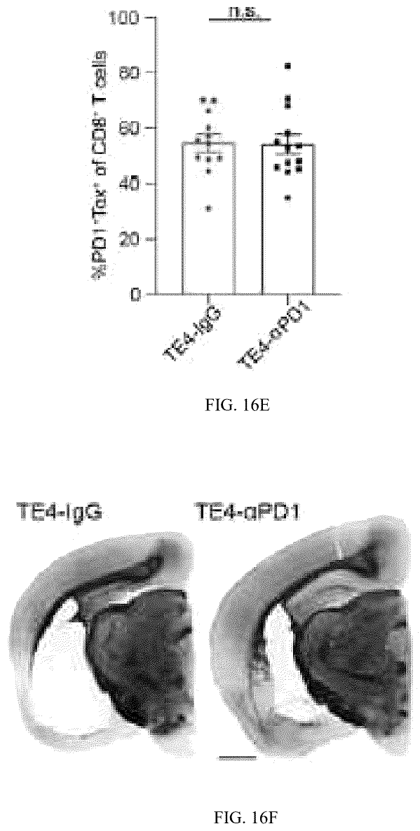

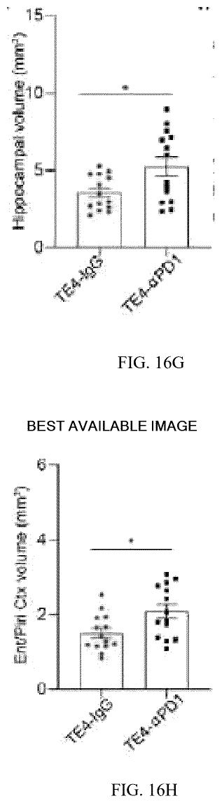

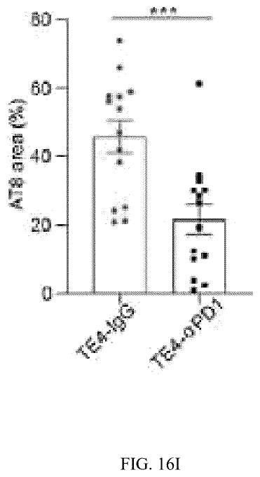

To investigate whether a treatment that targets PD-1/PD-L1 blockade could be effective in tauopathy, anti-PD-1 (αPD-1) treatment was administered in TE4 mice from 8 months to 9.5 months of age, a time window that brain atrophy dramatically develops. It was found that, a one-week acute treatment increased the percentage of Foxp3⁺CD4⁺ Tregs and Pd1⁺Foxp3⁺CD4⁺ Tregs, with no obvious changes on Klrg1⁺ effector CD8⁺ T cells and total Pd1⁺ Tox⁺CD8⁺ T cells in the brain (FIGS. 16A-E). These results suggested that PD1-antibody treatment at this age would increase immunosuppressive CD4 Tregs. Consistently, chronic treatment beginning at 8 months significantly decreased tau-mediated neurodegeneration and p-tau staining (FIGS. 16F-I), further supporting a role for T cells in tau-mediated neurodegeneration.

### Example: P301S Tau Transgenic Mice are Protected Against Neurodegeneration when Crossed to Rag2KO Mice

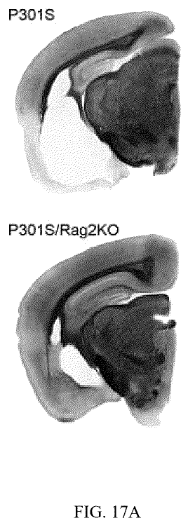

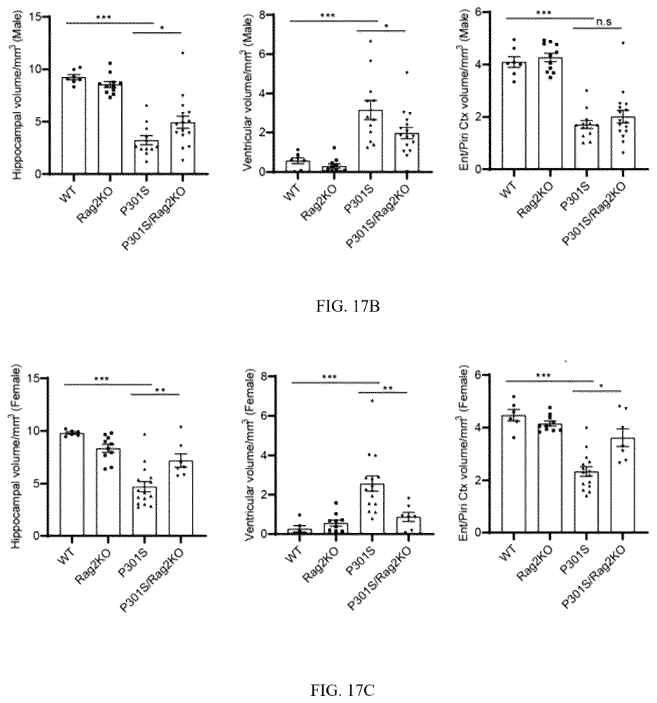

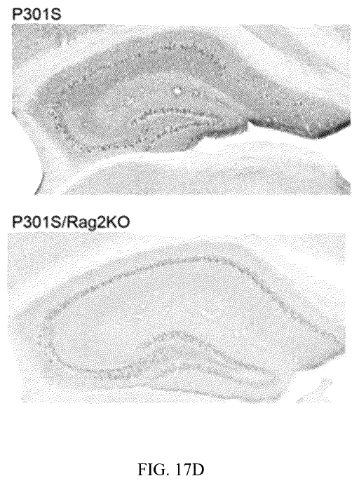

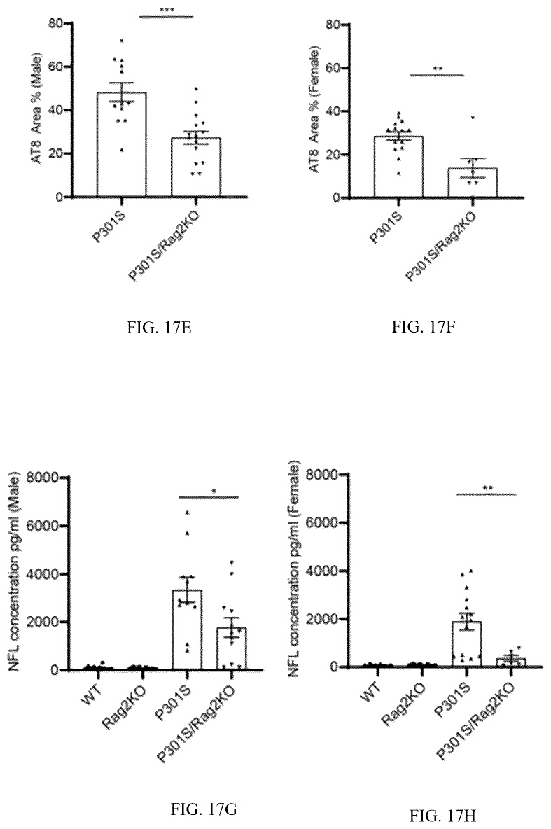

P301S mice on a C57BI/6J background were bred for 2 generations to Rag2KO mice (also on a C37BI/6J background). Rag2KO mice lack both T cells and B cells. The further supports the findings that T cells are critical mediators of neurodegeneration due to tau pathology. P301S mice and P301S/Rag2KO mice (male and female) were aged to 285 days of age (9.5 months of age). Mice were then sacrificed and perfused transcardially with phosphate buffered saline for 3 minutes. Brains were then removed, and ½ the brain was fixed in 4% paraformaldehyde for 24 hours at 40° C. followed by 30% sucrose for 24 hours at 40° C. Samples were frozen and cut on a freezing sliding microtome and processed for Sudan black staining as well as staining for phosphorylated tau with antibody AT8 as described in previous examples. At time of sacrifice, plasma was collected from each mouse and plasma neurofilament light chain (NFL) was measured as described in previous examples. Results for immunofluorescent staining and quantification, volumetric measurements, and NFL measurements are shown in FIGS. 17A-17H.

### Summary of Examples

In the disclosed experiments, comprehensive cellular and molecular immune response map in the brain and meninges during the development of amyloid or tau pathology and neurodegeneration was presented via single cell immune RNA-seq and scTCR-seq. It was found that an immunological hub involving activated microglia and T cells were overrepresented in brain regions with tauopathy and neuronal loss. Immune microenvironment in tauopathy by assessing a previously less examined adaptive immunological response was undertaken. A dynamic shift in T cells from activated to exhausted states with unique TCR clonal expansion was observed. The data presented direct evidence that breaking the neurodegeneration-featured immune hub between activated microglia and infiltrated T cells effectively decreased brain atrophy.

The immune system and the central nervous system (CNS) continuously surveil the environment and make on-demand adjustment to maintain their homeostasis. As an innate primary response, microglia appear to play an anti-(restrict plaque and inflammation expansion) or pro-inflammatory (response to neuronal damage and aggregated tau and lead to severe neurodegeneration) role in AD. Based on the above disclosed examples, it was found that CD11c and MHCII expression strongly increased in microglia specifically in brain atrophy regions. MHCI and MHCII complex genes were highly upregulated in activated microglia in tauopathy. Further, as adaptive responses, it was discovered that within both a mouse model of tauopathy and AD patient brain samples, T cells were not only present in the brain parenchyma but their enrichment also highly correlated with the severity of brain atrophy. Removal and modulation of T cells rescued the brain atrophy and highlighted that T cells played an important role in neurodegeneration.

The complex nature of the CNS necessitates its own specialized immunological adaptations to detect and respond to environmental changes. The disclosed examples showed a significantly different proportions of T cells in the meninges and brain parenchyma. These results indicated that CNS-border and CNS-resident T cells were functionally different in accordance with their immune niche. The local tauopathy-related microenvironment in the brain parenchyma was likely to be instructive for recruiting and guiding the transformation of T cells. Further, T cells actively were found to interact with the disease related microglia subgroups. Depletion of microglia largely abolished T cell infiltration and depletion of T cells also remarkably hindered microglia activation, strengthening immunological communication between the innate and adaptive family of immune cells. In combined single cell immune RNA-seq and scTCR-seq analyses, unique T cells clonal expansion were observed to be enriched in the parenchyma with tauopathy and neurodegeneration. The results further showed that T cell infiltration did not increase in the Tau mice lacking ApoE, indicating that immunomodulatory function of ApoE serve as an important mechanism linking both innate and adaptive immunity. It was also demostrated that P301S tau transgenic mice are protected against neurodegeneration, after crossing with Rag2KO mice, which lack mature B and T cells. Mapping the disease state-specific interlink between microglia and T cells, including their signaling communications, presented antigens, and pathophysiological responses will be a key nexus to set up unique therapeutic interventions to prevent or reverse brain atrophy in tauopathies.

1. A method of decreasing a condition selected from tau-mediated neurodegeneration, central nervous system (CNS) p-tau, brain atrophy, Nfl release from axons in the brain, and reactive microglia in the brain parenchyma in a subject in need thereof, the method comprising administering an agent to the subject that inhibits T-cell activation, decreases T-cell numbers, inhibits microglia activation, or decreases microglia number.

2. (canceled)

3. The method of claim 1, wherein the condition is brain atrophy and the subject is a tau positive subject.

4. (canceled)

5. The method of claim 1, wherein the condition is reactive microglia in the brain parenchyma and, the method comprises administering to the subject an agent that inhibits T-cell activation or decreases T-cell numbers in the brain parenchyma.

6. The method of claim 1, wherein the agent binds to CD80 or CD86.

7. The method of claim 6, wherein the agent inhibits or decreases T-cell activation.

8. The method of claim 6, wherein the agent is abatacept.

9. The method of claim 1, wherein the agent is an anti-VLA-4 antibody or an antigen-binding fragment thereof or a VLA-4 inhibitor.

10. The method of claim 9, wherein the anti-VLA-4 antibody is natalizumab.

11. (canceled)

12. The method of claim 9, wherein the VLA-4 inhibitor is GW559090.

13. The method of claim 1, wherein the agent inhibits the activity of, or decreases the amount of IFNgamma in the subject selected from an antibody or an antigen-binding fragment thereof and a small molecule.

14. (canceled)

15. (canceled)

16. The methods of claim 1, wherein the agent is a CSF1R/c-kit/FLT3 inhibitor.

17. The method of claim 16, wherein the agent is PLX3397.

18. The method of claim 1, wherein the agent reduces: (a) CD4+ cells; (b) CD8+ cells; or (c) both CD4+ and CD8+ cells.

19. (canceled)

20. (canceled)

21. The method of claim 1, wherein the agent is an antibody that specifically binds P2ryl2, MHCII or CD11c, or an antigen-binding fragment thereof.

22. The method of claim 1, wherein the agent is: (a) an immune checkpoint inhibitor; (b) an anti-PD-1 (αPD-1) antibody or antigen-binding fragment thereof; (c) fingolimod; or (d) a JAK inhibitor.

23. (canceled)

24. The method of claim 1, wherein the microglia are: (a) disease associated microglia; or (b) interferon-activated microglia.

25. (canceled)

26. A method of decreasing brain atrophy associated with tau pathology in a subject in need thereof, the method comprising administering an agent to the subject that disrupts the interaction between activated microglia and parenchyma infiltrated T cells in the brain of the subject.

27. The method of claim 26, wherein the subject: (a) has or is suspected of having Alzheimer's disease; or (b) has tau pathology but no clinical signs of Alzheimer's disease.

28. (canceled)

29. The method of claim 26, where the agent is: (a) fingolimod; or (b) a JAK inhibitor.

30. (canceled)
